SEQUENCE LISTING (38 sequences)

SEQ ID NO: 1 - protein, 194 aa, unidentified
  source 1..194: note = Humicola insolens
QLGAIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 2 - protein, 298 aa, unidentified
  source 1..298: note = Caldimonas brevitalea
MPPDCVLPRRLAAAALLASATLVPLSAAAQTNPYQRGPDPTTRDLEDSRGPFRYASTNVRSPSGYGAGTIYYPTDVSGSVGAVAVVPGYLARQSSIRWWGPRLASHGFVVITLDTRSTSDQPASRSAQQMAALRQVVALSETRSSPIYGKVDPNRLAVMGWSMGGGGTLISARDNPSLKAAVPFAPWHNTANFSGVQVPTLVIACENDTVAPISRHASSFYNSFSSSLAKAYLEINNGSHTCANTGNSNQALIGKYGVAWIKRFVDNDTRYSPFLCGAPHQADLRSSRLSEYRESCPY

SEQ ID NO: 3 - protein, 290 aa, unidentified
  source 1..290: note = Ideonella sakaiensis strain NBRC 110686 / TISTR 2288 / 201-F6
MNFPRASRLMQAAVLGGLMAVSAAATAQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 4 - protein, 426 aa, unidentified
  source 1..426: note = Burkholderiales bacterium
MAVGSMLLSMAAQAQVVVFEETFSTGLGKFTAAGSVVTSSGAARLDGCYGCTDGSITSTAISTVDFTGLRLSFDRVTSGLDSGEAGIAEFSTNGSTYTAVESIRTASGRVTFNLPTSAENQSGLRLRFRINASLSSETYTVDNIRLEGTSGSGGGTTNPFEKGPDPTKTMLEASTGPFTYTTTTVSSTTASGYRQGTIYHPTNVTGPFAAVAVVPGYLASQSSINWWGPRLASHGFVVITIDTNSTSDQPPSRATQLMAALNQLKTFSNTSSHPIYRKVDPNRLGVMGWSMGGGGTLIAARDNPTLKAAIPFAPWNSSTNFSTVSVPTLIIACESDSTAPVNSHASPFYNSLPSTTKKAYLEMNNGSHSCANSGNSNAGLIGKYGVSWMKRFMDNDTRFSPYLCGAPHQADLSLTAIDEYRENCPY

SEQ ID NO: 5 - protein, 304 aa, unidentified
  source 1..304: note = Ideonella sakaiensis
MPFNKKSVLALCGAGALLFSMSALANNPAPTDPGDSGGGSAYQRGPDPSVSFLEADRGQYSVRSSRVSSLVSGFGGGTIYYPTGTTGTMGAVVVIPGFVSAESSIDWWGPKLASYGFVVMTIDTNTGFDQPPSRARQINNALDYLVSQNSRSSSPVRGMIDTNRLGVIGWSMGGGGTLRVASEGRIKAAIPLAPWDTTSYYASRSQAPTLIFACESDVIAPVLQHASPFYNSLPSSIDKAFVEINGGSHYCGNGGSIYNDVLSRFGVSWMKLHLDEDSRYKQFLCGPNHTSDSQISDYRGNCPY

SEQ ID NO: 6 - protein, 292 aa, unidentified
  source 1..292: note = Rhizobacter gummiphilus
MFGKLPFARASLAVGALLLSAAAVAQTNPYQRGPDPTVSSLEATRGPFSTSSFTVSRPSGYGAGTVYYPTNAGGKVGAIAVVPGYTARQSSINWWGPRLASHGFVVITIDTNSTLDQPSSRSSQQMAALRQVVSLAGTSSSPIYNKVDTARLGVMGWSMGGGGSLISAKNNPSLRAAAPQAPWAQESFSSVTVPTLIVSCENDSIAPNSSHSFPFYNQMTRNKKANLVINGGSHSCANSGNSDAGLIGKYGVAWMKRFMDDDTRYSKFLCGAEHQADLSKRAVEAYKENCPY

SEQ ID NO: 7 - protein, 293 aa, unidentified
  source 1..293: note = bacterium HR29
MQVVLGRVRSAGLLAALLALAAWALVWASPSAEAQSNPYQRGPNPTRSALTTDGPFSVATYSVSRLSVSGFGGGVIYYPTGTTLTFGGIAMSPGYTADASSLAWLGRRLASHGFVVIVINTNSRLDFPDSRASQLSAALNYLRTSSPSAVRARLDANRLAVAGHSMGGGATLRISEQIPTLKAGVPLTPWHTDKTFNTPVPQLIVGAEADTVAPVSQHAIPFYQNLPSTTPKVYVELDNATHFAPNSPNAAISVYTISWMKLWVDNDTRYRQFLCNVNDPALSDFRSNNRHCQ

SEQ ID NO: 8 - protein, 186 aa, Streptomyces scabiei
MAACTDIDVVSARGTFEPGTLGFIVGDPVYAALQKKVAGKSLSSYKVNYPADLSPTSAAQGNADLVNHVRSQAASCPNQRFVLVGYSQGANVVDNSIGISSAGAVVGSPIVATLPAALEPRVSAVLLFGNPIRAIGKSVTGTYQSRTIDFCAAGDPVCENGGGDVGAHLGYRANADAAAAFAATKI

SEQ ID NO: 9 - protein, 480 aa, Clostridium botulinum
  source 1..480: note = strain Hall / ATCC 3502 / NCTC 13319 / Type A
MKKNLNKIATIILLVFSMTLTNFSMIVRAAEPKAQGTQKVESSTTKKEVKDAEETIKIPTLEDIDNLIDSAEEVKSEEDINKMPPLKFPVEFPEVNTRSIIGGNNYPIVLVHGFMGFGRDELLGYKYWGGVVDLQEKLNASGHETYTATVGPVSSNWDRACELYAYIVGGTVDYGEAHAKKFKHNRYGRTYPGIYKNISNENKIHLIGHSMGGQTIRTLTQLLSEGSEEEINCGQENISPLFEGGKHWIHSVSTISTPNDGTTLSDLMPAKDLISYTFGVLGTITGKNKLFSSIYDLKLDQWGLKKQNGESQRDYIERVLDSNIWNSTKDIATYDLSTEGAQELNTWVKAQPDVYYFSWTTQATKESILTGHSVAQIGPMNPIFYPTANLMGRYSRNQKDLPIIDKKWFPNDGVVNCISQDGPKLGSNDVIEQYNGGVKIGQWNAMPRIINTDHMDIVGTFGNVKDWYMDYASFLSNLSR

SEQ ID NO: 10 - protein, 308 aa, unidentified
  source 1..308: note = uncultured bacterium
MPITARNTLASLLLASSALLLSGTAFAANPPGGDPDPGCQTDCNYQRGPDPTDAYLEAASGPYTVSTIRVSSLVPGFGGGTIHYPTNAGGGKMAGIVVIPGYLSFESSIEWWGPRLASHGFVVMTIDTNTIYDQPSQRRDQIEAALQYLVNQSNSSSSPISGMVDSSRLAAVGWSMGGGGTLQLAADGGIKAAIALAPWNSSINDFNRIQVPTLIFACQLDAIAPVALHASPFYNRIPNTTPKAFFEMTGGDHWCANGGNIYSALLGKYGVSWMKLHLDQDTRYAPFLCGPNHAAQTLISEYRGNCPY

SEQ ID NO: 11 - protein, 292 aa, Thermomonospora curvata
  source 1..292: note = strain ATCC 19995 / DSM 43183 / JCM 3096 / KCTC 9072 / NBRC 15933 / NCIMB 10081 / Henssen B9
MKRTLKRALSLLPAAALAASALVAASPAQAAANPYQRGPNPTEASITAARGPFNTAEITVSRLSVSGFGGGKIYYPTTTSEGTFGAIAISPGFTAYWSSLEWLGHRLASQGFVVIGIETNTTLDQPDQRGQQLLAALDYLTQRSAVRDRVDASRLAVAGHSMGGGGSLEAAKARTSLKAAIPLAPWNLDKTWPEVRTPTLIIGGELDAVAPVATHSIPFYNSLSNAPEKAYLELDNASHFFPNITNTQMAKYMIAWMKRFIDDDTRYTQFLCPPPSTGLLSDFSDARFTCPM

SEQ ID NO: 12 - protein, 289 aa, Thermomonospora curvata
  source 1..289: note = strain ATCC 19995 / DSM 43183 / JCM 3096 / KCTC 9072 / NBRC 15933 / NCIMB 10081 / Henssen B9
MSLRKSFGLLSATAALVAGLVAAPPAQAAANPYQRGPDPTESLLRAARGPFAVSEQSVSRLSVSGFGGGRIYYPTTTSQGTFGAIAISPGFTASWSSLAWLGPRLASHGFVVIGIETNTRLDQPDSRGRQLLAALDYLTQRSSVRNRVDASRLAVAGHSMGGGGTLEAAKSRTSLKAAIPIAPWNLDKTWPEVRTPTLIIGGELDSIAPVATHSIPFYNSLTNAREKAYLELNNASHFFPQFSNDTMAKFMISWMKRFIDDDTRYDQFLCPPPRAIGDISDYRDTCPHT

SEQ ID NO: 13 - protein, 296 aa, Thermobifida alba
MSVTTPRREASLLSRAVAVAAAAAATVALAAPAQAANPYERGPNPTESMLEARSGPFSVSEERASRLGADGFGGGTIYYPRENNTYGAIAISPGYTGTQSSIAWLGERIASHGFVVIAIDTNTTLDQPDSRARQLNAALDYMLTDASSSVRNRIDASRLAVMGHSMGGGGTLRLASQRPDLKAAIPLTPWHLNKSWRDITVPTLIIGADLDTIAPVSSHSEPFYNSIPSSTDKAYLELNNATHFAPNITNKTIGMYSVAWLKRFVDEDTRYTQFLCPGPRTGLLSDVDEYRSTCPF

SEQ ID NO: 14 - protein, 489 aa, Bacillus subtilis
MTHQIVTTQYGKVKGTTENGVHKWKGIPYAKPPVGQWRFKAPEPPEVWEDVLDATAYGPICPQPSDLLSLSYTELPRQSEDCLYVNVFAPDTPSQNLPVMVWIHGGAFYLGAGSEPLYDGSKLAAQGEVIVVTLNYRLGPFGFLHLSSFNEAYSDNLGLLDQAAALKWVRENISAFGGDPDNVTVFGESAGGMSIAALLAMPAAKGLFQKAIMESGASRTMTKEQAASTSAAFLQVLGINEGQLDKLHTVSAEDSLKAADQLRIAEKENIFQLFFQPALDPKTLPEEPEKAIAEGAASGIPLLIGTTRDEGYLFFTPDSDVHSQETLDAALEYLLGKPLAEKAADLYPRSLESQIHMMTDLLFWRPAVAYASAQSHYAPVWMYRFDWHPKKPPYNKAFHALELPFVFGNLDGLERMAKAEITDEVKQLSHTIQSAWITFAKTGNPSTEAVNWPAYHEETRETLILDSEITIENDPESEKRQKLFPSKGE

SEQ ID NO: 15 - protein, 262 aa, Thermobifida fusca
MANPYERGPNPTDALLEASSGPFSVSEENVSRLSASGFGGGTIYYPRENNTYGAVAISPGYTGTEASIAWLGGRIASHGFVVITIDTITTLDQPDSRAEQLNAALNHMINRASSTVRSRIDSSRLAVMGHSMGGGGTPRLASQRPDLKAAIPLTPWHLNKNRSSVTVPTLIIGADLDTIAPVATHAKPFYNSLPSSISKAYLELDGATHFAPNIPNKIIGKYSVAWLKRFVDNDTRYTQFLCPGPRDGLFGEVEEYCSTCPF

SEQ ID NO: 16 - protein, 262 aa, Thermobifida cellulosilytica
MANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

SEQ ID NO: 17 - protein, 262 aa, Thermobifida cellulosilytica
MANPYERGPNPTDALLEARSGPFSVSEERASRFGADGFGGGTIYYPRENNTYGAVAISPGYTGTQASVAWLGERIASHGFVVITIDTNTTLDQPDSRARQLNAALDYMINDASSAVRSRIDSSRLAVMGHSMGGGGTLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGLFGEVEEYRSTCPF

SEQ ID NO: 18 - protein, 300 aa, Thermobifida alba
MSVTTPRRETSLLSRALRATAAAATAVVATVALAAPAQAANPYERGPNPTESMLEARSGPFSVSEERASRFGADGFGGGTIYYPRENNTYGAIAISPGYTGTQSSIAWLGERIASHGFVVIAIDTNTTLDQPDSRARQLNAALDYMLTDASSAVRNRIDASRLAVMGHSMGGGGTLRLASQRPDLKAAIPLTPWHLNKSWRDITVPTLIIGAEYDTIASVTLHSKPFYNSIPSPTDKAYLELDGASHFAPNITNKTIGMYSVAWLKRFVDEDTRYTQFLCPGPRTGLLSDVEEYRSTCPF

SEQ ID NO: 19 - protein, 228 aa, unidentified
  source 1..228: note = Thermothielavioides terrestris (strain ATCC 38088 / NRRL 8126) (Thielavia terrestris)
MKFLPILCAAGLAAAAPTQPAGEAAVEARQLFSDTANDLENGVSSNCPKVIFICARGSTETGNLGSSVCPEVANGLKNYYPNQLWVQGVGGAYTADLASNALPGGTSTAAMQEAANMFNLAQQKCPNASVAAGGYSQGTAVVAGGIQSLSAAAKDQIKGVVLFGYTQAQQNHDTIPNFPVDKTMIFCAQGDLVCNGTLIVTAAHFSYITNGDASTKGPAWLHEKIGDA

SEQ ID NO: 20 - protein, 293 aa, synthetic construct
  source 1..293: note = Unknown prokaryotic organism
MDGVLWRVRTAALMAALLALAAWALVWASPSVEAQSNPYQRGPNPTRSALTADGPFSVATYTVSRLSVSGFGGGVIYYPTGTSLTFGGIAMSPGYTADASSLAWLGRRLASHGFVVLVINTNSRFDYPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELDNASHFAPNSNNAAISVYTISWMKLWVDNDTRYRQFLCNVNDPALSDFRTNNRHCQ

SEQ ID NO: 21 - protein, 262 aa, Thermobifida halotolerans
MANPYERGPNPTNSSIEALRGPFRVDEERVSRLQARGFGGGTIYYPTDNNTFGAVAISPGYTGTQSSISWLGERLASHGFVVMTIDTNTTLDQPDSRASQLDAALDYMVEDSSYSVRNRIDSSRLAAMGHSMGGGGTLRLAERRPDLQAAIPLTPWHTDKTWGSVRVPTLIIGAENDTIASVRSHSEPFYNSLPGSLDKAYLELDGASHFAPNLSNTTIAKYSISWLKRFVDDDTRYTQFLCPGPSTGWGSDVEEYRSTCPF

SEQ ID NO: 22 - protein, 230 aa, unidentified
  source 1..230: note = Fusarium vanettenii (Neocosmospora pisi)
MKFFALTTLLAATASALPTSNPAQELEARQLGRTTRDDLINGNSASCRDVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSA

SEQ ID NO: 23 - protein, 319 aa, unidentified
  source 1..319: note = Moraxella sp. (strain TA144)
MFIMIKKSELAKAIIVTGALVFSIPTLAEVTLSETTVSSIKSEATVSSTKKALPATPSDCIADSKITAVALSDTRDNGPFSIRTKRISRQSAKGFGGGTIHYPTNASGCGLLGAIAVVPGYVSYENSIKWWGPRLASWGFVVITINTNSIYDDPDSRAAQLNAALDNMIADDTVGSMIDPKRLGAIGWSMGGGGALKLATERSTVRAIMPLAPYHDKSYGEVKTPTLVIACEDDRIAETKKYANAFYKNAIGPKMKVEVNNGSHFCPSYRFNEILLSKPGIAWMQRYINNDTRFDKFLCANENYSKSPRISAYDYKDCP

SEQ ID NO: 24 - protein, 576 aa, unidentified
  source 1..576: note = Melanocarpus albomyces
MKGIAALVTAALLGRAVAAPPEPPTNVVEKRAAPTVEISTGTIVGTTRLATEAFNGIPFALPPVGQLRLKPPVRLNSSLGVFDASGIAPACPQFLADSDSNEFLAQVINTVTGLPFFQKALKISEDCLTINVIRPKGTKAGDKLPVLFWIYGGGFELGWSSMYDGGPLVTNAIGFGKPFIFVAVNYRVAGFGFMPGKEILADGAANLGHLDQRMGLEWVADNIAAFGGDPDKVTIWGESAGAISVLNQMALFDGDHTYKGKPLFRGAIMNSGSIVPADPVDCPKGQEIYDQVVAKAGCAGASDTLACLRELPYEKFLDAANSVPAILSYNSVALSYLPRPDGKVLTKSPDVLIKEGKYAAVPMIIGDQEDEGTLFSLFQPNITNSEKLVTYLKDLFFHGASREQLEGLVDRYPARISAGSPFRTGLLNEIYPGFKRLAAILGDLVFTLTRRVFLEDATRVNPTVPAWSYLASYDYGTPILGTFHGSDLLQVFFGILPNYASRSIQSYYANFVYNLDPNDASGGTSGKSKVAEEWPRWTADDRTLIQFFANRNGYLKDDFRSEAAEYIGAHVDYLHI

SEQ ID NO: 25 - protein, 301 aa, Thermobifida fusca
MAVMTPRRERSSLLSRALRFTAAAATALVTAVSLAAPAHAANPYERGPNPTDALLEARSGPFSVSEERASRFGADGFGGGTIYYPRENNTYGAVAISPGYTGTQASVAWLGKRIASHGFVVITIDTNTTLDQPDSRARQLNAALDYMINDASSAVRSRIDSSRLAVMGHSMGGGGSLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGLFGEVEEYRSTCPF

SEQ ID NO: 26 - protein, 310 aa, Oleispira antarctica
  source 1..310: note = Oleispira antarctica RB-8
MNKSILKKLSFGTSVLLVSMNALSWTPSPTPNPDPIPDPTPCQDDCDFTRGPNPTPSSLEASTGPYSVATRSVASSVSGFGGGTLHYPTNTTGTMGAIAVVPGFLLQESSIDFWGPKLASHGFVVITISANSGFDQPASRATQLGRALDYVINQSNGSNSPISGMVDTTRLGVVGWSMGGGGALQLASGDRLSAAIPIAPWNQGGNRFDQIETPTLVIACENDVVASVNSHASPFYNRIPSTTDKAYLEINGGSHFCANDGGSIGGLLGKYGVSWMKRFIDNDLRYDAFLCGPDHAANRSVSEYRDTCNY

SEQ ID NO: 27 - protein, 304 aa, Saccharomonospora viridis
MRIRRQAGTGARASMARAIGVMTTALAVLVGAVGGVAGAEVSTAQDNPYERGPDPTEDSIEAIRGPFSVATERVSSFASGFGGGTIYYPRETDEGTFGAVAVAPGFTASQGSMSWYGERVASQGFIVFTIDTNTRLDQPGQRGRQLLAALDYLVERSDRKVRERLDPNRLAVMGHSMGGGGSLEATVMRPSLKASIPLTPWNLDKTWGQVQVPTFIIGAELDTIASVRTHAKPFYESLPSSLPKAYMELDGATHFAPNIPNTTIAKYVISWLKRFVDEDTRYSQFLCPNPTDRAIEEYRSTCPY

SEQ ID NO: 28 - protein, 230 aa, unidentified
  source 1..230: note = Fusarium oxysporum f. sp. raphani 54005
MKFSIISTLLAATASALPAGQDAAALEARQLGGSITRNDLANGNSGSCPGVIFIYARGSTESGNLGTLGPRVASKLEAKYGKNGVWIQGVGGAYRATLGDNALPRGTSSAAIREMLGHFSDANQKCPDAVLIAGGYSQGAALAAASVTDVDAGIREKIAGVVLFGYTKNLQNRGKIPSYPEDRTKVFCNTGDLVCTGSLIVAAPHLAYQSAASGAAPEFLIQKADAAGAA

SEQ ID NO: 29 - protein, 302 aa, unidentified
  source 1..302: note = Halopseudomonas bauzanensis
MINKNLSQSLLAMMAAGALLLSSSAFAVNPPTDGPTDPDQAYERGPDPSVAFLEAPTGPHSVRTSRVSGLVSGFGGGTIHYPTGTTGTMAAIVVIPGFVSAESSIEWWGPKLASHGFVVMTIDTNTGFDQPPSRARQINNALDYLVSQNTSRTSPVNGMIDTERLGVIGWSMGGGGTLRVASEGRIKAAIPLAPWDTTRFRGVQAPTLIFACESDLIAPVRSHASPFYNQLPDDIDKAYVEINNGSHYCANGGGLNNDVLSRFGVSWMKRFLDNDTRYSQFLCGPNHESDRNISEYRGNCPY

SEQ ID NO: 30 - protein, 302 aa, unidentified
  source 1..302: note = Pseudomonas saudimassiliensis
MINKKLSQTLLSMAAAGALMFSASVFATNPPTDEPTNPGQSYERGPDPTVAFLEASSGPYSVRTSRVSGLVSGFGGGTIHYPTGTTGTMAAIVVIPGFVSAESSIDWWGPKLASHGFVVMTIDTNTGFDQPPSRARQINNALDYLVDQNSSRTSPVNGMIDTERLGVIGWSMGGGGTLRVASEGRIKAAIPLAPWDTTRFSGVQAPTLIFACESDLIAPVRSHASPFYNQLPNDIDKAYVEINNGSHYCANGGGLNNDVLSRFGVSWMKRFLDNDTRYSQFLCGPRHESDRKISEYRGNCPY

SEQ ID NO: 31 - protein, 298 aa, Vibrio gazogenes
MMNVLTKCKLALGIIAIFFSLPSFAVPCSDCSNGFERGQVPRVDQLESSRGPYSVKTINVSRLARGFGGGTIHYSTESGGQQGIIAVVPGYVSLEGSIKWWGPRLASWGFTVITIDTNTIYDQPDSRASQLSAAIDYVIDKGNDRSSPIYGLVDPNRVGVIGWSMGGGGSLKLATDRKIDAVIPQAPWYLGLSRFSSITSPTMIIACQADVVAPVSVHASRFYNQIPGTTPKAYFEIALGSHFCANTGYPSEDILGRNGVAWMKRFIDKDERYTQFLCGQNFDSSLRVSEYRDNCSYY

SEQ ID NO: 32 - protein, 315 aa, unidentified
  source 1..315: note = Deinococcus maricopensis (strain DSM 21211 / LMG 22137 / NRRL B-23946 / LB-34)
MSNGYSACPAPYDHRGMRKHASLLAALPVLLAACGTTTSTLTTPAVTPQTLSGAATNPYERGPAPTTALLEAARGPYATASTTVPRSSVSDFGGATIYYPTSTADGTFGGVAISPGYTGTQASVAWLGPRLASHGFVVIVIDTLSRYDYPSSRGDQLRAALRYLTTSSAVRTRVDATRLAVMGHSMGGGGALEAAKDNPALKAAIPLTPWNTDKSWPELTTPTLIFGAQNDSVAPVSSHAIPFYTSLASTLPKAYLELRGASHGAPTSTNTTIAKYAVAWLKRFEDADRRYDPFLCPTPAVSTLLSDARSTCPFN

SEQ ID NO: 33 - protein, 342 aa, unidentified
  source 1..342: note = Pseudozyma antarctica (Yeast) (Candida antarctica)
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 34 - protein, 319 aa, Thermobifida fusca
  source 1..319: note = Thermobifida fusca (strain YX)
MPPHAARPGPAQNRRGRAMAVITPRRERSSLLSRALRFTAAAATALVTAVSLAAPAHAANPYERGPNPTDALLEARSGPFSVSEERASRFGADGFGGGTIYYPRENNTYGAVAISPGYTGTQASVAWLGERIASHGFVVITIDTNTTLDQPDSRARQLNAALDYMINDASSAVRSRIDSSRLAVMGHSMGGGGTLRLASQRPDLKAAIPLTPWHLNKNWSSVRVPTLIIGADLDTIAPVLTHARPFYNSLPTSISKAYLELDGATHFAPNIPNKIIGKYSVAWLKRFVDNDTRYTQFLCPGPRDGLFGEVEEYRSTCPF

SEQ ID NO: 35 - protein, 304 aa, Acidovorax delafieldii
  source 1..304: note = Acidovorax delafieldii (Pseudomonas delafieldii)
MHLPRSRWDIPFKEETTMTHHFSVRALLAAGALLASAAVSAQTNPYERGPAPTTSSLEASRGPFSYQSFTVSRPSGYRAGTVYYPTNAGGPVGAIAIVPGFTARQSSINWWGPRLASHGFVVITIDTNSTLDQPDSRSRQQMAALSQVATLSRTSSSPIYNKVDTSRLGVMGWSMGGGGSLISARNNPSIKAAAPQAPWSASKNFSSLTVPTLIIACENDTIAPVNQHADTFYDSMSRNPREFLEINNGSHSCANSGNSNQALLGKKGVAWMKRFMDNDRRYTSFACSNPNSYNVSDFRVAACN

SEQ ID NO: 36 - protein, 201 aa, Pseudomonas pseudoalcaligenes
  source 1..201: note = Pseudomonas pseudoalcaligenes (strain CECT 5344)
MRAWWLSGALALMFWAQGAVAGTLLVVGDSISAAFGLDSRQGWVALLEKRLSEEGFEHSVVNASISGDTSAGGAARLSALLAEHKPELVIIELGGNDGLRGQPPAQLQQNLASMVEQSQQAGAKVLLLGMKLPPNYGVRYTTAFAQVFTDLAEQKQVSLVPFFLEGVGGVPGMMQADGIHPAEAAQEILLDNVWPTLKPML

SEQ ID NO: 37 - protein, 259 aa, unidentified
  source 1..259: note = Compost metagenome, hydrolase gene, clone 3
MENPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHFVSNTPDTTTAKYSIAWLKRFVDNDLRYEQFLCPAPDDFAISEYRATCPF

SEQ ID NO: 38 - protein, 259 aa, unidentified
MANPYERGPDPTESSIEAVRGPFAVAQTTVSRLQADGFGGGTIYYPTDTSQGTFGAVAISPGFTAGQESIAWLGPRIASQGFVVITIDTITRLDQPDSRGRQLQAALDHLRTNSVVRNRIDPNRMAVMGHSMGGGGALSAAANNTSLEAAIPLQGWHTRKNWSSVRTPTLVVGAQLDTIAPVSSHSEAFYNSLPSDLDKAYMELRGASHLVSNTPDTTTAKYSIAWLKRFVDDDLRYEQFLCPAPDDFAISEYRSTCPF

(19) United States
(12) Patent Application Publication
(10) Pub. No.: US 2024/0286178 A1
(43) Pub. Date: Aug. 29, 2024

(54) SYSTEMS AND METHODS FOR ENZYMATIC DEGRADATION OF POLYMERS, INCLUDING COPOLYMERS, WITH A VARIETY OF ENZYMES

(71) Applicants: Protein Evolution Inc., Guilford, CT (US); PARIS SCIENCES ET LETTRES, Paris (FR); Centre National de la Recherche Scientifique, Paris (FR); ECOLE SUPERIEURE DE PHYSIQUE ET DE CHIMIE INDUSTRIELLES DE LA VILLE DE PARIS, Paris (FR); SORBONNE UNIVERSITE, Paris (FR)

(72) Inventors: Ludwik Leibler, Amston, CT (US); François Tournilhac, Paris (FR); Clément Freymond, Issy Les Moulineaux (FR); Hernan Garate, Bures-Sur-Yvette (FR); Louise Bréloy, Malakoff (FR); Andrew Griffiths, Paris (FR); Yannick Rondelez, Paris (FR); Brian Mansaku, Amston, CT (US); Jack Pallis, Glastonbury, CT (US); Benjamin Gibbs, North Haven, CT (US)

(73) Assignees: Protein Evolution Inc, Guilford, CT (US); PARIS SCIENCES ET LETTRES, Paris (FR); Centre National de la Recherche Scientifique, Paris (FR); ECOLE SUPERIEURE DE PHYSIQUE ET DE CHIMIE INDUSTRIELLES DE LA VILLE DE PARIS, Paris (FR); SORBONNE UNIVERSITE, Paris (FR)

(21) Appl. No.: 18/408,268

(22) Filed: Jan. 9, 2024

Related U.S. Application Data
(60) Provisional application No. 63/437,953, filed on Jan. 9, 2023.

(51) Int. Cl. B09B 3/60; C12N 9/18

(52) U.S. Cl. CPC B09B 3/60; C12N 9/18; C12Y 301/01074

(57) ABSTRACT

Systems, methods, and compositions relating to pretreatment and enzymatic degradation of polymeric materials comprising one or more crystallizable polymers or copolymers are generally described. Certain aspects are directed to methods comprising reacting a polymeric material comprising a crystallizable polymer or copolymer with a reactive agent to produce a pretreated polymeric material and exposing the pretreated polymeric material to a polymer-degrading enzyme. In some embodiments, the reactive agent induces chain extension, branching, and/or cross-linking of the crystallizable polymer or copolymer. In some embodiments, the reactive agent induces chain scissions followed by chain extension, branching, and/or cross-linking of the crystallizable polymer or copolymer. In some cases, the methods further comprise a thermal annealing step following the step of reacting the polymeric material comprising the crystallizable polymer or copolymer with the reactive agent and prior to the step of exposing the pretreated polymeric material to the polymer-degrading enzyme. During the thermal annealing step, further chain reactions (e.g., chain scission, extension, branching, and/or cross-linking) may occur.

### RELATED APPLICATIONS

This application claims priority under 35 U.S.C. § 119(e) to U.S. Provisional Patent Application No. 63/437,953, filed Jan. 9, 2023, and entitled “PRETREATMENT AND ENZYMATIC DEGRADATION OF SEMI-CRYSTALLINE POLYMERS,” which is incorporated herein by reference in its entirety for all purposes.

### REFERENCE TO AN ELECTRONIC SEQUENCE LISTING

The contents of the electronic sequence listing (P118370016US03-SEQ-TJO.xml; Size: 50,437 bytes; and Date of Creation: Jan. 9, 2024) is herein incorporated by reference in its entirety.

### TECHNICAL FIELD

Systems, methods, and compositions relating to pretreatment and enzymatic degradation of crystallizable polymers or copolymers are generally described.

### BACKGROUND

Some enzymes can be used to catalyze degradation of polymers and can thus be used to recycle plastic waste. However, known methods of enzymatically degrading polymers may have undesirably low efficiency and throughput, particularly for crystallizable polymers or copolymers. Accordingly, improved methods for degrading crystallizable polymers or copolymers are needed.

### SUMMARY

The present disclosure is related to systems, methods, and compositions relating to pretreatment and enzymatic degradation of crystallizable polymers or copolymers. The subject matter of the present invention involves, in some cases, interrelated products, alternative solutions to a particular problem, and/or a plurality of different uses of one or more systems and/or articles.

One aspect is generally directed to a method of processing a polymeric material comprising a crystallizable polymer. In some embodiments, the method comprises reacting the polymeric material comprising the crystallizable polymer with a reactive agent to produce a pretreated polymeric material. In some embodiments, the method comprises exposing the pretreated polymeric material to a polymer-degrading enzyme.

Another aspect is generally directed to a material configured for enzymatic degradation. In some embodiments, the material comprises a post-consumer and/or post-industrial polymeric material (PC/IPM) exhibiting features characterized by a pretreatment for subsequent enzymatic degradation. In certain embodiments, the PC/IPM comprises at least 50 wt. % of a crystallizable polymer. In certain embodiments, the PC/IPM has a linear shear complex modulus G* of at least 1 kPa when measured at a first measurement temperature 30° C. above a melting temperature Tₘ of the crystallizable polymer and at a first angular frequency of 1.0 rad/s. In certain embodiments, the PC/IPM comprises a plurality of features differing from features of a comparative polymeric material. In some instances, the comparative polymeric material is the crystallizable polymer in virgin form. In certain embodiments, the PC/IPM has a crystallization temperature when cooled from a melt at a rate of 20° C./min that is at least 5° C. lower than a crystallization temperature of the comparative polymeric material when cooled from a melt at the same rate. In certain embodiments, the PC/IPM fast cooled from the melt has a crystallization time when measured at a second measurement temperature 30° C. above the glass transition temperature of the crystallizable polymer that is at least 3 minutes longer than a crystallization time of the comparative polymeric material measured at the second measurement temperature.

Another aspect is generally directed to a material configured for enzymatic degradation. In some embodiments, the material comprises a post-consumer and/or post-industrial polymeric material (PC/IPM) exhibiting features characterized by a pretreatment for subsequent enzymatic degradation. In certain embodiments, the PC/IPM comprises at least 50 wt. % of a crystallizable polymer. In certain embodiments, the PC/IPM has a linear shear complex modulus G* of at least 1 kPa when measured at a first measurement temperature 30° C. above a melting temperature Tₘ of the crystallizable polymer and at a first angular frequency of 1.0 rad/s. In certain embodiments, the PC/IPM comprises a plurality of features differing from features of a comparative polymeric material. In some instances, the comparative polymeric material is a polymeric material that is essentially identical in composition to the PC/IPM but has not been pretreated for subsequent enzymatic degradation. In certain embodiments, the PC/IPM has a crystallization temperature when cooled from a melt at a rate of 20° C./min that is at least 5° C. lower than a crystallization temperature of the comparative polymeric material when cooled from a melt at the same rate. In certain embodiments, the PC/IPM fast cooled from the melt has a crystallization time when measured at a second measurement temperature 30° C. above the glass transition temperature of the crystallizable polymer that is at least 3 minutes longer than a crystallization time of the comparative polymeric material measured at the second measurement temperature.

Another aspect is generally directed to a material configured for enzymatic degradation. In some embodiments, the material comprises a post-consumer and/or post-industrial polymeric material (PC/IPM) exhibiting features characterized by a pretreatment for subsequent enzymatic degradation. In certain embodiments, the PC/IPM comprises at least 50 wt. % of polyethylene terephthalate (PET). In certain embodiments, the PC/IPM has a crystallization temperature less than 199° C. when cooled from a melt at a rate of 20° C./min. In certain embodiments, the PC/IPM has a crystallization time of at least 16 minutes when measured at a temperature 30° C. above a glass transition temperature of PET after fast cooling from the melt. In certain embodiments, the PC/IPM has a heat of crystallization less than 48.5 J/g when cooled from the melt at a rate of 20° C./min. In certain embodiments, the PC/IPM has a linear shear complex modulus G* of at least 1000 Pa when measured at a temperature 30° C. above the melting temperature of PET and at an angular frequency of 1.0 rad/s.

Yet another aspect is generally directed to a polymeric material. In some embodiments, the polymeric material comprises a pretreated polymeric material produced by reacting polyethylene terephthalate with diglycidyl terephthalate. In certain embodiments, a crystallization time of the pretreated polymeric material soaked at 70° C. in phosphate buffer at a given measurement temperature is at least 2 times longer than a crystallization time of polyethylene terephthalate at the given measurement temperature.

Another aspect is generally directed to a method for processing a polymeric material. In some embodiments, a method of processing a polymeric material comprises a crystallizable polymer or copolymer, comprising: exposing a polymeric material to a polymer-degrading enzyme at a temperature of at least 20° C. for a duration of less than or equal to 4 days to obtain a reaction yield, wherein the reaction yield is at least 15%.

Another aspect is generally directed to a method for processing a polymeric material. In some embodiments, a method of processing a polymeric material comprises a crystallizable polymer or copolymer, comprising: exposing a polymeric material to a polymer-degrading enzyme selected from Table 1 to obtain a reaction yield, wherein the reaction yield is at least 15%.

Another aspect is generally directed to a material configured for enzymatic degradation. In some embodiments, a material configured for enzymatic degradation, comprises a post-consumer and/or post-industrial polymeric material (PC/IPM) comprising at least 50 wt. % of a crystallizable polymer or copolymer, wherein the PC/IPM comprises a plurality of particles with an average particle size greater than or equal to 50 micrometers.

Yet another aspect is generally directed to a material configured for enzymatic degradation. In some embodiments, a material configured for enzymatic degradation comprises a plurality of particles of a post-consumer and/or post-industrial polymeric material (PC/IPM) with an average particle size greater than or equal to 50 micrometers, wherein the PC/IPM is pretreated with a reactive agent.

Another aspect is generally directed to a method of processing a polymeric material comprising a crystallizable polymer or copolymer. In some embodiments, a method of processing a polymeric material comprising a crystallizable polymer or copolymer, comprises exposing a polymeric material to a polymer-degrading enzyme, wherein the polymeric material comprises a plurality of particles with an average particle size greater than or equal to 50 micrometers, and wherein the reaction yield obtained after exposure of the polymeric material to the polymer-degrading enzyme is at least 60%.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

FIG. 1A shows a chemical structure of polyethylene terephthalate (PET), according to some embodiments.

FIG. 1B shows a chemical structure of diglycidyl terephthalate (DGT)), according to some embodiments.

FIG. 2A shows esterification of carboxyl end groups, according to some embodiments.

FIG. 2B shows etherification of hydroxyl end groups, according to some embodiments.

FIG. 2C shows formation of chain-extended PET, according to some embodiments.

FIG. 2D shows branching from the secondary hydroxyl groups produced from the reactions shown in FIGS. 2A and 2B, according to some embodiments.

FIG. 3 shows, according to some embodiments, a possible cross-linking reaction during reactive extrusion or reactive mixing of PET with DGT, according to some embodiments.

FIG. 4A shows variation of axial force as a function of reactive extrusion or reactive mixing time for the conditions of Example 1 and Comparative Example 2, according to some embodiments.

FIG. 4B shows variation of axial force as a function of reactive extrusion or reactive mixing time for the conditions of Example 2, Example 3, Example 4, Comparative Example 2, and Example 6, according to some embodiments.

FIG. 4C shows variation of axial force as a function of reactive extrusion or reactive mixing time for the conditions of Example 7 and Comparative Example 3, according to some embodiments.

FIG. 5A shows DSC first heating scans for the different PET samples of Examples 1, 2, 3, 4, 5, and Comparative Example 1, according to some embodiments.

FIG. 5B shows DSC first heating scans for the different recycled PET (rPET) samples of Example 7, Example 8, and Comparative Example 3, according to some embodiments.

FIG. 6A shows rheometry results of linear shear complex modulus G* v. time at an angular frequency of 1 rad·s⁻¹, 0.5% strain and T=280° C. for the PET sample described in Example 1, according to some embodiments.

FIG. 6B shows rheometry results of linear shear complex modulus G* measured at T=280° C. for the PET samples described in Comparative Example 1, Example 1, Example 13, and at the conditions equivalent to Example 5, according to some embodiments.

FIG. 6C shows rheometry results of linear shear complex modulus G* v. time at an angular frequency of 1 rad·s⁻¹, 0.5% strain and T=280° C. for the rPET sample described in Example 7, according to some embodiments.

FIG. 6D shows rheometry results of linear shear complex modulus G* measured at T=280° C. for the rPET samples described in Comparative Example 3, Example 7, Example 14, and at the conditions equivalent of Example 8, according to some embodiments.

FIG. 6E shows rheometry results of linear shear complex modulus G* measured at T=280° C. for the rPET samples of Comparative Example 3, Example 10, Example 15, and at the conditions equivalent of Example 11 and Example 12, according to some embodiments.

FIG. 6F shows rheometry results of storage modulus (G′) and loss modulus (G″) v. time at an angular frequency of 1 rad·s⁻¹, 0.5% strain and T=280° C. for the PET sample described in Example 1, according to some embodiments.

FIG. 6G shows rheometry results of storage modulus (G′) and loss modulus (G″) v. time at an angular frequency of 1 rad·s⁻¹, 0.5% strain and T=280° C. for the rPET sample described in Example 7, according to some embodiments.

FIG. 6H shows rheometry results of storage modulus (G′) and loss modulus (G″) v. time at an angular frequency of 0.1 rad·s⁻¹, 0.5% strain and T=280° C. for the PET samples described in Example 1 and Example 5, according to some embodiments.

FIG. 6I shows rheometry results of storage modulus (G′) and loss modulus (G″) v. time at an angular frequency of 0.1 rad·s⁻¹, 0.5% strain and T=280° C. for the rPET samples described in Example 7 and Example 8, according to some embodiments.

FIG. 7 shows isothermic DSC results of heat flow v. incubation time at 75° C. for the PET samples described in Example 18 and Comparative Example 4, according to some embodiments.

FIG. 8A shows FT-IR spectra of samples obtained by the conditions described in Example 1 and Comparative Example 1, according to some embodiments.

FIG. 8B shows FT-IR spectra of samples obtained by the conditions described in Examples 1 and 5, according to some embodiments.

FIG. 8C shows FT-IR spectra of samples obtained by the conditions described in Example 7, Example 8, and Comparative Example 3, according to some embodiments.

FIG. 9 shows enzymatic depolymerization activity of milled rPET obtained by the conditions described in Example 23, Example 24, and Comparative Example 5 using HiC Novozym at 75° C., according to some embodiments.

FIG. 10 shows enzymatic depolymerization activity of milled PET obtained by the conditions described in Example 25, Example 26, and Comparative Example 6 using an LCC variant at 65° C., according to some embodiments.

FIG. 11A shows enzymatic depolymerization activity of milled rPET obtained by the conditions described in Example 28 and Comparative Example 7 using an LCC variant at 75° C., according to some embodiments.

FIG. 11B shows enzymatic depolymerization activity of milled rPET obtained by the conditions described in Example 29 and Comparative Example 7 using an LCC variant at 75° C., according to some embodiments.

FIG. 11C shows enzymatic depolymerization activity of milled rPET obtained by the conditions described in Example 27 and Comparative Example 7 using an LCC variant at 75° C., according to some embodiments.

FIG. 11D shows enzymatic depolymerization activity of milled rPET obtained by the conditions described in Example 30, Example 31 and Comparative Example 7 using an LCC variant at 75° C., according to some embodiments.

FIG. 12 shows enzymatic depolymerization activity of milled rPET obtained by the conditions described in Example 32, Example 33, and Comparative Example 8 using an LCC variant at 85° C., according to some embodiments.

### DETAILED DESCRIPTION

Systems, methods, and compositions relating to pretreatment and enzymatic degradation of polymeric materials comprising one or more crystallizable polymers are generally described. Certain aspects are directed to methods comprising reacting a polymeric material comprising a crystallizable polymer or copolymer with a reactive agent to produce a pretreated polymeric material and exposing the pretreated polymeric material to a polymer-degrading enzyme. In some embodiments, the reactive agent induces chain extension, branching, and/or cross-linking of the crystallizable polymer or copolymer. In some embodiments, the reactive agent induces chain scissions followed by chain extension, branching, and/or cross-linking of the crystallizable polymer or copolymer. In some cases, the methods further comprise a thermal annealing step following the step of reacting the polymeric material comprising the crystallizable polymer or copolymer with the reactive agent and prior to the step of exposing the pretreated polymeric material to the polymer-degrading enzyme. During the thermal annealing step, further chain reactions (e.g., chain scission, extension, branching, and/or cross-linking) may occur.

Certain aspects are directed to a material configured for enzymatic degradation comprising a post-consumer and/or post-industrial material (PC/IPM), which can be a material or mixture in a recycling stream. In some embodiments, the PC/IPM can comprise at least 50 wt. % of a crystallizable polymer or copolymer and can exhibit features characterized by a pretreatment for subsequent enzymatic degradation. In some cases, the PC/IPM has certain crystallization and/or rheological properties that differ from the corresponding properties of a comparative polymeric material, which can make it more amenable to enzymatic degradation. In some cases, the comparative polymeric material is a virgin polymeric material (e.g., the crystallizable polymer or copolymer in virgin form) or a polymeric material that is essentially identical to the PC/IPM except that it does not exhibit features characterized by the pretreatment (e.g., a PC/IPM precursor that has not undergone the pretreatment).

Such comparative polymeric materials will be simple for those of ordinary skill in the art to identify and/or present for comparison without undue experimentation. The polymeric material (whatever material is used in connection with one or more invention(s) disclosed herein), in virgin form, can be easily obtained. In many cases, the comparative polymeric material is the virgin form of a crystallizable polymer or copolymer that constitutes at least 50% of the PC/IPM. Where the comparative polymeric material is material that is essentially identical to the PC/IPM except that it does not exhibit features characterized by the pretreatment (e.g., a PC/IPM precursor that has not undergone the pretreatment), it can, similarly, be readily obtained. Often, this can be accomplished by preparing a sample post-consumer and/or post-industrial (e.g., recyclable) mixture that is essentially identical in original composition (composition prior to pretreatment) to that of the pretreated polymeric material. “Essentially identical,” in this context, can mean of the same or similar elemental and/or molecular makeup (measured, e.g., via elemental or compositional analysis), and need not be absolutely identical, but can differ in compositional makeup such that the major component's portion in the subject material differs by no more than 20%, 15%, 10%, 5%, or 2% from the major component's portion in the comparative polymeric material. In another set of embodiments, the comparative polymeric material is a mixture in which at least 80%, 85%, 90%, 95%, or 98% of the composition includes components that are in the subject material as well (although the subject material and comparative polymeric material may include small amounts of other material not found in the other). This can involve, e.g., material analysis of the pretreated material, then preparation of a mixture with knowledge of how the pretreated material was constituted prior to pretreatment. In another technique, a mixture of material can be prepared, then separated, one portion being the comparative polymeric material, and the other portion being pretreated for comparison. But in all cases, those of ordinary skill in the art will understand how to formulate a comparative polymeric material, whether simply from testing/observation of a pretreated polymeric material, or by forming a mixture and separating that mixture into comparative and treated (pretreated) polymeric material. And those of ordinary skill will understand that “essentially identical” need not be absolutely identical, but similar enough such that the comparison can be made in the context of this disclosure and its methods and materials.

“Pretreated” materials, or “pretreatment,” will be clearly understood by those of ordinary skill in the art. In many embodiments herein, a pretreated material, or a material that has been subjected to pretreatment, is a material that has been treated in a particular way so that it can later engage in a subsequent interaction or reaction. Those of ordinary skill in the art will understand that a pretreated material need not be actually used in a subsequent interaction or reaction. Additionally, those of ordinary skill in the art will understand that one or more pretreatment steps may be performed after one or more other steps (e.g., grinding or otherwise processing raw plastic waste) and/or before one or more other steps (e.g., enzymatic degradation).

“PC/IPM” is a material or materials the makeup of which will be clearly understood by those of ordinary skill in the art. In typical embodiments, such material or materials are polymers that have been formed for a particular use, such as consumer and/or industrial products or processes, then identified for a subsequent transformation, process, reaction, or interaction, such as recycling. A post-consumer and/or post-industrial polymeric material (PC/IPM) may be or may include a manufacturing or compounding scrap or manufactured objects that were never sold to and/or never used by consumers. Post-consumer and/or post-industrial polymeric materials (post-consumer/industrial polymeric materials; PC/IPMs) have generally been a challenging class of materials to recycle. Typically, PC/IPMs include a myriad of polymeric materials (e.g. polymers and/or polymer-based composites, etc). PC/IPMs are materials the makeup of which will be clearly understood by those of ordinary skill in the art. In one set of embodiments, PC/IPMs are polymeric materials generated by households, and/or by commercial, institutional, and/or industrial entities in their role as end or intermediate users of products which can no longer be used or is undesirable its intended purpose. A PC/IPM can be a polymer material diverted during the manufacturing or commercial process. For example, such materials can be polymers and/or copolymers that have been formed for a particular use, then identified for a subsequent transformation, process, reaction, or interaction, such as recycling.

In some embodiments, PC/IPMs comprise plastic waste or mixed plastic waste comprising crystalline polymers or copolymers, amorphous polymers or copolymers, and/or crystallizable polymers or copolymers. Plastic waste, in certain embodiments, may comprise any of myriad of materials that are in whole or in part a polymeric material that an owner and/or holder discards, intends to discard, or is required to discard. In certain embodiments, PC/IPMs comprise at least a portion of plastic waste. “Plastic waste” is a material the makeup of which will be clearly understood by those of ordinary skill in the art. It is to be understood that wherever “PC/IPM” is used herein, this can include plastic waste. It is also to be understood that wherever “plastic waste” is used herein, this can include PC/IPM.

In some embodiments, the post-consumer and/or post-industrial polymeric material comprises a post-consumer and/or post-industrial recycled (PC/IR) plastic, e.g., a post-consumer and/or post-industrial polymeric material that has been used (and may include contaminates, additives or chain modifiers, chain extenders, processing aids, fillers, etc.) and that is subsequently recycled. “PC/IR” is a material or materials the makeup of which will be clearly understood by those of ordinary skill in the art. In typical embodiments, such material or materials are plastic (e.g., polymers) that have been formed for a particular use, such as consumer and/or industrial products or processes, then identified for a subsequent transformation, process, reaction, or interaction, such as recycling.

In some embodiments, PC/IPMs comprise plastic waste or mixed plastic waste comprising crystalline polymers or copolymers, amorphous polymers or copolymers, and/or crystallizable polymers or copolymers. Plastic waste, in certain embodiments, may comprise any of myriad of materials that are in whole or in part a polymeric material that an owner and/or holder discards, intends to discard, or is required to discard. In certain embodiments, PC/IPMs comprise at least a portion of plastic waste. “Plastic waste” is a material the makeup of which will be clearly understood by those of ordinary skill in the art. It is to be understood that wherever “PC/IPM” is used herein, this can include plastic waste. It is also to be understood that wherever “plastic waste” is used herein, this can include PC/IPM. In some embodiments, PC/IPMs comprise post-consumer and/or post-industrial plastic. Post-consumer and/or post-industrial plastic may comprise at least a portion of plastic, in typical embodiments. Plastics, in this context, can be any of a myriad of materials comprising a polymeric material that can be shaped by flow, molded, or otherwise formed into a structure. “Post-consumer and/or post-industrial plastic” are materials the makeup of which will be clearly understood by those of ordinary skill in the art.

Many post-industrial and post-consumer polymeric materials are crystallizable, e.g., can be semi-crystalline when subjected to certain conditions and/or processes (e.g. temperature, pressure, stress, cooling rates from melt, aging, and/or quenching). These materials may be partially and/or fully amorphous as well, under certain conditions which may be different than the aforementioned conditions. Crystallizable polymers or copolymers can include semi-crystalline polymers or copolymers wherein the semi-crystalline polymers or copolymers comprise at least one or more regions of a crystalline phase. Polymers and/or copolymers that may be considered amorphous can be crystallizable when subjected to the aforementioned conditions and/or processes, and therefore, crystallizable polymers or copolymers may include amorphous polymers or copolymers. Those of ordinary skill in the art understand the meaning of each of these terms. As an example, semi-crystalline materials often exhibit some crystalline behavior, but do not always exhibit such behavior under all conditions. It is to be understood that wherever “crystallizable” is used herein, this can include semi-crystalline materials. It is also to be understood that wherever “semi-crystalline” is used herein, this can include crystallizable materials.

“Virgin polymeric material” is a polymeric material that has been produced from petrochemical feedstock (e.g., crude oil, natural gas) and has not been further processed or used to form a consumer or industrial object or product (e.g., a PC/IPM). Those of ordinary skill in the art will understand that virgin polymeric material may comprise one or more additives (e.g., catalysts). A virgin plastic and/or a virgin polymeric material generally refers to a polymeric material that has been produced directly from petrochemical feedstock (e.g., crude oil, natural gas) and has not been previously used or processed (e.g., processed into a consumer or industrial product, used in an industrial process). In some embodiments, a virgin plastic and/or polymeric material can be produced from at least a portion of biomass feedstock. In some embodiments, virgin polymeric materials comprises crystallizable polymers or copolymers in virgin form. A virgin plastic and/or a virgin polymeric material is a material the makeup of which is well understood by those of ordinary skill in the art. A virgin plastic, in certain cases, may comprise some amount (if any) of additives (e.g., catalysts, antioxidants, unreacted monomers, plasticizers, etc.) and comprise crystallizable polymers or copolymers containing some comonomers. The post-consumer and/or post-industrial polymeric material, in certain cases, may comprise some amount of additives (e.g., polymers, small molecules such as but not limited to processing aids, dyes, antioxidants, pigments, fillers, etc.) incorporated into the virgin plastic. In some cases, the virgin polymeric material comprises one or more additives (e.g., catalysts, dyes, contaminants, lubricants, etc).

Certain materials and/or articles (particles) are described herein as “configured” for a particular use (e.g., material configured for enzymatic degradation, systems configured to implement various methods, etc.). Those of ordinary skill in the art will clearly understand the meaning of “configured” in every instance of such use. Scientists, engineers, and technicians who process materials as described herein know how materials are obtained, selected, collected, sorted, and/or treated (including, optionally, removal of spurious materials), prior to their use in processes described herein.

Crystallizable polymers or copolymers (e.g., a semi-crystalline polymer) are often recalcitrant to enzymatic degradation. Although it was expected to be desirable for enzymatic degradation of crystallizable polymers or copolymers to occur at relatively high temperatures (e.g., above a glass transition temperature T_{g} of the crystallizable polymer or copolymer) at least in part because some polymer-degrading enzymes exhibit higher activity at higher temperatures and because chain mobility in polymers is generally increased at higher temperatures, it was found that enzymatic degradation of untreated crystallizable polymers or copolymers (or polymers subjected to conventional pre-treatment methods) often resulted in undesirably slow reaction rates and low yields at relatively high temperatures. Surprisingly, the inventors have discovered that pretreating a polymeric material comprising a crystallizable polymer or copolymer with a reactive agent (e.g., an agent that induces chain extension, branching, and/or cross-linking of the crystallizable polymer or copolymer) prior to exposing the polymeric material to a polymer-degrading enzyme can advantageously increase enzymatic degradation yield (reaction yield) and/or reaction rate. In some cases, this increase in enzymatic degradation yield (reaction yield) and/or reaction rate may be particularly pronounced at relatively high temperatures. This discovery is surprising to those of skill in the art, as reactive agents that induce chain extension, branching, and/or cross-linking of crystallizable polymers or copolymers are conventionally used for the opposite purpose—to improve the properties of crystallizable polymers or copolymers (e.g., by increasing crystallinity)—rather than to facilitate degradation of crystallizable polymers or copolymers.

Without wishing to be bound by any particular theory, pretreatment of a polymeric material comprising a crystallizable polymer or copolymer (e.g., a semi-crystalline polymer) with a reactive agent that induces chain extension, branching, and/or cross-linking of a crystallizable polymer or copolymer may advantageously decrease the crystallinity degree and slow down or even prevent the crystallization process of the pretreated polymeric material from occurring during an enzymatic degradation reaction. In some cases, slowing down or preventing the crystallization process of a polymer may advantageously allow a polymer-degrading enzyme to have sufficient time to degrade the polymer before the polymer achieves a sufficiently high degree of crystallinity to impede the enzymatic degradation process. Accordingly, it has been recognized, within the context of the present disclosure, that certain embodiments described herein can have a number of advantageous effects, including but not limited to enhancing enzymatic degradation of polymeric materials comprising crystallizable polymers or copolymers (e.g., by increasing reaction rates and/or yields), allowing polymer degradation processes to be continuous rather than batch, and expanding the types of enzymes that may be used to degrade crystallizable polymers or copolymers (e.g., thermophilic enzymes).

In some embodiments, methods of processing a polymeric material comprising a crystallizable polymer or copolymer are described. In certain embodiments, the methods comprise reacting the polymeric material comprising the crystallizable polymer or copolymer with a reactive agent to produce a pretreated polymeric material. In certain embodiments, the methods comprise exposing the pretreated polymeric material to a polymer-degrading enzyme.

According to some embodiments, the crystallizable polymer or copolymer may be any polymer comprising a plurality of crystalline regions and a plurality of amorphous regions. Non-limiting examples of suitable crystallizable polymers or copolymers include polyesters, polyamides, polyolefins, polystyrenes (e.g., syndiotactic polystyrenes), fluoropolymers, polyurethanes, polyether ether ketones, crystallizable thermoplastic polyurethanes, substituted forms of the foregoing, and combinations thereof. In some embodiments, the crystallizable polymer comprises a copolymer (e.g., a polymer comprising more than one type of monomer) capable of crystallization. The copolymer may be a block copolymer, a random copolymer, a gradient copolymer, a grafted copolymer, and/or an alternating copolymer. In certain embodiments, the copolymer is formed from one or more olefin-containing monomers and/or one or more amide-containing monomers (e.g., ethylene vinyl alcohol (EVOH), ethylene vinyl acetate (EVA), polyhexamethylene adipamide/polyhexamethylene terephthalamide copolymer (PA66/6T), polyhexamethylene adipamide/polyhexamethylene isophthalamide copolymer (PA66/61), polyether block amide). In certain embodiments, the copolymer is a fluorinated copolymer (e.g., fluorinated ethylene-propylene (FEP), ethylene tetrafluoroethylene (ETFE), ethylenechlorotrifluoroethylene (ECTFE), tetrafluoroethylene propylene (FEPM)).

Examples of polyesters include, but are not limited to, polyethylene terephthalate (PET), poly(lactic acid) (PLA), poly(L-lactic acid) (PLLA), poly(D-lactic acid) (PDLA), polybutylenesuccinate (PBS), polycaprolactone (PCL), poly(ethylene adipate), polybutylene terephthalate (PBT), and combinations thereof. Examples of polyamides include, but are not limited to, polyamide 6, poly(beta-caprolactam), polycaproamide, polyamide-6,6, poly(hexamethylene adipamide) (PA6,6), poly(11-aminoundecanoamide) (PA11), polydodecanolactam (PA12), poly(tetramethylene adipamide) (PA4,6), poly(pentamethylene sebacamide) (PA6,10), poly(hexamethylene dodecanoamide) (PA6,12), poly(m-xylylencadipamide) (PAMXD6), polyhexamethylene adipamide/polyhexamethylene terephthalamide copolymer (PA66/6T), polyhexamethylene adipamide/polyhexamethylene isophthalamide copolymer (PA66/6I), and combinations thereof. Examples of polyolefins include, but are not limited to, polyethylene (e.g., high-density polyethylene, medium-density polyethylene, linear low-density polyethylene, very-low-density polyethylene, etc.), polypropylene, isotactic polypropylene, syndiotactic polypropylene, and combinations thereof. An example of a fluoropolymer includes, but is not limited to, polyvinylidenefluoride (PVDF). In some embodiments, the crystallizable polymer or copolymer is heterogeneous. That is, it comprises a mixture of polymers having the one or more of the above-referenced chemistries.

The crystallizable polymer or copolymer may have any of a variety of appropriate glass transition temperatures (T_{g}). In some embodiments, the crystallizable polymer or copolymer has a glass transition temperature (T_{g}) of at least −150° C., at least −100° C., at least −50° C., at least −20° C., at least 0° C., at least 20° C., at least 50° C., at least 70° C., at least 75° C., at least 80° C., at least 100° C., at least 150° C., at least 200° C., at least 250° C., or at least 280° C. In certain embodiments, the crystallizable polymer or copolymer has a glass transition temperature (T_{g}) in a range from −150° C. to −100° C., −150° C. to −50° C., −150° C. to 0° C., −150° C. to 50° C., −150° C. to 70° C., −150° C. to 75° C., −150° C. to 80° C., −150° C. to 100° C., −150° C. to 150° C., −150° C. to 200° C., −150° C. to 250° C., −150° C. to 280° C., −100° C. to −50° C., −100° C. to 0° C., −100° C. to 50° C., −100° C. to 70° C., −100° C. to 75° C., −100° C. to 80° C., −100° C. to 100° C., −100° C. to 150° C., −100° C. to 200° C., −100° C. to 250° C., −100° C. to 280° C., −50° C. to 0° C., −50° C. to 50° C., −50° C. to 70° C., −50° C. to 75° C., −50° C. to 80° C., −50° C. to 100° C., −50° C. to 150° C., −50° C. to 200° C., −50° C. to 250° C., −50° C. to 280° C., 0° C. to 50° C., 0° C. to 70° C., 0° C. to 75° C., 0° C. to 80° C., 0° C. to 100° C., 0° C. to 150° C., 0° C. to 200° C., 0° C. to 250° C., 0° C. to 280° C., 50° C. to 70° C., 50° C. to 75° C., 50° C. to 80° C., 50° C. to 100° C., 50° C. to 150° C., 50° C. to 200° C., 50° C. to 250° C., 50° C. to 280° C., 70° C. to 100° C., 70° C. to 150° C., 70° C. to 200° C., 70° C. to 250° C., 70° C. to 280° C., 75° C. to 100° C., 75° C. to 150° C., 75° C. to 200° C., 75° C. to 250° C., 75° C. to 280° C., 80° C. to 100° C., 80° C. to 150° C., 80° C. to 200° C., 80° C. to 250° C., 80° C. to 280° C., 100° C. to 150° C., 100° C. to 200° C., 100° C. to 250° C., 100° C. to 280° C., 150° C. to 200° C., 150° C. to 250° C., 150° C. to 280° C., 200° C. to 250° C., 200° C. to 280° C., or 250° C. to 280° C. As used herein, glass transition temperature refers to the midpoint of the transition region in a heating scan (heat flow or normalized heat flow v. temperature) at a constant heating rate of 10° C./minute. The glass transition temperature of the crystallizable polymer or copolymer may be measured using differential scanning calorimetry (DSC) according to standard TA-309. A sample comprising the crystallizable polymer or copolymer may be cooled from room temperature to a temperature at least 30° C. lower than the glass transition temperature. The temperature may be kept constant for 1 minute, and the sample may then be heated at a constant rate of 10° C./min up to a temperature at least 30° C. higher than the glass transition temperature. The glass transition temperature may be obtained as the midpoint of the transition region in the heating scan (heat flow or normalized heat flow v. temperature). General protocols for determining the glass transition temperature using the TA-309 standard are described in more detail in “Measuring the Glass Transition of Amorphous Engineering Thermoplastics,” by TA Instruments, Inc.

The crystallizable polymer or copolymer may have any of a variety of appropriate crystallinity degrees (CD). In some embodiments, the crystallizable polymer or copolymer has a crystallinity degree (CD) of at least 1%, at least 5%, at least 10%, at least 15%, at least 20%, at least 25%, at least 50%, at least 75%, or at least 90%. In some embodiments, the crystallizable polymer or copolymer has a crystallinity degree in a range from 1% to 5%, 1% to 10%, 1% to 15%, 1% to 20%, 1% to 25%, 1% to 50%, 1% to 75%, 1% to 90%, 5% to 10%, 5% to 15%, 5% to 20%, 5% to 25%, 5% to 50%, 5% to 75%, 5% to 90%, 10% to 15%, 10% to 20%, 10% to 25%, 10% to 50%, 10% to 75%, 10% to 90%, 15% to 20%, 15% to 25%, 15% to 50%, 15% to 75%, 15% to 90%, 20% to 50%, 20% to 75%, 20% to 90%, 25% to 50%, 25% to 75%, 25% to 90%, 50% to 75%, 50% to 90%, or 75% to 90%. Crystallinity degree (CD) is defined according to Equation 1:

$$C D = \frac{( Δ {H}_{melt} - Δ {H}_{c r y s t a l l i z a t i o n}}{Δ {H}_{m e l t}^{°}} \cdot 100 \qquad ( 1 )$$

where ΔHₘₑₗₜ is the normalized enthalpy of melting of the crystallizable polymer or copolymer, ΔH_{crystallization} is the normalized enthalpy of crystallization of the crystallizable polymer or copolymer, and ΔHₘₑₗₜ° is the normalized enthalpy of melting of a fully crystalline or crystallizable polymer or copolymer. ΔHₘₑₗₜ and ΔH_{crystallization} may be measured using differential scanning calorimetry (DSC) as described in Example 9 below. For example, DSC heating scans may be obtained using a calorimeter (e.g., a TA Discovery Q200 calorimeter). A sample comprising the crystallizable polymer or copolymer may be heated from 0° C. to 300° C. at a heating rate of 10° C./min, and ΔHₘₑₗₜ and ΔH_{crystallization} may be obtained from the resulting normalized heat flow v. temperature curve.

In some embodiments, the polymeric material comprising the crystallizable polymer or copolymer is a virgin polymeric material. A virgin polymeric material generally refers to a polymeric material that has been produced directly from petrochemical feedstock (e.g., crude oil, natural gas) and has not been previously used or processed (e.g., processed into a consumer or industrial product, used in an industrial process). In some embodiments, the virgin polymeric material comprises the crystallizable polymer or copolymer in virgin form. In certain instances, the virgin polymeric material comprises virgin polyethylene terephthalate (PET). In some cases, the virgin polymeric material comprises one or more additives (e.g., catalysts).

In some embodiments, the polymeric material comprising the crystallizable polymer or copolymer (e.g., semi-crystalline polymer) comprises a post-consumer and/or post-industrial polymeric material. A post-consumer polymeric material generally refers to a polymeric material that has been used in one or more consumer products (e.g., food and beverage containers, packaging for health and beauty products, clothing, automotive components, etc.). A post-industrial polymeric material generally refers to a polymeric material that has been used in or resulted from one or more industrial products (e.g., a product used in a manufacturing process) and/or industrial processes (e.g., waste from a manufacturing process). In certain embodiments, the post-consumer and/or post-industrial polymeric material comprises one or more additives (e.g., dyes, plasticizers, catalysts, antioxidants). In certain embodiments, the post-consumer and/or post-industrial polymeric material comprises one or more contaminants (e.g., paper fibers, adhesives, other polymers, etc.). In some cases, the post-consumer and/or post-industrial polymeric material is formed by mechanically processing (e.g., grinding, washing, drying, etc.) raw waste from one or more consumer products, industrial products, and/or industrial processes. In some cases, the post-consumer and/or post-industrial material is formed by chemically processing one or more components of raw waste from one or more consumer products, industrial products, and/or industrial processes. In some embodiments, a reactive agent is an agent that induces chain extension, branching, and/or cross-linking of the crystallizable polymer or copolymer. In some embodiments, a reactive agent is an agent that induces chain scission followed or accompanied by chain extension, branching, and/or cross-linking of the crystallizable polymer or copolymer. The reactive agent may be a reactive molecule, a monomer, a comonomer, an oligomer, a polymer, or a mixture of thereof.

In some embodiments, the polymeric material comprising the crystallizable polymer or copolymer comprises one or more catalysts (e.g., a catalyst used to control polymerization reactions). The presence of the one or more catalysts may help to control chain extension and/or branching reactions without addition of any additional catalysts. As an illustrative example, Example 22 shows that certain post-consumer PET flakes contained antimony and titanium, which are known as catalysts of transesterification and esterification reactions.

A reaction between a polymeric material comprising a crystallizable polymer or copolymer and a reactive agent may occur through a variety of mechanisms. In some embodiments, the reactive agent reacts with the crystallizable polymer or copolymer in a transesterification, transcarbamoylation, transalkylation, transamination, siloxane-silanoate exchange, thiol-disulfide exchange, imine amine exchange, vinylogous urethane exchange, olefin metathesis, disulfide metathesis, dioxaborolane metathesis, nitroxide radical coupling, and/or Diels Alder cycloaddition reaction. In certain embodiments, the reactive agent reacts with the crystallizable polymer or copolymer to form dynamic covalent bonds. In some cases, dynamic covalent bonds (which, in some cases, can be achieved by an associative or dissociative mechanism) can advantageously produce chain extension, branching, and/or cross-linking of the crystallizable polymer or copolymer without reducing processability during reactive mixing and/or extrusion.

In certain embodiments, the reactive agent comprises at least one reactive functional group (e.g., a functional group that may undergo a chemical reaction with the crystallizable polymer or copolymer). Non-limiting examples of suitable reactive functional groups include epoxy, glycidyl, anhydride, glyceryl, boronic acid, boronate ester, maleimide, dioxaborolane, thioester, polysulfide, aldehyde, amine, acetoacetate ester, radical (e.g., nitroxide radical), furan, and olefin-containing groups. In some embodiments, the reactive agent comprises one or more, two or more, three or more, four or more, five or more, ten or more, fifteen or more, or twenty or more reactive functional groups. In certain embodiments, the reactive agent comprises one to two, one to three, one to four, one to five, one to ten, one to fifteen, one to twenty, two to four, two to five, two to ten, two to fifteen, two to twenty, three to five, three to ten, three to fifteen, three to twenty, four to ten, four to fifteen, four to twenty, five to ten, five to fifteen, five to twenty, ten to fifteen, ten to twenty, or fifteen to twenty reactive functional groups. In certain embodiments, the reactive agent comprises one, two, three, four, five, ten, fifteen, or twenty reactive functional groups.

In certain embodiments, the reactive agent comprises at least a portion of a repeat unit of a backbone of the crystallizable polymer or copolymer. As a non-limiting, illustrative example, when the crystallizable polymer or copolymer comprises polyethylene terephthalate (PET), the reactive agent may comprise a terephthalate component. In some cases, matching the structure of the reactive agent to at least a portion of the structure of the polymeric backbone of the crystallizable polymer or copolymer may advantageously limit the number of species released during enzymatic degradation of the crystallizable polymer or copolymer.

In some embodiments, the reactive agent is selected from the group consisting of diglycidyl terephthalate (DGT), bisphenol A diglycidyl ether (DGEBA), novolac resin, cycloaliphatic epoxy, diglycidyl benzenedicarboxylate, triglycidyl benzene tricarboxylate, triglycidyl isocyanurate, epoxidized styrene-acrylic copolymer, diglycidyl phthalate, resorcinol diglycidyl ether, tetrabromobisphenol A diglycidyl ether, bisphenol F diglycidyl ether, 3,4-epoxycyclohexylmethyl-3′-4′-epoxycyclohexane carboxylate, tetraglycidyl methylene dianiline, triglycidyl glycerol, poly(glycolic acid), 1,4-butanediol diglycidyl ether, N,N′-bis[3(carbo-2′,3′-epoxypropoxy)phenyl]pyromellitimide, bis(3,4-epoxycyclohexylmethyl)adipate, 3,4-epoxycyclohexylmethyl-3,4-epoxycyclohexylate, 1,4-cyclohexanedimethanol diglycidyl ether, 4,4′-methylene-bisphenyl isocyanate, hexamethylene diisocyanate, 1,6-diisocyanato hexane, poly(phenyl isocyanate-co-formaldehyde), polymeric methylene diphenyl isocyanate, bisphenol-A dicyanate, pyromellitic dianhydride, and trimellitic anhydride. In certain embodiments, the reactive agent is a polyol. In certain embodiments, the reactive agent is an aromatic or non-aromatic polysulfide with epoxy end groups (e.g., Thioplast EPS25). In certain embodiments, the reactive agent is a chain extender. Non-limiting examples of suitable chain extenders include Joncryl® ADR 4400, Joncryl® ADR 4385, and Joncryl® ADR 4468. In certain embodiments, the reactive agent is a maleimide-bearing diaxaborolane. In some embodiments, the reactive agent, in whole or in part, comprises DGT, Araldite PT910, Araldite PT912, and/or tris(oxyranylmethyl) benzene-1,2,4-tricarboxylate. In some embodiments, the reactive agent comprises any of a myriad of combinations of compounds listed in this paragraph (See Example 34 and Comparative Example 10).

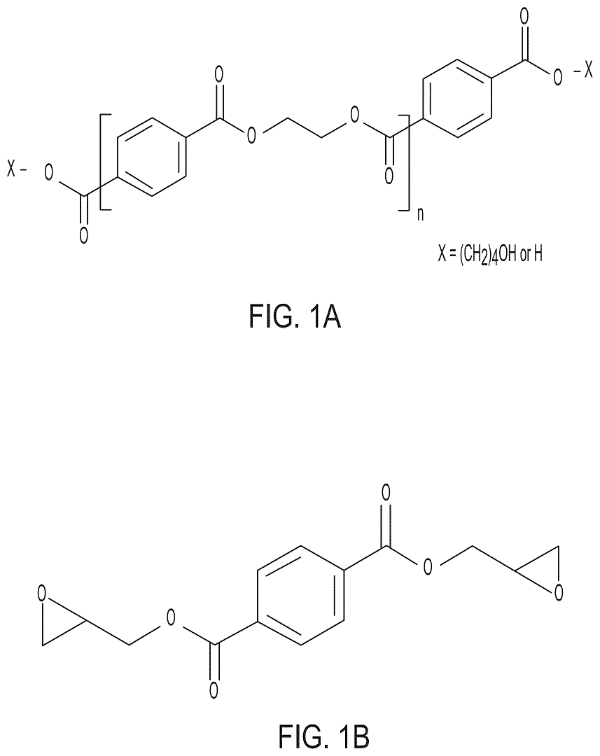

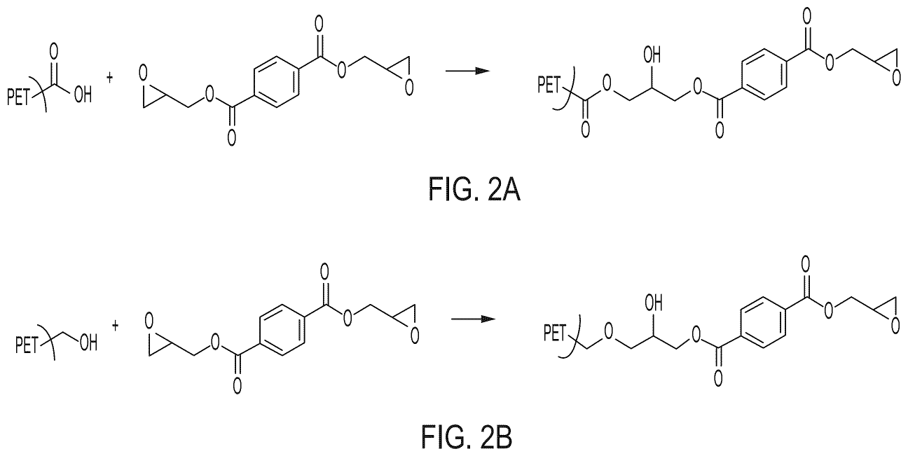

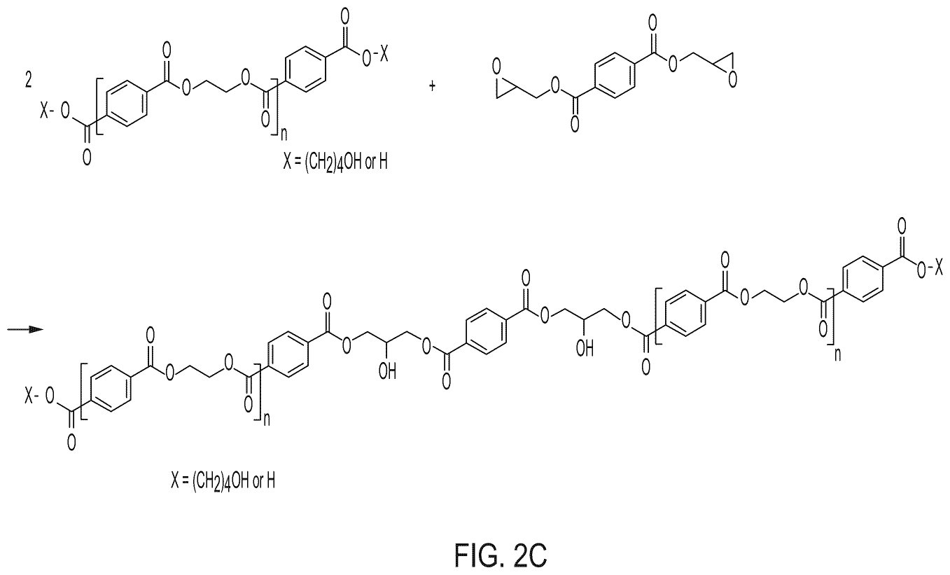

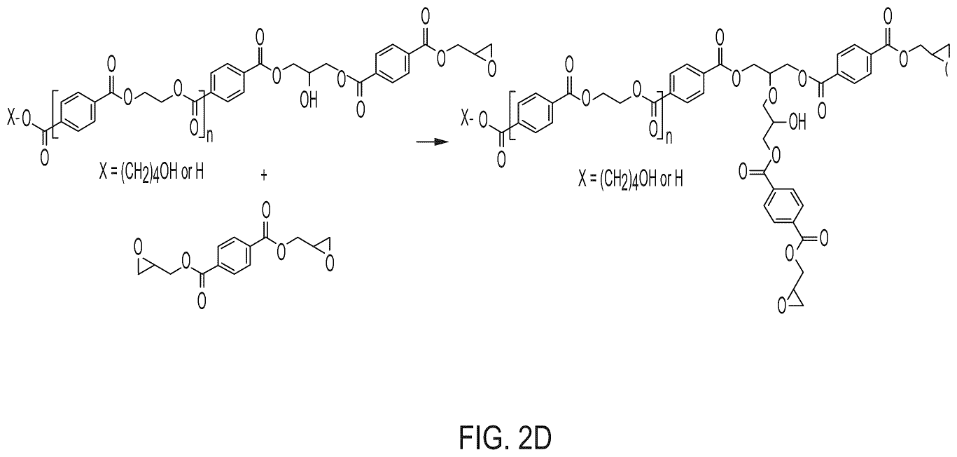

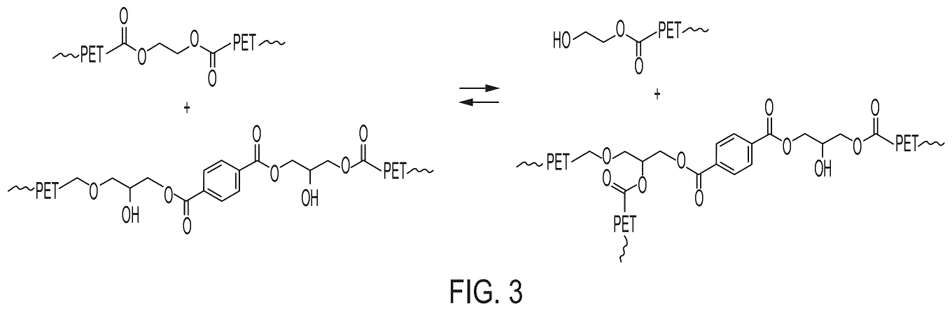

In an illustrative, non-limiting embodiment, a crystallizable polymer or copolymer comprises polyethylene terephthalate (PET) and a reactive agent comprises diglycidyl terephthalate (DGT). A chemical structure of PET is shown in FIG. 1A, and a chemical structure of DGT is shown in FIG. 1B. In some embodiments, a reaction of PET and DGT may result in chain extension and/or branching of PET. For example, FIG. 2A illustrates an exemplary esterification of PET's carboxyl end groups, and FIG. 2B illustrates an exemplary etherification of PET's hydroxyl end groups. FIG. 2C illustrates branching from secondary hydroxyl groups produced from the reactions shown in FIGS. 2A and 2B. FIG. 2D illustrates an exemplary reaction resulting in chain-extended PET. In some embodiments, a reaction of PET and DGT may result in cross-linking of PET. For example, FIG. 3 illustrates an exemplary transesterification reaction resulting in cross-linked PET.

In some embodiments, reacting the polymeric material comprising the crystallizable polymer or copolymer with the reactive agent comprises mixing a mixture of the polymeric material comprising the crystallizable polymer or copolymer and the reactive agent. Mixing the mixture of the polymeric material comprising the crystallizable polymer or copolymer and the reactive agent may be performed according to any method known in the art. In certain embodiments, mixing the mixture of the polymeric material comprising the crystallizable polymer or copolymer and the reactive agent comprises mixing the mixture in a mill, a mixer, and/or a blender.

In some embodiments, a mass content of the reactive agent in the mixture is at least 0.5 wt. %, at least 0.75 wt. %, at least 1 wt. %, at least 1.5 wt. %, at least 2 wt. %, at least 2.5 wt. %, at least 3 wt. %, at least 4 wt. %, at least 5 wt. %, at least 6 wt. %, at least 7 wt. %, at least 8 wt. %, at least 9 wt. %, at least 10 wt. %, at least 15 wt. %, or at least 20 wt. %. In some embodiments, a mass content of the reactive agent in the mixture is in a range from 0.5 wt. % to 1 wt. %, 0.5 wt. % to 2 wt. %, 0.5 wt. % to 3 wt. %, 0.5 wt. % to 4 wt. %, 0.5 wt. % to 5 wt. %, 0.5 wt. % to 10 wt. %, 0.5 wt. % to 15 wt. %, 0.5 wt. % to 20 wt. %, 1 wt. % to 2 wt. %, 1 wt. % to 3 wt. %, 1 wt. % to 4 wt. %, 1 wt. % to 5 wt. %, 1 wt. % to 10 wt. %, 1 wt. % to 15 wt. %, 1 wt. % to 20 wt. %, 2 wt. % to 5 wt. %, 2 wt. % to 10 wt. %, 2 wt. % to 15 wt. %, 2 wt. % to 20 wt. %, 3 wt. % to 5 wt. %, 3 wt. % to 10 wt. %, 3 wt. % to 15 wt. %, 3 wt. % to 20 wt. %, 4 wt. % to 10 wt. %, 4 wt. % to 15 wt. %, 4 wt. % to 20 wt. %, 5 wt. % to 10 wt. %, 5 wt. % to 15 wt. %, 5 wt. % to 20 wt. %, 6 wt. % to 10 wt. %, 6 wt. % to 15 wt. %, 6 wt. % to 20 wt. %, 7 wt. % to 10 wt. %, 7 wt. % to 15 wt. %, 7 wt. % to 20 wt. %, 8 wt. % to 10 wt. %, 8 wt. % to 15 wt. %, 8 wt. % to 20 wt. %, 9 wt. % to 15 wt. %, 9 wt. % to 20 wt. %, 10 wt. % to 15 wt. %, 10 wt. % to 20 wt. %, or 15 wt. % to 20 wt. %.

In some embodiments, the mixture of the polymeric material comprising the crystallizable polymer or copolymer and the reactive agent further comprises one or more additional reagents. In certain embodiments, the one or more additional reagents comprise an antioxidant. A non-limiting example of a suitable antioxidant is Irganox 1010. In certain embodiments, the one or more additional reagents comprise a catalyst. In some cases, the catalyst is a metal catalyst and/or an organic catalyst. A non-limiting example of a suitable catalyst is zinc acetylacetonate.

In some embodiments, a mass content of an additional reagent (e.g., a catalyst, an antioxidant) in the mixture is at least 0.1 wt. %, 0.2 wt. %, 0.5 wt. %, 1 wt. %, 2 wt. %, 5 wt. %, 10 wt. %, 15 wt. %, or 20 wt. %. In some embodiments, a mass content of an additional reagent (e.g., a catalyst, an antioxidant) in the mixture is in a range from 0.1 wt. % to 0.2 wt. %, 0.1 wt. % to 0.5 wt. %, 0.1 wt. % to 1 wt. %, 0.1 wt. % to 2 wt. %, 0.1 wt. % to 5 wt. %, 0.1 wt. % to 10 wt. %, 0.1 wt. % to 15 wt. %, 0.1 wt. % to 20 wt. %, 0.2 wt. % to 0.5 wt. %, 0.2 wt. % to 1 wt. %, 0.2 wt. % to 2 wt. %, 0.2 wt. % to 5 wt. %, 0.2 wt. % to 10 wt. %, 0.2 wt. % to 15 wt. %, 0.2 wt. % to 20 wt. %, 0.5 wt. % to 1 wt. %, 0.5 wt. % to 2 wt. %, 0.5 wt. % to 5 wt. %, 0.5 wt. % to 10 wt. %, 0.5 wt. % to 15 wt. %, 0.5 wt. % to 20 wt. %, 1 wt. % to 5 wt. %, 1 wt. % to 10 wt. %, 1 wt. % to 15 wt. %, 1 wt. % to 20 wt. %, 5 wt. % to 10 wt. %, 5 wt. % to 15 wt. %, 5 wt. % to 20 wt. %, 10 wt. % to 15 wt. %, 10 wt. % to 20 wt. %, or 15 wt. % to 20 wt. %.

In certain embodiments, reacting the polymeric material comprising the crystallizable polymer or copolymer with the reactive agent comprises extruding a mixture of the polymeric material comprising the crystallizable polymer or copolymer and the reactive agent. Extruding the mixture of the polymeric material comprising the crystallizable polymer or copolymer and the reactive agent may be performed using any extruder known in the art. In some embodiments, the extruder is a single screw extruder. In some embodiments, the extruder is a twin screw extruder. The twin screw extruder may be an intermeshing or non-intermeshing twin screw extruder. The intermeshing twin screw extruder may be co-rotating or counter-rotating. In certain embodiments, the twin screw extruder is a conical twin screw extruder. In some cases, dies of an extruder may be chosen to produce an extrudate having a small diameter and/or a relatively thin film to facilitate thermal exchange.

In some embodiments, methods of processing a polymeric material comprising a crystallizable polymer or copolymer further comprise thermally annealing (e.g., isothermally annealing) a mixture of the polymeric material comprising the crystallizable polymer or copolymer and the reactive agent. In some cases, a thermal annealing step may advantageously increase a degree of cross-linking of the crystallizable polymer or copolymer.

In some embodiments, thermally annealing the mixture of the polymeric material comprising the crystallizable polymer or copolymer and the reactive agent comprises heating the mixture to a maximum temperature that is at or above a temperature that is 70° C. lower than, 50° C. lower than, 20° C. lower than, 10° C. lower than, 0° C. lower than, 5° C. higher than, 10° C. higher than, 15° C. higher than, 20° C. higher than, or 50° C. higher than a melting temperature Tₘ of the crystallizable polymer or copolymer. In some embodiments, the maximum temperature of the thermal annealing step is in a range from 70° C. lower than the Tₘ to 50° C. lower than the Tₘ, 70° C. lower than the Tₘ to 20° C. lower than the Tₘ, 70° C. lower than the Tₘ to 10° C. lower than the Tₘ, 70° C. lower than the Tₘ to 0° C. lower than the Tₘ, 70° C. lower than the Tₘ to 5° C. higher than the Tₘ, 70° C. lower than the Tₘ to 10° C. higher than the Tₘ, 70° C. lower than the Tₘ to 15° C. higher than the Tₘ, 70° C. lower than the Tₘ to 20° C. higher than the Tₘ, 70° C. lower than the Tₘ to 50° C. higher than the Tₘ, 50° C. lower than the Tₘ to 20° C. lower than the Tₘ, 50° C. lower than the Tₘ to 10° C. lower than the Tₘ, 50° C. lower than the Tₘ to 0° C. lower than the Tₘ, 50° C. lower than the Tₘ to 5° C. higher than the Tₘ, 50° C. lower than the Tₘ to 10° C. higher than the Tₘ, 50° C. lower than the Tₘ to 15° C. higher than the Tₘ, 50° C. lower than the Tₘ to 20° C. higher than the Tₘ, 50° C. lower than the Tₘ to 50° C. higher than the Tₘ, 20° C. lower than the Tₘ to 10° C. lower than the Tₘ, 20° C. lower than the Tₘ to 0° C. lower than the Tₘ, 20° C. lower than the Tₘ to 5° C. higher than the Tₘ, 20° C. lower than the Tₘ to 10° C. higher than the Tₘ, 20° C. lower than the Tₘ to 15° C. higher than the Tₘ, 20° C. lower than the Tₘ to 20° C. higher than the Tₘ, 20° C. lower than the Tₘ to 50° C. higher than the Tₘ, 10° C. lower than the Tₘ to 0° C. lower than the Tₘ, 10° C. lower than the Tₘ to 5° C. higher than the Tₘ, 10° C. lower than the Tₘ to 10° C. higher than the Tₘ, 10° C. lower than the Tₘ to 15° C. higher than the Tₘ, 10° C. lower than the Tₘ to 20° C. higher than the Tₘ, 10° C. lower than the Tₘ to 50° C. higher than the Tₘ, the Tₘ to 5° C. higher than the Tₘ, the Tₘ to 10° C. higher than the Tₘ, the Tₘ to 15° C. higher than the Tₘ, the Tₘ to 20° C. higher than the Tₘ, the Tₘ to 50° C. higher than the Tₘ, 5° C. higher than the Tₘ to 10° C. higher than the Tₘ, 5° C. to 15° C. higher than the Tₘ, 5° C. to 20° C. higher than the Tₘ, 5° C. to 50° C. higher than the Tₘ, 10° C. to 15° C. higher than the Tₘ, 10° C. to 20° C. higher than the Tₘ, 10° C. to 50° C. higher than the Tₘ, 15° C. to 20° C. higher than the Tₘ, 15° C. to 50° C. higher than the Tₘ, or 20° C. to 50° C. higher than the Tₘ. The melting temperature of the crystallizable polymer or copolymer may be measured using differential scanning calorimetry (DSC) as described in Example 9 below. For example, DSC heating scans may be obtained using a calorimeter (e.g., a TA Discovery Q200 calorimeter). A sample comprising the crystallizable polymer or copolymer may be heated from 0° C. to 300° C. at a heating rate of 10° C./min, and the melting temperature may be obtained from the resulting normalized heat flow v. temperature curve as the peak temperature of the melting signal.

In some embodiments, the maximum temperature of the thermal annealing step is at least 5° C., at least 10° C., at least 15° C., at least 20° C., or at least 50° C. lower than a degradation temperature T_{deg} of the crystallizable polymer or copolymer. In some embodiments, the maximum temperature of the thermal annealing step is 5° C. to 10° C. lower, 5° C. to 15° C. lower, 5° C. to 20° C. lower, 5° C. to 50° C. lower, 10° C. to 15° C. lower, 10° C. to 20° C. lower, 10° C. to 50° C. lower, 15° C. to 20° C. lower, 15° C. to 50° C. lower, or 20° C. to 50° C. lower than the degradation temperature of the crystallizable polymer or copolymer. The degradation temperature T_{deg} of the crystallizable polymer or copolymer may be measured by thermogravimetric analysis (TGA).

In some embodiments, the maximum temperature of the thermal annealing step is at or above a temperature that is 70° C. lower than, 50° C. lower than, 20° C. lower than, 10° C. lower than, 0° C. lower than, 5° C. higher than, 10° C. higher than, 15° C. higher than, 20° C. higher than, or 50° C. higher than a melting temperature Tₘ of the crystallizable polymer or copolymer and is at least 5° C., at least 10° C., at least 15° C., at least 20° C., or at least 50° C. lower than the degradation temperature of the crystallizable polymer or copolymer. In certain embodiments, the maximum temperature of the thermal annealing step is at least 5° C. higher than the melting temperature of the crystallizable polymer or copolymer and at least 5° C. lower than the degradation temperature of the crystallizable polymer or copolymer.

In some embodiments, the maximum temperature of the thermal annealing step is at least 200° C., at least 250° C., at least 255° C., at least 260° C., at least 265° C., at least 270° C., at least 280° C., at least 300° C., at least 350° C., or at least 400° C. In certain embodiments, the maximum temperature of the thermal annealing step is in a range from 200° C. to 250° C., 200° C. to 255° C., 200° C. to 260° C., 200° C. to 265° C., 200° C. to 280° C., 200° C. to 300° C., 200° C. to 350° C., 200° C. to 400° C., 250° C. to 280° C., 250° C. to 300° C., 250° C. to 350° C., 250° C. to 400° C., 255° C. to 280° C., 255° C. to 300° C., 255° C. to 350° C., 255° C. to 400° C., 260° C. to 280° C., 260° C. to 300° C., 260° C. to 350° C., 260° C. to 400° C., 265° C. to 280° C., 265° C. to 300° C., 265° C. to 350° C., 265° C. to 400° C., 280° C. to 300° C., 280° C. to 350° C., 280° C. to 400° C., 300° C. to 350° C., 300° C. to 400° C., or 350° C. to 400° C.

In some embodiments, thermally annealing the mixture of the polymeric material comprising the crystallizable polymer or copolymer and the reactive agent comprises heating the mixture at the maximum temperature for an annealing duration. In certain embodiments, the annealing duration may be adapted to avoid appreciable crystallization (e.g., more than 10%) during annealing. In some embodiments, the annealing duration is at least 10 seconds, at least 30 seconds, at least 1 minute, at least 2 minutes, at least 3 minutes, at least 5 minutes, at least 10 minutes, at least 15 minutes, at least 20 minutes, at least 25 minutes, at least 30 minutes, at least 45 minutes, at least 60 minutes, or at least 90 minutes. In some embodiments, thermally annealing the mixture comprises heating the mixture for a duration in a range from 10 to 30 seconds, 10 seconds to 1 minute, 10 seconds to 2 minutes, 10 seconds to 3 minutes, 10 seconds to 5 minutes, 10 seconds to 10 minutes, 10 seconds to 15 minutes, 10 seconds to 20 minutes, 10 seconds to 25 minutes, 10 seconds to 30 minutes, 10 seconds to 45 minutes, 10 seconds to 60 minutes, 10 seconds to 90 minutes, 30 seconds to 1 minute, 30 seconds to 2 minutes, 30 seconds to 3 minutes, 30 seconds to 5 minutes, 30 seconds to 10 minutes, 30 seconds to 15 minutes, 30 seconds to 20 minutes, 30 seconds to 25 minutes, 30 seconds to 30 minutes, 30 seconds to 45 minutes, 30 seconds to 60 minutes, 30 seconds to 90 minutes, 1 to 3 minutes, 1 to 5 minutes, 1 to 10 minutes, 1 to 15 minutes, 1 to 20 minutes, 1 to 25 minutes, 1 to 30 minutes, 1 to 45 minutes, 1 to 60 minutes, 1 to 90 minutes, 5 to 10 minutes, 5 to 15 minutes, 5 to 20 minutes, 5 to 25 minutes, 5 to 30 minutes, 5 to 45 minutes, 5 to 60 minutes, 5 to 90 minutes, 10 to 15 minutes, 10 to 20 minutes, 10 to 25 minutes, 10 to 30 minutes, 10 to 45 minutes, 10 to 60 minutes, 10 to 90 minutes, 15 to 20 minutes, 15 to 25 minutes, 15 to 30 minutes, 15 to 45 minutes, 15 to 60 minutes, 15 to 90 minutes, 20 to 25 minutes, 20 to 30 minutes, 20 to 45 minutes, 20 to 60 minutes, 20 to 90 minutes, 30 to 60 minutes, 30 to 90 minutes, or 60 to 90 minutes.

In some embodiments, methods of processing a polymeric material comprising a crystallizable polymer or copolymer further comprise slow cooling a mixture of the polymeric material comprising the crystallizable polymer or copolymer and the reactive agent (e.g., via a cooling ramp). In certain embodiments, the slow cooling step comprises cooling the mixture (e.g., from a reactive extrusion or reaction mixing temperature) to a slow cooling temperature at a slow cooling rate. In some instances, the slow cooling temperature is at least 70° C. lower than, at least 50° C. lower than, at least 20° C. lower than, at least 10° C. lower than, or about 0° C. lower than a melting temperature Tₘ of the crystallizable polymer or copolymer. The slow cooling rate may be adapted to avoid appreciable crystallization (e.g., more than 10%) during the slow cooling step. In some instances, the slow cooling step may replace an annealing step. In some instances, the slow cooling step may be followed by an annealing step.

In some embodiments, methods of processing a polymeric material comprising a crystallizable polymer or copolymer further comprise fast cooling a mixture of the polymeric material comprising the crystallizable polymer or copolymer and the reactive agent. The fast cooling step may occur after a reactive extrusion or reactive mixing step, an annealing step, and/or a slow cooling step. In some embodiments, the fast cooling step comprises depositing a product of a prior step of the method (e.g., an extrudate) into a cooling liquid at a fast cooling temperature. In some instances, the cooling liquid comprises water (e.g., ice water). In certain embodiments, the cooling temperature is 25° C. or less, 20° C. or less, 15° C. or less, 10° C. or less, or 5° C. or less. In certain embodiments, the cooling temperature is in a range from 0° C. to 5° C., 0° C. to 10° C., 0° C. to 15° C., 0° C. to 20° C., 0° C. to 25° C., 5° C. to 10° C., 5° C. to 15° C., 5° C. to 20° C., 5° C. to 25° C., 10° C. to 15° C., 10° C. to 20° C., 10° C. to 25° C., 15° C. to 20° C., 15° C. to 25° C., or 20° C. to 25° C.

In some embodiments, the pretreated polymeric material may advantageously comprise a lower glass transition temperature and a lower cold crystallization temperature than a comparative material, wherein the comparative material is the polymeric material prior to pretreatment. Without wishing to be bound by any particular theory, a low cold crystallization temperature may allow for enzymatic degradation to proceed for prolonged periods of time before onset of crystallization, but it alone is not sufficient to achieve relatively high reaction yields. A relatively low glass transition temperature, in addition to a relatively high cold crystallization temperature, may improve the depolymerization rate of the polymer-degrading enzyme by improving enzymatic catalysis. However, low glass transition temperatures are generally associated with fast crystallization rates which, without wishing to be bound by any particular theory, can reduce reaction yield. Accordingly, in some embodiments, the pretreated polymeric material advantageously comprises a relatively low glass transition temperature and a relatively high cold crystallization temperature compared to the polymeric material prior to pretreatment. The aforementioned combination of relative properties may be achieved, in part, by reducing and/or increasing the thermal annealing duration. Solubility and/or rheological tests of pretreated polymeric materials under various thermal annealing durations may carried out to determine the thermal annealing duration that produces a pretreated polymeric material combination of relative properties (See Example 36). As shown in Example 36, the composition of the reactive agent, in some embodiments, can also influence the thermal annealing duration needed to achieve a relatively low glass transition temperature and a relatively high cold crystallization temperature.

In some embodiments, the reactive concentration, the thermal annealing temperature, and/or thermal annealing duration can be controlled to decrease the glass transition temperature and/or increase the cold crystallization of the pretreated polymer. In some embodiments, the reactive agent comprises an amount less than or equal 10 wt. %, less than or equal 5 wt. %, less than or equal 2.5 wt. %, less than or equal 2 wt. %, less than or equal 1.5 wt. %, less than or equal 1 wt. %, or less than or equal 0.5 wt. % of the mixture. In some embodiments, the thermal annealing duration is less than or equal to 1 hour, less than or equal to 40 min, less than or equal to 30 min, less than or equal to 20 min, less than or equal to 10 min, less than or equal to 5 min, or less than or equal to 3 min. In some embodiments, the thermal annealing temperature is greater than or equal to 5° C. higher than the melting temperature of the crystallizable polymer or copolymer and less than or equal to 30° C. higher than the melting temperature of the crystallizable polymer or copolymer. In certain embodiments, using any combination of the amounts reactive agent, durations of thermal annealing, and/or temperatures of thermal annealing listed in the totality of this disclosure, the glass transition of the pretreated polymeric material can be decreased by at least 2° C., at least 5° C., or at least 10° C. and the cold crystallization temperature can be increased at least 2° C., at least 10° C., or at least 20° C. compared to the polymeric material prior to pretreatment. The aforementioned changes in glass transition temperature and cold crystallization temperature may advantageously improve reaction yield upon exposure of the pretreated polymeric material to the polymer-degrading enzyme.

In some embodiments, the PC/IPM comprising the crystallizable polymer or copolymer comprises residual moistures prior to pretreatment. That is, the PC/IPM may not be dried via any of myriad of drying techniques (e.g. ovens, furnaces, dehydrators, etc.) prior to pretreatment. Example 34 depicts PC/IPM that has not undergone a drying process prior to pretreatment. In some embodiments, the lack of drying the PC/IPM before pretreatment can be advantageous due to the complexity and energy consumption of conventional industrial-scale drying operations of PC/IPM.

In some embodiments, the enzymatic degradation of the pretreated polymeric material can occur at relatively low temperatures. In some embodiments, the pretreated polymeric material having a low glass transition temperature and/or a high cold crystallization temperature can be depolymerized with relatively high reaction yields and/or relatively high depolymerization rates when exposed to the polymer-degrading enzyme at relatively low temperatures (e.g. less than or equal to 65° C.). In certain embodiments, the polymer-degrading enzyme produces a maximum reaction yield at relatively low temperatures (e.g. less than or equal to 65° C.). Without wishing to be bound by any particular theory, relatively high reaction yields may be achieved by exposing the pretreated polymeric material, having a relatively high cold crystallization temperature and a relatively low glass transition temperature, to a polymer-degrading enzyme, as the relatively high cold crystallization temperature effectively inhibits blocking reactions (e.g. crystallization) that may occur at higher temperatures and the relatively low glass transition temperature increase enzymatic catalysis. Accordingly, by pretreating the polymeric material, polymer-degrading enzymes that can produce relatively high reaction yields at relatively low temperatures can, unexpectedly, be used to degrade the pretreated polymer material. In certain embodiments, the polymer-degrading enzyme is not a thermophilic enzyme.

In some embodiments, methods of processing a polymeric material comprising a crystallizable polymer or copolymer comprise irradiating a mixture of the polymeric material comprising the crystallizable polymer or copolymer and the reactive agent. In some cases, an irradiating step may advantageously increase a degree of cross-linking of the crystallizable polymer or copolymer. In some embodiments, the irradiating step comprises exposing the mixture of the polymeric material comprising the crystallizable polymer or copolymer and the reactive agent to electron beam irradiation, gamma irradiation, and/or ultraviolet (UV) irradiation.

In some embodiments, methods of processing a polymeric material comprising a crystallizable polymer or copolymer further comprise milling a mixture of the polymeric material comprising the crystallizable polymer or copolymer and the reactive agent to produce a plurality of milled particles of the mixture. In some embodiments, methods of processing the polymeric material comprising the crystallizable polymer or copolymer further comprise selectively isolating a fraction of milled particles of the mixture having a desired particle size.

In some embodiments, the plurality of isolated milled particles of the mixture (e.g., particles of a pretreated polymeric material) comprises relatively large particles. In some cases, it may be possible for polymer-degrading enzymes to degrade pretreated polymeric material at a higher rate than the crystallizable polymer or copolymer. Polymer-degrading enzymes may therefore be able to degrade larger particles of the pretreated polymeric material than of the crystallizable polymer or copolymer. In certain cases, this ability to enzymatically degrade larger particles of the pretreated polymeric material may advantageously reduce the need to achieve smaller particle sizes by milling and/or sorting particles of the pretreated polymeric material. In one particular set of embodiments, the polymer-degrading enzyme may be able to degrade relatively large particles of the pretreated polymeric material having an average particle size greater than or equal to 0.3 mm. In another particular set of embodiments, the polymer-degrading enzyme may be able to degrade particles of the pretreated polymeric material having an average particle size greater than or equal to 0.1 mm. In yet another particular set of embodiments, the polymer-degrading enzyme may be able to degrade particles of the pretreated polymeric material having an average particle size greater than or equal to 25 micrometers.

In some embodiments, the plurality of isolated milled particles of the mixture (e.g., particles of a pretreated polymeric material) has an average particle size of 5 mm or less, 4 mm or less, 3 mm or less, 2 mm or less, 1 mm or less, 600 μm or less, 500 μm or less, 400 μm or less, 300 μm or less, 200 μm or less, 100 μm or less, 50 μm or less, or 25 μm or less. In some embodiments, the plurality of isolated milled particles of the mixture (e.g., particles of a pretreated polymeric material) has an average particle size in a range from 25 μm to 50 μm, 25 μm to 100 μm, 25 μm to 200 μm, 25 μm to 300 μm, 25 μm to 400 μm, 25 μm to 500 μm, 25 μm to 600 μm, 25 μm to 1 mm, 25 μm to 2 mm, 25 μm to 3 mm, 25 μm to 4 mm, 25 μm to 5 mm, 50 μm to 100 μm, 50 μm to 200 μm, 50 μm to 300 μm, 50 μm to 400 μm, 50 μm to 500 μm, 50 μm to 600 μm, 50 μm to 1 mm, 50 μm to 2 mm, 50 μm to 3 mm, 50 μm to 4 mm, 50 μm to 5 mm, 100 μm to 200 μm, 100 μm to 300 μm, 100 μm to 400 μm, 100 μm to 500 μm, 100 μm to 600 μm, 100 μm to 1 mm, 100 μm to 2 mm, 100 μm to 3 mm, 100 μm to 4 mm, 100 μm to 5 mm, 200 μm to 300 μm, 200 μm to 400 μm, 200 μm to 500 μm, 200 μm to 600 μm, 200 μm to 1 mm, 200 μm to 2 mm, 200 μm to 3 mm, 200 μm to 4 mm, 200 μm to 5 mm, 300 μm to 400 μm, 300 μm to 500 μm, 300 μm to 600 μm, 300 μm to 1 mm, 300 μm to 2 mm, 300 μm to 3 mm, 300 μm to 4 mm, 300 μm to 5 mm, 400 μm to 500 μm, 400 μm to 600 μm, 400 μm to 1 mm, 400 μm to 2 mm, 400 μm to 3 mm, 400 μm to 4 mm, 400 μm to 5 mm, 500 μm to 600 μm, 500 μm to 1 mm, 500 μm to 2 mm, 500 μm to 3 mm, 500 μm to 4 mm, 500 μm to 5 mm, 600 μm to 1 mm, 600 μm to 2 mm, 600 μm to 3 mm, 600 μm to 4 mm, 600 μm to 5 mm, 1 mm to 2 mm, 1 mm to 3 mm, 1 mm to 4 mm, 1 mm to 5 mm, 2 mm to 4 mm, 2 mm to 5 mm, 3 mm to 5 mm, or 4 mm to 5 mm. As used herein, the “size” of a particle refers to the maximum distance between two opposed boundaries of an individual particle that can be measured (e.g., a diameter, a length). The “average size” of a plurality of particles refers to the number average of the size of the particles. The average particle size may be determined according to any method known in the art, such as laser diffraction and/or dynamic image analysis.

In some embodiments, the plurality of isolated milled particles of the mixture (e.g., particles of the pretreated polymeric material) has a relatively broad particle size distribution. As noted above, polymer-degrading enzymes may be able to degrade larger particles of a pretreated polymeric material than a crystallizable polymer or copolymer and, therefore, may be able to degrade particles having a broader size distribution than would otherwise be possible without pretreatment. In some embodiments, the standard deviation of particle sizes of the plurality of isolated milled particles of the mixture (e.g., particles of the pretreated polymeric material) is at least 10%, 20%, 30%, 40%, or 50% of the average particle size. In some embodiments, the standard deviation of particle sizes of the plurality of isolated milled particles of the mixture (e.g., particles of the pretreated polymeric material) is in a range from 10% to 20%, 10% to 30%, 10% to 40%, 10% to 50%, 20% to 30%, 20% to 40%, 20% to 50%, 30% to 40%, 30% to 50%, or 40% to 50% of the average particle size. Standard deviation (o) is given its normal meaning in the art and can be calculated according to Equation 2:

$$σ = \sqrt{\frac{{\sum }_{i = 1}^{N} {( {X}_{i} - {X}_{a v g} )}^{2}}{N}} \qquad ( 2 )$$

where Xᵢ is the size of particle i, X_{avg} is the average size of the plurality of particles, and N is the number of particles. The percentage comparisons between the standard deviation and the average particle size outlined above can be obtained by dividing the standard deviation by the average particle size and multiplying by 100%.

In some embodiments, the pretreated polymeric material has a relatively high shear storage modulus G′ and/or shear loss modulus G″. In certain embodiments, a shear storage modulus G′ of the pretreated polymeric material is higher than a shear storage modulus G′ of the crystallizable polymer or copolymer and/or a shear storage modulus G′ of the polymeric material comprising the crystallizable polymer or copolymer (which may, in some cases, comprise post-consumer and/or post-industrial polymeric material). In certain embodiments, a shear loss modulus G″ of the pretreated polymeric material is higher than a shear loss modulus G″ of the crystallizable polymer or copolymer and/or a shear loss modulus G″ of the polymeric material comprising the crystallizable polymer or copolymer (which may, in some cases, comprise post-consumer and/or post-industrial polymeric material). In some cases, pretreatment of a polymeric material comprising a crystallizable polymer or copolymer may induce chain extension, branching, and/or cross-linking of the crystallizable polymer or copolymer, which may lead to an increased shear storage modulus G′ and/or an increased shear loss modulus G″. The shear storage modulus G′ and/or the shear loss modulus G″ of the pretreated polymer, the crystallizable polymer or copolymer, and/or the polymeric material comprising the crystallizable polymer or copolymer may be obtained using a rheometer (e.g., a TA Ares-G2 analyzer). In some cases, for example, the shear storage modulus G′ and/or the shear loss modulus G″ may be measured using the rheometer at a temperature 30° C. above a melting temperature Tₘ of the crystallizable polymer or copolymer, at 0.5% strain, and at an angular frequency of 1.0 rad/s. Those skilled in art will adapt the strain to be in a linear response regime and allow for precise measurement. For example, for rPET in Example 10, the strain is 10%.

In some embodiments, the pretreated polymeric material has a relatively high linear shear complex modulus G*. As used herein, the linear shear complex modulus G* of a material is defined according to Equation 3:

$${G}^{*} = \sqrt{{( {G}^{′} )}^{2} + {( {G}^{″} )}^{2}} \qquad ( 3 )$$

where G′ is the shear storage modulus and G″ is the shear loss modulus of the material. In some embodiments, the pretreated polymeric material has a linear shear complex modulus G* measured at a temperature 30° C. above the melting temperature of the crystallizable polymer or copolymer and at an angular frequency of 1.0 rad/s of at least 0.5 kPa, at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 200 kPa, at least 300 kPa, at least 400 kPa, at least 500 kPa, or at least 1 MPa. In some embodiments, the pretreated polymeric material has a linear shear complex modulus G* measured at a temperature 30° C. above the melting temperature of the crystallizable polymer or copolymer and at an angular frequency of 1.0 rad/s in a range from 0.5 kPa to 1 kPa, 0.5 kPa to 5 kPa, 0.5 kPa to 10 kPa, 0.5 kPa to 15 kPa, 0.5 kPa to 20 kPa, 0.5 kPa to 50 kPa, 0.5 kPa to 100 kPa, 0.5 kPa to 200 kPa, 0.5 kPa to 500 kPa, 0.5 kPa to 1 MPa, 1 kPa to 5 kPa, 1 kPa to 10 kPa, 1 kPa to 15 kPa, 1 kPa to 20 kPa, 1 kPa to 50 kPa, 1 kPa to 100 kPa, 1 kPa to 200 kPa, 1 kPa to 500 kPa, 1 kPa to 1 MPa, 2 kPa to 5 kPa, 2 kPa to 10 kPa, 2 kPa to 15 kPa, 2 kPa to 20 kPa, 2 kPa to 50 kPa, 2 kPa to 100 kPa, 2 kPa to 500 kPa, 2 kPa to 1 MPa, 5 kPa to 10 kPa, 5 kPa to 15 kPa, 5 kPa to 20 kPa, 5 kPa to 50 kPa, 5 kPa to 100 kPa, 5 kPa to 500 kPa, 5 kPa to 1 MPa, 10 kPa to 20 kPa, 10 kPa to 50 kPa, 10 kPa to 100 kPa, 10 kPa to 500 kPa, 10 kPa to 1 MPa, 15 kPa to 50 kPa, 15 kPa to 100 kPa, 15 kPa to 500 kPa, 15 kPa to 1 MPa, 20 kPa to 50 kPa, 20 kPa to 100 kPa, 20 kPa to 500 kPa, 20 kPa to 1 MPa, 50 kPa to 100 kPa, 50 kPa to 500 kPa, 50 kPa to 1 MPa, 100 kPa to 500 kPa, 100 kPa to 1 MPa, or 500 kPa to 1 MPa.

In some embodiments, a linear shear complex modulus G* of the pretreated polymeric material measured at a temperature 30° C. above the melting temperature of the crystallizable polymer or copolymer and at an angular frequency of 1.0 rad/s is higher than a linear shear complex modulus G* of the polymeric material comprising the crystallizable polymer or copolymer measured under the same conditions. In certain embodiments, a linear shear complex modulus G* of the pretreated polymeric material measured at a temperature 30° C. above the melting temperature of the crystallizable polymer or copolymer and at an angular frequency of 1.0 rad/s is at least 40, at least 50, at least 80, at least 100, at least 200, at least 300, at least 400, at least 500, at least 600, at least 700, at least 800, at least 900, at least 1,000, at least 2,000, at least 5,000, at least 8,000, at least 10,000, at least 15,000, at least 20,000, or at least 22,000 times higher than a linear shear complex modulus G* of the polymeric material comprising the crystallizable polymer or copolymer measured under the same conditions. In certain embodiments, a linear shear complex modulus G* of the pretreated polymeric material measured at a temperature 30° C. above the melting temperature of the crystallizable polymer or copolymer and at an angular frequency of 1.0 rad/s is in a range from 40 to 100 times higher, 40 to 200 times higher, 40 to 500 times higher, 40 to 1,000 times higher, 40 to 2,000 times higher, 40 to 5,000 times higher, 40 to 10,000 times higher, 40 to 15,000 times higher, 40 to 20,000 times higher, 40 to 22,000 times higher, 50 to 100 times higher, 50 to 200 times higher, 50 to 500 times higher, 50 to 1,000 times higher, 50 to 2,000 times higher, 50 to 5,000 times higher, 50 to 10,000 times higher, 50 to 15,000 times higher, 50 to 20,000 times higher, 50 to 22,000 times higher, 100 to 200 times higher, 100 to 500 times higher, 100 to 1,000 times higher, 100 to 2,000 times higher, 100 to 5,000 times higher, 100 to 10,000 times higher, 100 to 15,000 times higher, 100 to 20,000 times higher, 100 to 22,000 times higher, 200 to 500 times higher, 200 to 1,000 times higher, 200 to 2,000 times higher, 200 to 5,000 times higher, 200 to 10,000 times higher, 200 to 15,000 times higher, 200 to 20,000 times higher, 200 to 22,000 times higher, 500 to 1,000 times higher, 500 to 2,000 times higher, 500 to 5,000 times higher, 500 to 10,000 times higher, 500 to 15,000 times higher, 500 to 20,000 times higher, 500 to 22,000 times higher, 1,000 to 5,000 times higher, 1,000 to 10,000 times higher, 1,000 to 15,000 times higher, 1,000 to 20,000 times higher, 1,000 to 22,000 times higher, 5,000 to 10,000 times higher, 5,000 to 15,000 times higher, 5,000 to 20,000 times higher, 5,000 to 22,000 times higher, 10,000 to 15,000 times higher, 10,000 to 20,000 times higher, 10,000 to 22,000 times higher, 15,000 to 20,000 times higher, 15,000 to 22,000 times higher, or 20,000 to 22,000 times higher than a linear shear complex modulus G* of the polymeric material comprising the crystallizable polymer or copolymer measured under the same conditions.

In some embodiments, the pretreated polymeric material has a shear storage modulus G′ measured at a temperature 30° C. above the melting temperature of the crystallizable polymer or copolymer and at an angular frequency of 1.0 rad/s of at least 0.5 kPa, at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 500 kPa, or at least 1 MPa. In some embodiments, the pretreated polymeric material has a shear storage modulus G′ measured at a temperature 30° C. above the melting temperature of the crystallizable polymer or copolymer and at an angular frequency of 1.0 rad/s in a range from 1 kPa to 5 kPa, 1 kPa to 10 kPa, 1 kPa to 15 kPa, 1 kPa to 20 kPa, 1 kPa to 50 kPa, 1 kPa to 100 kPa, 1 kPa to 500 kPa, 1 kPa to 1 MPa, 2 kPa to 5 kPa, 2 kPa to 10 kPa, 2 kPa to 15 kPa, 2 kPa to 20 kPa, 2 kPa to 50 kPa, 2 kPa to 100 kPa, 2 kPa to 500 kPa, 2 kPa to 1 MPa, 5 kPa to 10 kPa, 5 kPa to 15 kPa, 5 kPa to 20 kPa, 5 kPa to 50 kPa, 5 kPa to 100 kPa, 5 kPa to 500 kPa, 5 kPa to 1 MPa, 10 kPa to 20 kPa, 10 kPa to 50 kPa, 10 kPa to 100 kPa, 10 kPa to 500 kPa, 10 kPa to 1 MPa, 15 kPa to 50 kPa, 15 kPa to 100 kPa, 15 kPa to 500 kPa, 15 kPa to 1 MPa, 20 kPa to 50 kPa, 20 kPa to 100 kPa, 20 kPa to 500 kPa, 20 kPa to 1 MPa, 50 kPa to 100 kPa, 50 kPa to 500 kPa, 50 kPa to 1 MPa, 100 kPa to 500 kPa, 100 kPa to 1 MPa, or 500 kPa to 1 MPa.

In some embodiments, a shear storage modulus G′ of the pretreated polymeric material measured at a temperature 30° C. above the melting temperature of the crystallizable polymer or copolymer and at an angular frequency of 1.0 rad/s is higher than a shear storage modulus G′ of the polymeric material comprising the crystallizable polymer or copolymer measured under the same conditions. In certain embodiments, a shear storage modulus G′ of the pretreated polymeric material measured at a temperature 30° C. above the melting temperature of the crystallizable polymer or copolymer and at an angular frequency of 1.0 rad/s is at least 50, at least 100, at least 200, at least 300, at least 400, at least 500, at least 600, at least 700, at least 800, at least 900, at least 1,000, at least 2,000, at least 5,000, at least 10,000, at least 15,000, at least 20,000, or at least 22,000 times higher than a shear storage modulus G′ of the polymeric material comprising the crystallizable polymer or copolymer measured under the same conditions. In certain embodiments, a shear storage modulus G′ of the pretreated polymeric material measured at a temperature 30° C. above the melting temperature of the crystallizable polymer or copolymer and at an angular frequency of 1.0 rad/s is in a range from 50 to 100 times higher, 50 to 200 times higher, 50 to 500 times higher, 50 to 1,000 times higher, 50 to 2,000 times higher, 50 to 5,000 times higher, 50 to 10,000 times higher, 50 to 15,000 times higher, 50 to 20,000 times higher, 50 to 22,000 times higher, 100 to 200 times higher, 100 to 500 times higher, 100 to 1,000 times higher, 100 to 2,000 times higher, 100 to 5,000 times higher, 100 to 10,000 times higher, 100 to 15,000 times higher, 100 to 20,000 times higher, 100 to 22,000 times higher, 200 to 500 times higher, 200 to 1,000 times higher, 200 to 2,000 times higher, 200 to 5,000 times higher, 200 to 10,000 times higher, 200 to 15,000 times higher, 200 to 20,000 times higher, 200 to 22,000 times higher, 500 to 1,000 times higher, 500 to 2,000 times higher, 500 to 5,000 times higher, 500 to 10,000 times higher, 500 to 15,000 times higher, 500 to 20,000 times higher, 500 to 22,000 times higher, 1,000 to 5,000 times higher, 1,000 to 10,000 times higher, 1,000 to 15,000 times higher, 1,000 to 20,000 times higher, 1,000 to 22,000 times higher, 5,000 to 10,000 times higher, 5,000 to 15,000 times higher, 5,000 to 20,000 times higher, 5,000 to 22,000 times higher, 10,000 to 15,000 times higher, 10,000 to 20,000 times higher, 10,000 to 22,000 times higher, 15,000 to 20,000 times higher, 15,000 to 22,000 times higher, or 20,000 to 22,000 times higher than a shear storage modulus G′ of the polymeric material comprising the crystallizable polymer or copolymer measured under the same conditions.

In some embodiments, the pretreated polymeric material has a shear loss modulus G″ measured at a temperature 30° C. above the melting temperature of the crystallizable polymer or copolymer and at an angular frequency of 1.0 rad/s of at least 0.5 kPa, at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 500 kPa, or at least 1 MPa. In some embodiments, the pretreated polymeric material has a shear loss modulus G″ measured at a temperature 30° C. above the melting temperature of the crystallizable polymer or copolymer and at an angular frequency of 1.0 rad/s in a range from 1 kPa to 5 kPa, 1 kPa to 10 kPa, 1 kPa to 15 kPa, 1 kPa to 20 kPa, 1 kPa to 50 kPa, 1 kPa to 100 kPa, 1 kPa to 500 kPa, 1 kPa to 1 MPa, 2 kPa to 5 kPa, 2 kPa to 10 kPa, 2 kPa to 15 kPa, 2 kPa to 20 kPa, 2 kPa to 50 kPa, 2 kPa to 100 kPa, 2 kPa to 500 kPa, 2 kPa to 1 MPa, 5 kPa to 10 kPa, 5 kPa to 15 kPa, 5 kPa to 20 kPa, 5 kPa to 50 kPa, 5 kPa to 100 kPa, 5 kPa to 500 kPa, 5 kPa to 1 MPa, 10 kPa to 20 kPa, 10 kPa to 50 kPa, 10 kPa to 100 kPa, 10 kPa to 500 kPa, 10 kPa to 1 MPa, 15 kPa to 50 kPa, 15 kPa to 100 kPa, 15 kPa to 500 kPa, 15 kPa to 1 MPa, 20 kPa to 50 kPa, 20 kPa to 100 kPa, 20 kPa to 500 kPa, 20 kPa to 1 MPa, 50 kPa to 100 kPa, 50 kPa to 500 kPa, 50 kPa to 1 MPa, 100 kPa to 500 kPa, 100 kPa to 1 MPa, or 500 kPa to 1 MPa.

In some embodiments, a shear loss modulus G″ of the pretreated polymeric material measured at a temperature 30° C. above the melting temperature of the crystallizable polymer or copolymer and at an angular frequency of 1.0 rad/s is higher than a shear loss modulus G″ of the crystallizable polymer or copolymer and/or the polymeric material comprising the crystallizable polymer or copolymer measured under the same conditions. In certain embodiments, a shear loss modulus G″ of the pretreated polymeric material measured at a temperature 30° C. above the melting temperature of the crystallizable polymer or copolymer and at an angular frequency of 1.0 rad/s is at least 50, at least 100, at least 200, at least 300, at least 400, at least 500, at least 600, at least 700, at least 800, at least 900, at least 1,000, at least 2,000, at least 5,000, at least 10,000, or at least 16,000 times higher than a shear loss modulus G″ of the polymeric material comprising the crystallizable polymer or copolymer measured under the same conditions. In certain embodiments, a shear loss modulus G″ of the pretreated polymeric material measured at a temperature 30° C. above the melting temperature of the crystallizable polymer or copolymer and at an angular frequency of 1.0 rad/s is in a range from 50 to 100 times higher, 50 to 200 times higher, 50 to 500 times higher, 50 to 1,000 times higher, 50 to 2,000 times higher, 50 to 5,000 times higher, 50 to 10,000 times higher, 50 to 16,000 times higher, 100 to 200 times higher, 100 to 500 times higher, 100 to 1,000 times higher, 100 to 2,000 times higher, 100 to 5,000 times higher, 100 to 10,000 times higher, 100 to 16,000 times higher, 200 to 500 times higher, 200 to 1,000 times higher, 200 to 2,000 times higher, 200 to 5,000 times higher, 200 to 10,000 times higher, 200 to 16,000 times higher, 500 to 1,000 times higher, 500 to 2,000 times higher, 500 to 5,000 times higher, 500 to 10,000 times higher, 500 to 16,000 times higher, 1,000 to 5,000 times higher, 1,000 to 10,000 times higher, 1,000 to 16,000 times higher, 5,000 to 10,000 times higher, 5,000 to 16,000 times higher, or 10,000 to 16,000 times higher than a shear loss modulus G″ of the polymeric material comprising the crystallizable polymer or copolymer measured under the same conditions.

In some embodiments, a pretreated polymeric material (e.g., the crystallizable polymer or copolymer chains of the pretreated polymeric material) has a higher weight average molecular weight than the crystallizable polymer or copolymer and/or the polymeric material comprising the crystallizable polymer or copolymer. In certain embodiments, the pretreated polymeric material has a weight average molecular weight that is at least 5% higher, at least 10% higher, at least 20% higher, at least 50% higher, or at least 80% higher than a weight average molecular weight of the crystallizable polymer or copolymer and/or the polymeric material comprising the crystallizable polymer or copolymer. In certain embodiments, the pretreated polymeric material has a weight average molecular weight that is 5% to 10% higher, 5% to 20% higher, 5% to 50% higher, 5% to 80% higher, 10% to 20% higher, 10% to 50% higher, 10% to 80% higher, 20% to 50% higher, 20% to 80% higher, or 50% to 80% higher than a weight average molecular weight of the crystallizable polymer or copolymer and/or the polymeric material comprising the crystallizable polymer or copolymer. The weight average molecular weight of the pretreated polymeric material, the crystallizable polymer or copolymer, and/or the polymeric material comprising the crystallizable polymer or copolymer may be measured by size exclusion chromatography, dynamic light scattering, and/or rheology in a melt.

In some embodiments, a pretreated polymeric material has a higher intrinsic viscosity than the crystallizable polymer or copolymer and/or the polymeric material comprising the crystallizable polymer or copolymer. In certain embodiments, the pretreated polymeric material has an intrinsic viscosity that is at least 5% higher, at least 10% higher, at least 20% higher, at least 50% higher, or at least 80% higher than an intrinsic viscosity of the crystallizable polymer or copolymer and/or the polymeric material comprising the crystallizable polymer or copolymer. In certain embodiments, the pretreated polymeric material has an intrinsic viscosity that is 5% to 10% higher, 5% to 20% higher, 5% to 50% higher, 5% to 80% higher, 10% to 20% higher, 10% to 50% higher, 10% to 80% higher, 20% to 50% higher, 20% to 80% higher, or 50% to 80% higher than an intrinsic viscosity of the crystallizable polymer or copolymer and/or the polymeric material comprising the crystallizable polymer or copolymer.

In some embodiments, the pretreated polymeric material has a higher gel content than the crystallizable polymer or copolymer and/or the polymeric material comprising the crystallizable polymer or copolymer. In some embodiments, the gel content of the pretreated polymeric material is at least 1%, at least 2%, at least 5%, at least 10%, at least 20%, or at least 50% higher than the gel content of the crystallizable polymer or copolymer and/or the polymeric material comprising the crystallizable polymer or copolymer. In certain embodiments, the pretreated polymeric material has a gel content that is 1% to 2% higher, 1% to 5% higher, 1% to 10% higher, 1% to 20% higher, 1% to 50% higher, 2% to 5% higher, 2% to 10% higher, 2% to 20% higher, 2% to 50% higher, 5% to 10% higher, 5% to 20% higher, 5% to 50% higher, 10% to 20% higher, 10% to 50% higher, or 20% to 50% higher than a gel content of the crystallizable polymer or copolymer and/or the polymeric material comprising the crystallizable polymer or copolymer. Gel content of a material may be measured by separating a soluble fraction and an insoluble fraction of the material (e.g., by long dissolution followed by filtration or by using a Soxhlet), with gel content corresponding to the dry weight fraction.

In some embodiments, a pretreated polymeric material has a longer crystallization time (e.g., the total length of time it takes to complete the crystallization process or the time at which the maximum heat flux is achieved in a DSC trace) than the crystallizable polymer or copolymer and/or the polymeric material comprising the crystallizable polymer or copolymer at a given measurement temperature (e.g., a temperature 30° C. above the glass transition temperature of the crystallizable polymer or copolymer, a temperature 5° C. above the glass transition temperature of the crystallizable polymer or copolymer). In some cases, a longer crystallization time may advantageously delay and/or prevent crystallization during enzymatic degradation.

In certain embodiments, the pretreated polymeric material (e.g., a pretreated polymeric material fast cooled from a melt) has a crystallization time at a measurement temperature 30° C. above the glass transition temperature of the crystallizable polymer or copolymer that is at least 1.1 times, at least 2 times, at least 5 times, at least 8 times, or at least 10 times longer than a crystallization time of the polymeric material comprising the crystallizable polymer or copolymer at the same measurement temperature and measured using the same procedure. In certain embodiments, the pretreated polymeric material has a crystallization time at a measurement temperature 30° C. above the glass transition temperature of the crystallizable polymer or copolymer that is 1.1 to 2 times, 1.1 to 5 times, 1.1 to 8 times, 1.1 to 10 times, 2 to 5 times, 2 to 8 times, 2 to 10 times, 5 to 8 times, 5 to 10 times, or 8 to 10 times longer than a crystallization time of the polymeric material comprising the crystallizable polymer or copolymer at the same measurement temperature. In certain embodiments, the pretreated polymeric material has a crystallization time measured at a measurement temperature 30° C. above the glass transition temperature of the crystallizable polymer or copolymer that is at least 3 minutes, at least 5 minutes, at least 10 minutes, at least 15 minutes, at least 30 minutes, at least 60 minutes, at least 120 minutes, at least 180 minutes, at least 240 minutes, at least 300 minutes, at least 360 minutes, at least 420 minutes, at least 480 minutes, at least 540 minutes, or at least 600 minutes longer than a crystallization time of the polymeric material comprising the crystallizable polymer or copolymer at the same measurement temperature. In some embodiments, the pretreated polymeric material has a crystallization time at a measurement temperature 30° C. above the glass transition temperature of the crystallizable polymer or copolymer that is longer than a crystallization time of the polymeric material comprising the crystallizable polymer or copolymer at the same measurement temperature by 3 to 5 minutes, 3 to 10 minutes, 3 to 15 minutes, 3 to 30 minutes, 3 to 60 minutes, 3 to 120 minutes, 3 to 180 minutes, 3 to 240 minutes, 3 to 300 minutes, 3 to 360 minutes, 3 to 420 minutes, 3 to 480 minutes, 3 to 540 minutes, 3 to 600 minutes, 5 to 10 minutes, 5 to 15 minutes, 5 to 30 minutes, 5 to 60 minutes, 5 to 120 minutes, 5 to 180 minutes, 5 to 240 minutes, 5 to 300 minutes, 5 to 360 minutes, 5 to 420 minutes, 5 to 480 minutes, 5 to 540 minutes, 5 to 600 minutes, 10 to 15 minutes, 10 to 30 minutes, 10 to 60 minutes, 10 to 120 minutes, 10 to 180 minutes, 10 to 240 minutes, 10 to 300 minutes, 10 to 360 minutes, 10 to 420 minutes, 10 to 480 minutes, 10 to 540 minutes, 10 to 600 minutes, 30 to 60 minutes, 30 to 120 minutes, 30 to 180 minutes, 30 to 240 minutes, 30 to 300 minutes, 30 to 360 minutes, 30 to 420 minutes, 30 to 480 minutes, 30 to 540 minutes, 30 to 600 minutes, 60 to 120 minutes, 60 to 180 minutes, 60 to 240 minutes, 60 to 300 minutes, 60 to 360 minutes, 60 to 420 minutes, 60 to 480 minutes, 60 to 540 minutes, 60 to 600 minutes, 120 to 180 minutes, 120 to 240 minutes, 120 to 300 minutes, 120 to 360 minutes, 120 to 420 minutes, 120 to 480 minutes, 120 to 540 minutes, 120 to 600 minutes, 180 to 240 minutes, 180 to 300 minutes, 180 to 360 minutes, 180 to 420 minutes, 180 to 480 minutes, 180 to 540 minutes, 180 to 600 minutes, 240 to 300 minutes, 240 to 360 minutes, 240 to 420 minutes, 240 to 480 minutes, 240 to 540 minutes, 240 to 600 minutes, 300 to 360 minutes, 300 to 420 minutes, 300 to 480 minutes, 300 to 540 minutes, 300 to 600 minutes, 360 to 420 minutes, 360 to 480 minutes, 360 to 540 minutes, 360 to 600 minutes, 420 to 480 minutes, 420 to 540 minutes, 420 to 600 minutes, 480 to 540 minutes, 480 to 600 minutes, or 540 to 600 minutes. The crystallization time may be measured using isothermal differential scanning calorimetry (DSC), with heat flow being monitored as a function of incubation time at the measurement temperature. Additional details regarding measurement of crystallization time are described with respect to Comparative Example 4 and Example 18.

In some embodiments, the pretreated polymeric material has a lower crystallization temperature when cooled from a melt (e.g., at a rate of 20° C./min) than the crystallizable polymer or copolymer and/or the polymeric material comprising the crystallizable polymer or copolymer. In some cases, a lower crystallization temperature may advantageously delay and/or prevent crystallization during enzymatic degradation. In some embodiments, the pretreated polymeric material has a crystallization temperature when cooled from a melt (e.g., at a rate of 20° C./min) that is at least 1° C., at least 2° C., at least 3° C., at least 4° C., at least 5° C., at least 6° C., at least 7° C., at least 8° C., at least 9° C., at least 10° C., at least 15° C., or at least 20° C. lower than a crystallization temperature of the crystallizable polymer or copolymer and/or the polymeric material comprising the crystallizable polymer or copolymer when cooled from a melt (e.g., at a rate of 20° C./min). In some embodiments, the pretreated polymeric material has a crystallization temperature when cooled from melt (e.g., at a rate of 20° C./min) that is in a range from 1° C. to 5° C., 1° C. to 10° C., 1° C. to 15° C., 1° C. to 20° C., 5° C. to 10° C., 5° C. to 15° C., 5° C. to 20° C., 10° C. to 15° C., 10° C. to 20° C., or 15° C. to 20° C. lower than a crystallization temperature of the crystallizable polymer or copolymer and/or the polymeric material comprising the crystallizable polymer or copolymer when cooled from a melt (e.g., at a rate of 20° C./min). The crystallization temperature may be measured using differential scanning calorimetry (DSC). For example, DSC heating scans may be obtained using a calorimeter (e.g., a TA Discovery Q200 calorimeter). A sample comprising the pretreated polymeric material, the crystallizable polymer or copolymer, and/or the polymeric material comprising the crystallizable polymer or copolymer may be heated from 0° C. to 300° C. at a heating rate of 10° C./min, and the crystallization temperature may be obtained from the resulting normalized heat flow v. temperature curve. Additional details regarding measurement of crystallization temperature are described with respect to Example 9.

In some embodiments, the pretreated polymeric material has a lower heat of crystallization when cooled from a melt (e.g., at a rate of 20° C./min) than the crystallizable polymer or copolymer and/or the polymeric material comprising the crystallizable polymer or copolymer. In some embodiments, a heat of crystallization of the pretreated polymeric material when cooled from a melt (e.g., at a rate of 20° C./min) is at least 5%, at least 10%, at least 15%, at least 20%, at least 30%, at least 40%, or at least 50% lower than a heat of crystallization of the crystallizable polymer or copolymer and/or the polymeric material comprising the crystallizable polymer or copolymer when cooled from a melt (e.g., at a rate of 20° C./min). In some embodiments, a heat of crystallization of the pretreated polymeric material when cooled from a melt (e.g., at a rate of 20° C./min) is 5 to 10%, 5 to 15%, 5 to 20%, 5 to 30%, 5 to 40%, 5 to 50%, 10 to 15%, 10 to 20%, 10 to 30%, 10 to 40%, 10 to 50%, 15 to 20%, 15 to 30%, 15 to 40%, 15 to 50%, 20 to 30%, 20 to 40%, 20 to 50%, 30 to 40%, 30 to 50%, or 40 to 50% lower than a heat of crystallization of the crystallizable polymer or copolymer and/or the polymeric material comprising the crystallizable polymer or copolymer when cooled from a melt (e.g., at a rate of 20° C./min). In some embodiments, the heat of crystallization may be measured using DSC. For example, a sample may be heated from 0° C. to 300° C. at a heating rate of 10° C./min in a calorimeter (e.g., a TA Discovery Q200 calorimeter), and the heat of crystallization may be obtained from the resulting normalized heat flow v. temperature curve.

In some embodiments, the pretreated polymeric material has a lower melt mass-flow rate (MFR) than the crystallizable polymer or copolymer and/or the polymeric material comprising the crystallizable polymer or copolymer. The melt mass-flow rate generally refers to the case of flow of a melted material. In some cases, a relatively low melt mass-flow rate may be indicative of increased crosslinking, branching, and/or extension. In some embodiments, the pretreated polymeric material has a melt mass-flow rate measured at a given measurement temperature (e.g., 30° C. above the melting temperature of the crystallizable polymer or copolymer) that is at least 3 times lower, at least 5 times lower, at least 8 times lower, at least 10 times lower, at least 15 times lower, or at least 20 times lower than a mass melt-flow rate of the crystallizable polymer or copolymer and/or the polymeric material comprising the crystallizable polymer or copolymer at the given measurement temperature. In some embodiments, a melt mass-flow rate of the pretreated polymeric material measured at a given measurement temperature (e.g., 30° C. above the melting temperature of the crystallizable polymer or copolymer) is 3 to 5 times lower, 3 to 10 times lower, 3 to 15 times lower, 3 to 20 times lower, 5 to 10 times lower, 5 to 15 times lower, 5 to 20 times lower, 10 to 15 times lower, 10 to 20 times lower, or 15 to 20 times lower than a melt mass-flow rate of the crystallizable polymer or copolymer and/or the polymeric material comprising the crystallizable polymer or copolymer at the given measurement temperature.

In some embodiments, the pretreated polymeric material does not flow. In certain instances, for example, a pretreated polymeric material that has undergone annealing may not flow.

In some embodiments, methods of processing a polymeric material comprising a crystallizable polymer or copolymer (e.g., semi-crystalline polymer) comprise exposing the pretreated polymeric material to a polymer-degrading enzyme. In some embodiments, the polymer-degrading enzyme is a thermostable and/or thermophilic enzyme. In some embodiments, the polymer-degrading enzyme comprises a hydrolase, an esterase, a protease (e.g., a serine protease), a cutinase, a lipase, an oxidase, a peroxidase, and/or an amidase.

Examples of such polymer-degrading enzymes that are useful in methods and compositions provided herein are described in the following US, foreign, and international patents and patent application publications which are incorporated herein by reference in their entirety for all purposes: Japanese Patent No. 5,850,342 entitled “A novel esterase derived from twig leaf compost;” U.S. Pat. No. 11,414,651 entitled “Esterases and uses thereof;” Chinese Patent No. 113584057 entitled “ICCG expression element, expression vector, Bacillus subtilis recombinant strain and method for degrading PET or monomer thereof;” Chinese Patent No. 113684196 entitled “Purification method of high-temperature-resistant polyethylene terephthalate hydrolase;” U.S. Pat. No. 10,590,401 entitled “Esterases and uses thereof;” U.S. Pat. No. 11,535,832 entitled “Esterases and uses thereof;” U.S. Pat. No. 11,692,181 entitled “Esterases and uses thereof;” U.S. Pat. No. 10,584,320 entitled “Esterases and uses thereof;” U.S. Pat. No. 11,072,784 entitled “Esterases and uses thereof;” U.S. Pat. No. 6,995,005 entitled “DNA Sequences Coding for Ester-Group-Cleaving Enzymes;” Chinese Patent No. 101168735 entitled “High-temperature cutinase and gene order thereof;” U.S. application Ser. No. 13/517,331 entitled “Detergent Compositions Containing Thermobifida Fusca Lipase and Methods of use Thereof;” U.S. Pat. No. 11,773,383 entitled “Methods for Promoting Extracellular Expression of Proteins in Bacillus Subtilis Using a Cutinase;” U.S. patent application Ser. No. 14/237,846 entitled “Compositions and Methods Comprising a Lipolytic Enzyme Variant;” U.S. patent application Ser. No. 14/366,165 entitled “Compositions and Methods Comprising a Lipolytic Enzyme Variant;” International Application No. PCT/EP2021/079783 entitled “Novel esterases and their use;” EP3517608 entitled “New Polypeptides Having a Polyester Degrading Activity and Uses Thereof;” U.S. patent application Ser. No. 17/291,291 entitled “Method for the Enzymatic Degradation of Polyethylene Terephthalate;” U.S. patent application Ser. No. 17/625,783 entitled “Esterases And Uses Thereof;” International Application No. PCT/US2023/062092 entitled “Leaf-Branch Compost Cutinase Mutants;” U.S. Pat. No. 6,960,459 entitled “Fungal cutinase for use in the processing of textiles;” U.S. Pat. No. 9,476,072 entitled “Cutinase variants and polynucleotides encoding same;” U.S. Pat. No. 7,943,336 entitled “Cutinase for detoxification of feed products;” and U.S. Pat. No. 9,951,299 entitled “Cutinase variants and polynucleotides encoding same.”

Further examples of polymer-degrading enzymes that are useful in methods and compositions provided herein are described in the following literary publications which are incorporated herein by reference in their entirety for all purposes: Sulaiman S, Yamato S, Kanaya E, Kim J J, Koga Y, Takano K, Kanaya S. Isolation of a novel cutinase homolog with polyethylene terephthalate-degrading activity from leaf-branch compost by using a metagenomic approach. Appl Environ Microbiol. 2012 March; 78(5): 1556-62. doi: 10.1128/AEM.06725-11. Epub 2011 Dec. 22. PMID: 22194294; PMCID: PMC3294458; Tournier, V., Topham, C. M., Gilles, A. et al. An engineered PET depolymerase to break down and recycle plastic bottles. Nature 580, 216-219 (2020). https://doi.org/10.1038/s41586-020-2149-4; Then J, Wei R, Oeser T, Barth M, Belisario-Ferrari M R, Schmidt J, Zimmermann W. Ca2+ and Mg2+ binding site engineering increases the degradation of polyethylene terephthalate films by polyester hydrolases from Thermobifida fusca. Biotechnol J. 2015 April; 10(4):592-8. doi: 10.1002/biot.201400620. Epub 2015 Jan. 19. PMID: 25545638; Sonnendecker C, Oeser J, Richter P K, Hille P, Zhao Z, Fischer C, Lippold H, Blázquez-Sánchez P, Engelberger F, Ramírez-Sarmiento C A, Oeser T, Lihanova Y, Frank R, Jahnke H G, Billig S, Abel B, Sträter N, Matysik J, Zimmermann W. Low Carbon Footprint Recycling of Post-Consumer PET Plastic with a Metagenomic Polyester Hydrolase. ChemSusChem. 2022 May 6; 15(9):e202101062. doi: 10.1002/cssc.202101062. Epub 2022 Feb. 10. PMID: 34129279; PMCID: PMC9303343; Pfaff L, Gao J, Li Z, Jäckering A, Weber G, Mican J, Chen Y, Dong W, Han X, Feiler C G, Ao Y F, Badenhorst C P S, Bednar D, Palm G J, Lammers M, Damborsky J, Strodel B, Liu W, Bornscheuer U T, Wei R. Multiple Substrate Binding Mode-Guided Engineering of a Thermophilic PET Hydrolase. ACS Catal. 2022 Aug. 5; 12(15):9790-9800. doi: 10.1021/acscatal.2c02275. Epub 2022 Jul. 27. PMID: 35966606; PMCID: PMC9361285; Richter, P. K., Blázquez-Sánchez, P., Zhao, Z. et al. Structure and function of the metagenomic plastic-degrading polyester hydrolase PHL7 bound to its product. Nat Commun 14, 1905 (2023). https://doi.org/10.1038/41467-023-37415-x; Erickson, E., Gado, J. E., Avilan, L. et al. Sourcing thermotolerant poly(ethylene terephthalate) hydrolase scaffolds from natural diversity. Nat Commun 13, 7850 (2022). https://doi.org/10.1038/41467-022-35237-x; Yoshida S, Hiraga K, Takchana T, Taniguchi I, Yamaji H, Macda Y, Toyohara K, Miyamoto K, Kimura Y, Oda K. A bacterium that degrades and assimilates poly(ethylene terephthalate). Science. 2016 Mar. 11; 351(6278):1196-9. doi: 10.1126/science.aad6359. PMID: 26965627; Han X, Liu W, Huang J W, Ma J, Zheng Y, Ko T P, Xu L, Cheng Y S, Chen C C, Guo R T. Structural insight into catalytic mechanism of PET hydrolase. Nat Commun. 2017 Dec. 13; 8(1):2106. doi: 10.1038/s41467-017-02255-z. PMID: 29235460; PMCID: PMC5727383; Cui H, Eltoukhy L, Zhang L, Markel U, Jaeger K E, Davari M D, Schwaneberg U. Less Unfavorable Salt Bridges on the Enzyme Surface Result in More Organic Cosolvent Resistance. Angew Chem Int Ed Engl. 2021 May 10; 60(20):11448-11456. doi: 10.1002/anic.202101642. Epub 2021 Apr. 7. PMID: 33687787; PMCID: PMC8252522; Son J, Kalafatovic D, Kumar M, Yoo B, Cornejo M A, Contel M, Ulijn R V. Customizing Morphology, Size, and Response Kinetics of Matrix Metalloproteinase-Responsive Nanostructures by Systematic Peptide Design. ACS Nano. 2019 Feb. 26; 13(2):1555-1562. doi: 10.1021/acsnano.8b07401. Epub 2019 Jan. 30. PMID: 30689363; PMCID: PMC6475088; Lu H, Diaz D J, Czarnecki N J, Zhu C, Kim W, Shroff R, Acosta D J, Alexander B R, Cole H O, Zhang Y, Lynd N A, Ellington A D, Alper H S. Machine learning-aided engineering of hydrolases for PET depolymerization. Nature. 2022 April; 604(7907):662-667. doi: 10.1038/s41586-022-04599-z. Epub 2022 Apr. 27. PMID: 35478237; Bell, E. L., Smithson, R., Kilbride, S. et al. Directed evolution of an efficient and thermostable PET depolymerase. Nat Catal 5, 673-681 (2022). https://doi.org/10.1038/941929-022-00821-3; Zhenkun Shi, Rui Deng, Qianqian Yuan, Zhitao Mao, Ruoyu Wang, Haoran Li, Xiaoping Liao, Hongwu Ma. Enzyme Commission Number Prediction and Benchmarking with Hierarchical Dual-core Multitask Learning Framework. Research. 2023; 6:0153.DOI:10.34133/research.0153; Danso D, Schmeisser C, Chow J, Zimmermann W, Wei R, Leggewie C, Li X, Hazen T, Streit W R. New Insights into the Function and Global Distribution of Polyethylene Terephthalate (PET)-Degrading Bacteria and Enzymes in Marine and Terrestrial Metagenomes. Appl Environ Microbiol. 2018 Apr. 2; 84(8):c02773-17. doi: 10.1128/AEM.02773-17. PMID: 29427431; PMCID: PMC5881046; Blázquez-Sánchez P, Engelberger F, Cifuentes-Anticevic J, Sonnendecker C, Griñón A, Reyes J, Dícz B, Guixć V, Richter P K, Zimmermann W, Ramírez-Sarmiento C A. Antarctic Polyester Hydrolases Degrade Aliphatic and Aromatic Polyesters at Moderate Temperatures. Appl Environ Microbiol. 2022 Jan. 11; 88(1):c0184221. doi: 10.1128/AEM.01842-21. Epub 2021 Oct. 27. PMID: 34705547; PMCID: PMC8752145; Chen X, Liu M, Zhang P, Leung S S Y, Xia J. Membrane-Permeable Antibacterial Enzyme against Multidrug-Resistant Acinctobacter baumannii. ACS Infect Dis. 2021 Aug. 13; 7(8):2192-2204. doi: 10.1021/acsinfecdis. 1c00222. Epub 2021 Jul. 7. PMID: 34232613; Avilan L, Lichtenstein B R, König G, Zahn M, Allen M D, Oliveira L, Clark M, Bemmer V, Graham R, Austin H P, Dominick G, Johnson C W, Beckham G T, McGechan J E, Pickford A R. Concentration-Dependent Inhibition of Mesophilic PETases on Poly(ethylene terephthalate) Can Be Eliminated by Enzyme Engineering. ChemSusChem. 2023 Apr. 21; 16(8):e202202277. doi: 10.1002/cssc.202202277. Epub 2023 Mar. 23. PMID: 36811288; Meyer Cifuentes I E, Wu P, Zhao Y, Liu W, Neumann-Schaal M, Pfaff L, Barys J, Li Z, Gao J, Han X, Bornscheuer U T, Wei R, Öztürk B. Molecular and Biochemical Differences of the Tandem and Cold-Adapted PET Hydrolases Ple628 and Ple629, Isolated From a Marine Microbial Consortium. Front Bioeng Biotechnol. 2022 Jul. 21; 10:930140. doi: 10.3389/fbioc.2022.930140. PMID: 35935485; PMCID: PMC9350882; Inglis G D, Yanke L J, Selinger L B. Cutinolytic esterase activity of bacteria isolated from mixed-plant compost and characterization of a cutinase gene from Pseudomonas pseudoalcaligenes. Can J Microbiol. 2011 November; 57(11):902-13. doi: 10.1139/w11-083. Epub 2011 Oct. 26. PMID: 22029433; Haernvall K, Zitzenbacher S, Wallig K, Yamamoto M, Schick M B, Ribitsch D, Guebitz G M. Hydrolysis of Ionic Phthalic Acid Based Polyesters by Wastewater Microorganisms and Their Enzymes. Environ Sci Technol. 2017 Apr. 18; 51(8):4596-4605. doi: 10.1021/acs.cst.7b00062. Epub 2017 Apr. 7. PMID: 28345898; Bollinger A, Thies S, Knieps-Grünhagen E, Gertzen C, Kobus S, Höppner A, Ferrer M, Gohlke H, Smits S H J, Jaeger K E. A Novel Polyester Hydrolase From the Marine Bacterium Pseudomonas acstusnigri-Structural and Functional Insights. Front Microbiol. 2020 Feb. 13; 11:114. doi: 10.3389/fmicb.2020.00114. PMID: 32117139; PMCID: PMC7031157; Macromolecules 2009, 42, 14, 5128-5138, Publication Date: Jul. 2, 2009, https://doi.org/10.1021/ma9005318; Wallace M, Green C R, Roberts L S, Lec Y M, McCarville J L, Sanchez-Gurmaches J, Meurs N, Gengatharan J M, Hover J D, Phillips S A, Ciaraldi T P, Guertin D A, Cabrales P, Ayres J S, Nomura D K, Loomba R, Metallo C M. Enzyme promiscuity drives branched-chain fatty acid synthesis in adipose tissues. Nat Chem Biol. 2018 November; 14(11): 1021-1031. doi: 10.1038/s41589-018-0132-2. Epub 2018 Oct. 16. PMID: 30327559; PMCID: PMC6245668; Eiamthong B, Meesawat P, Wongsatit T, Jitdec J, Sangsri R, Patchsung M, Aphicho K, Suraritdechachai S, Huguenin-Dezot N, Tang S, Suginta W, Paosawatyanyong B, Babu M M, Chin J W, Pakotiprapha D, Bhanthumnavin W, Uttamapinant C. Discovery and Genetic Code Expansion of a Polyethylene Terephthalate (PET) Hydrolase from the Human Saliva Metagenome for the Degradation and Bio-Functionalization of PET. Angew Chem Int Ed Engl. 2022 Sep. 12; 61(37):c202203061. doi: 10.1002/anic.202203061. Epub 2022 Jun. 21. PMID: 35656865; PMCID: PMC7613822; Sagong H Y, Son H F, Sco H, Hong H, Lee D, Kim K J. Implications for the PET decomposition mechanism through similarity and dissimilarity between PETases from Rhizobacter gummiphilus and Ideonella sakaiensis. J Hazard Mater. 2021 Aug. 15; 416:126075. doi: 10.1016/j.jhazmat.2021.126075. Epub 2021 May 11. PMID: 34492896; Erickson E, Gado J E, Avilán L, Bratti F, Brizendine R K, Cox P A, Gill R, Graham R, Kim D J, König G, Michener W E, Poudel S, Ramirez K J, Shakespeare T J, Zahn M, Boyd E S, Payne C M, DuBois J L, Pickford A R, Beckham G T, McGechan J E. Sourcing thermotolerant poly(ethylene terephthalate) hydrolase scaffolds from natural diversity. Nat Commun. 2022 Dec. 21; 13(1):7850. doi: 10.1038/s41467-022-35237-x. PMID: 36543766; PMCID: PMC9772341; Shirke A N, White C, Englaender J A, Zwarycz A, Butterfoss G L, Linhardt R J, Gross R A. Stabilizing Leaf and Branch Compost Cutinase (LCC) with Glycosylation: Mechanism and Effect on PET Hydrolysis. Biochemistry. 2018 Feb. 20; 57(7): 1190-1200. doi: 10.1021/acs.biochem.7b01189. Epub 2018 Jan. 30. PMID: 29328676; Xi X, Ni K, Hao H, Shang Y, Zhao B, Qian Z. Secretory expression in Bacillus subtilis and biochemical characterization of a highly thermostable polyethylene terephthalate hydrolase from bacterium HR29. Enzyme Microb Technol. 2021 February; 143:109715. doi: 10.1016/j.enzmictec.2020.109715. Epub 2020 Nov. 18. PMID: 33375975; Dresler K, van den Heuvel J, Müller R J, Deckwer W D. Production of a recombinant polyester-cleaving hydrolase from Thermobifida fusca in Escherichia coli. Bioprocess Biosyst Eng. 2006 August; 29(3): 169-83. doi: 10.1007/s00449-006-0069-9. Epub 2006 Jun. 13. PMID: 16770590; PMCID: PMC1705536; Müller, R. J., Schrader, H., Profe, J., Dresler, K., & Deckwer, W. D. (2005). Enzymatic Degradation of Poly (ethylene terephthalate): Rapid Hydrolyse using a Hydrolase from T. fusca. Macromolecular Rapid Communications, 26(17), 1400-1405. Kleeberg I, Welzel K, Vandenheuvel J, Müller R J, Deckwer W D. Characterization of a new extracellular hydrolase from Thermobifida fusca degrading aliphatic-aromatic copolyesters. Biomacromolecules. 2005 January-February; 6(1):262-70. doi: 10.1021/bm049582t. PMID: 15638529; Macromolecules 2011, 44, 12, 4632-4640, Publication Date: May 20, 2011,https://doi.org/10.1021/ma200949p; Furukawa M, Kawakami N, Tomizawa A, Miyamoto K. Efficient Degradation of Poly(ethylene terephthalate) with Thermobifida fusca Cutinase Exhibiting Improved Catalytic Activity Generated using Mutagenesis and Additive-based Approaches. Sci Rep. 2019 Nov. 5; 9(1):16038. doi: 10.1038/s41598-019-52379-z. PMID: 31690819; PMCID: PMC6831586; Roth C, Wei R, Oeser T, Then J, Föllner C, Zimmermann W, Sträter N. Structural and functional studies on a thermostable polyethylene terephthalate degrading hydrolase from Thermobifida fusca. Appl Microbiol Biotechnol. 2014 September; 98(18):7815-23. doi: 10.1007/s00253-014-5672-0. Epub 2014 Apr. 13. PMID: 24728714; Wei R, Oeser T, Schmidt J, Meier R, Barth M, Then J, Zimmermann W. Engineered bacterial polyester hydrolases efficiently degrade polyethylene terephthalate due to relieved product inhibition. Biotechnol Bioeng. 2016 August; 113(8): 1658-65. doi: 10.1002/bit.25941. Epub 2016 Feb. 4. PMID: 26804057; Ribitsch D, Hromic A, Zitzenbacher S, Zartl B, Gamerith C, Pellis A, Jungbauer A, Łyskowski A, Steinkellner G, Gruber K, Tscheliessnig R, Herrero Acero E, Guebitz G M. Small cause, large effect: Structural characterization of cutinases from Thermobifida cellulosilytica. Biotechnol Bioeng. 2017 November; 114(11):2481-2488. doi: 10.1002/bit.26372. Epub 2017 Aug. 15. PMID: 28671263; Ribitsch D, Herrero Acero E, Greimel K, Dellacher A, Zitzenbacher S, Marold A, Rodriguez R D, Steinkellner G, Gruber K, Schwab H, et al. A New Esterase from Thermobifida halotolerans Hydrolyses Polyethylene Terephthalate (PET) and Polylactic Acid (PLA). Polymers. 2012; 4(1):617-629. https://doi.org/10.3390/polym4010617; Kitadokoro K, Kakara M, Matsui S, Osokoshi R, Thumarat U, Kawai F, Kamitani S. Structural insights into the unique polylactate-degrading mechanism of Thermobifida alba cutinase. FEBS J. 2019 June; 286(11):2087-2098. doi: 10.1111/fcbs.14781. Epub 2019 Feb. 28. PMID: 30761732; Miyakawa T, Mizushima H, Ohtsuka J, Oda M, Kawai F, Tanokura M. Structural basis for the Ca(2+)-enhanced thermostability and activity of PET-degrading cutinase-like enzyme from Saccharomonospora viridis AHK190. Appl Microbiol Biotechnol. 2015 May; 99(10):4297-307. doi: 10.1007/s00253-014-6272-8. Epub 2014 Dec. 11. PMID: 25492421; Kawai F, Oda M, Tamashiro T, Waku T, Tanaka N, Yamamoto M, Mizushima H, Miyakawa T, Tanokura M. A novel Ca2-activated, thermostabilized polyesterase capable of hydrolyzing polyethylene terephthalate from Saccharomonospora viridis AHK190. Appl Microbiol Biotechnol. 2014 December; 98(24): 10053-64. doi: 10.1007/s00253-014-5860-y. Epub 2014 Jun. 15. PMID: 24929560; Oda M, Yamagami Y, Inaba S, Oida T, Yamamoto M, Kitajima S, Kawai F. Enzymatic hydrolysis of PET: functional roles of three Ca2+ ions bound to a cutinase-like enzyme, Cut190*, and its engineering for improved activity. Appl Microbiol Biotechnol. 2018 December; 102(23):10067-10077. doi: 10.1007/s00253-018-9374-x. Epub 2018 Sep. 24. PMID: 30250976; Jabloune R, Khalil M, Ben Moussa I E, Simao-Beaunoir A M, Lerat S, Brzezinski R, Beaulicu C. Enzymatic Degradation of p-Nitrophenyl Esters, Polyethylene Terephthalate, Cutin, and Suberin by Sub1, a Suberinase Encoded by the Plant Pathogen Streptomyces scabies. Microbes Environ. 2020; 35(1):ME19086. doi: 10.1264/jsme2.ME19086. PMID: 32101840; PMCID: PMC7104285; Carr C M, Keller M B, Paul B, Schubert S W, Clausen K S R, Jensen K, Clarke D J, Westh P, Dobson A D W. Purification and biochemical characterization of SM14est, a PET-hydrolyzing enzyme from the marine sponge-derived Streptomyces sp. SM14. Front Microbiol. 2023 May 12; 14:1170880. doi: 10.3389/fmicb.2023.1170880. PMID: 37250061; PMCID: PMC10213408; Tiong E, Koo Y S, Bi J, Koduru L, Koh W, Lim Y H, Wong F T. Expression and engineering of PET-degrading enzymes from Microbispora, Nonomuraea, and Micromonospora. Appl Environ Microbiol. 2023 Nov. 29; 89(11):e0063223. doi: 10.1128/aem.00632-23. Epub 2023 Nov. 9. PMID: 37943056; PMCID: PMC10686063; Ribitsch D, Heumann S, Trotscha E, Herrero Acero E, Greimel K, Leber R, Birner-Gruenberger R, Deller S, Eiteljoerg I, Remler P, Weber T, Siegert P, Maurer K H, Donelli I, Freddi G, Schwab H, Guebitz G M. Hydrolysis of polyethyleneterephthalate by p-nitrobenzylesterase from Bacillus subtilis. Biotechnol Prog. 2011 July; 27(4):951-60. doi: 10.1002/btpr.610. Epub 2011 May 13. PMID: 21574267; Perz V, Zumstein M T, Sander M, Zitzenbacher S, Ribitsch D, Guebitz G M. Biomimetic Approach to Enhance Enzymatic Hydrolysis of the Synthetic Polyester Poly(1,4-butylene adipate): Fusing Binding Modules to Esterases. Biomacromolecules. 2015 Dec. 14; 16(12):3889-96. doi: 10.1021/acs.biomac.5b01219. Epub 2015 Nov. 24. PMID: 26566664; Distaso M A, Chernikova T N, Bargicla R, Coscolín C, Stogios P, Gonzalez-Alfonso J L, Lemak S, Khusnutdinova A N, Plou F J, Evdokimova E, Savchenko A, Lunev E A, Yakimov M M, Golyshina O V, Ferrer M, Yakunin A F, Golyshin P N. Thermophilic Carboxylesterases from Hydrothermal Vents of the Volcanic Island of Ischia Active on Synthetic and Biobased Polymers and Mycotoxins. Appl Environ Microbiol. 2023 Feb. 28; 89(2):c0170422. doi: 10.1128/acm.01704-22. Epub 2023 Jan. 31. PMID: 36719236; PMCID: PMC9972953; Konstantinos Makryniotis, Efstratios Nikolaivits, Christina Gkountela, Stamatina Vouyiouka, Evangelos Topakas, Discovery of a polyesterase from Deinococcus maricopensis and comparison to the benchmark LCCICCG suggests high potential for semi-crystalline post-consumer PET degradation, Journal of Hazardous Materials, Volume 455, 2023, 131574, ISSN 0304-3894, https://doi.org/10.1016/j.jhazmat.2023.131574; ACS Catal. 2021, 11, 14, 8550-8564, Publication Date: Jun. 29, 2021, https://doi.org/10.1021/acscatal.1c01204; Carnicl, A., Valoni, É., Nicomedes, J., Gomes, A. D., & Castro, A. M. (2017). Lipase from Candida antarctica (CALB) and cutinase from Humicola insolens act synergistically for PET hydrolysis to terephthalic acid. Process Biochemistry, 59, 84-90; Brackmann R, de Oliveira Veloso C, de Castro A M, Langone M A P. Enzymatic post-consumer poly(ethylene terephthalate) (PET) depolymerization using commercial enzymes. 3 Biotech. 2023 May; 13(5):135. doi: 10.1007/s13205-023-03555-6. Epub 2023 Apr. 25. PMID: 37124991; PMCID: PMC10130296; Silva, C. M., Carneiro, F., O'Neill, A., Fonseca, L. P., Cabral, J. S., Guebitz, G., & Cavaco-Paulo, A. (2005). Cutinase—a new tool for biomodification of synthetic fibers. Journal of Polymer Science Part A: Polymer Chemistry, 43(11), 2448-2450; Dimarogona, M., Nikolaivits, E., Kanelli, M., Christakopoulos, P., Sandgren, M., & Topakas, E. (2015). Structural and functional studies of a Fusarium oxysporum cutinase with polyethylene terephthalate modification potential. Biochimica et Biophysica Acta (BBA)-General Subjects, 1850(11), 2308-2317; Brueckner, T., Eberl, A., Heumann, S., Rabe, M., & Guebitz, G. M. (2008). Enzymatic and chemical hydrolysis of poly (ethylene terephthalate) fabrics. Journal of Polymer Science Part A: Polymer Chemistry, 46(19), 6435-6443; Eberl, A., Heumann, S., Brückner, T., Araujo, R., Cavaco-Paulo, A., Kaufmann, F., . . . & Guebitz, G. M. (2009). Enzymatic surface hydrolysis of poly (ethylene terephthalate) and bis (benzoyloxyethyl) terephthalate by lipase and cutinase in the presence of surface active molecules. Journal of biotechnology, 143(3), 207-212; Vázquez-Alcántara, L., Oliart-Ros, R. M., García-Bórquez, A., & Peña-Montes, C. (2021). Expression of a cutinase of Moniliophthora roreri with polyester and PET-plastic residues degradation activity. Microbiology Spectrum, 9(3), e00976-21; Robles-Martín, A., Amigot-Sánchez, R., Fernandez-Lopez, L., Gonzalez-Alfonso, J. L., Roda, S., Alcolea-Rodriguez, V., . . . & Guallar, V. (2023). Sub-micro- and nano-sized polyethylene terephthalate deconstruction with engineered protein nanopores. Nature Catalysis, 1-12; Sevilla M E, Garcia M D, Perez-Castillo Y, Armijos-Jaramillo V, Casado S, Vizuete K, Debut A, Cerda-Mejía L. Degradation of PET Bottles by an Engineered Ideonella sakaiensis PETase. Polymers. 2023; 15(7): 1779. https://doi.org/10.3390/polym15071779; Edwards S, León-Zayas R, Ditter R, Laster H, Shechan G, Anderson O, Beattie T, Mellies J L. Microbial Consortia and Mixed Plastic Waste: Pangenomic Analysis Reveals Potential for Degradation of Multiple Plastic Types via Previously Identified PET Degrading Bacteria. International Journal of Molecular Sciences. 2022; 23(10):5612.https://doi.org/10.3390/jms23105612; Ho, N. H. E., Effendi, S. S. W., Ting, W. W., Yi, Y. C., Yu, J. Y., Chang, J. S., & Ng, I. S. (2023). Heterologous expression and characterization of Aquabacterium parvum lipase, a close relative of Ideonella sakaiensis PETase in Escherichia coli. Biochemical Engineering Journal, 197, 108985; Qi, X., Ji, M., Yin, C.-F., Zhou, N.-Y. & Liu, Y. (2023) Glacier as a source of novel polyethylene terephthalate hydrolases. Environmental Microbiology, 25(12), 2822-2833. Available from: https://doi.org/10.1111/1462-2920.16516; Chen S, Tong X, Woodard R W, Du G, Wu J, Chen J. Identification and characterization of bacterial cutinase. J Biol Chem. 2008 Sep. 19; 283(38):25854-62. doi: 10.1074/jbc.M800848200. Epub 2008 Jul. 24. PMID: 18658138; PMCID: PMC3258855; Su L, Woodard R W, Chen J, Wu J. Extracellular location of Thermobifida fusca cutinase expressed in Escherichia coli BL21(DE3) without mediation of a signal peptide. Appl Environ Microbiol. 2013 July; 79(14):4192-8. doi: 10.1128/AEM.00239-13. Epub 2013 Apr. 19. PMID: 23603671; PMCID: PMC3697513; Lykidis A, Mavromatis K, Ivanova N, Anderson I, Land M, DiBartolo G, Martinez M, Lapidus A, Lucas S, Copeland A, Richardson P, Wilson D B, Kyrpides N. Genome sequence and analysis of the soil cellulolytic actinomycete Thermobifida fusca Y X. J Bacteriol. 2007 March; 189(6):2477-86. doi: 10.1128/J B.01899-06. Epub 2007 Jan. 5. PMID: 17209016; PMCID: PMC1899369; Hegde K, Vecranki V D. Production optimization and characterization of recombinant cutinases from Thermobifida fusca sp. NRRL B-8184. Appl Biochem Biotechnol. 2013 June; 170(3):654-75. doi: 10.1007/s12010-013-0219-x. Epub 2013 Apr. 19. PMID: 23604968; Wei R, Oeser T, Then J, Kühn N, Barth M, Schmidt J, Zimmermann W. Functional characterization and structural modeling of synthetic polyester-degrading hydrolases from Thermomonospora curvata. AMB Express. 2014 Jun. 3; 4:44. doi: 10.1186/s13568-014-0044-9. PMID: 25405080; PMCID: PMC4231364; Hu X, Thumarat U, Zhang X, Tang M, Kawai F. Diversity of polyester-degrading bacteria in compost and molecular analysis of a thermoactive esterase from Thermobifida alba AHK119. Appl Microbiol Biotechnol. 2010 June; 87(2):771-9. doi: 10.1007/s00253-010-2555-x. PMID: 20393707; Almeida E L, Carrillo Rincón A F, Jackson S A, Dobson A D W. In silico Screening and Heterologous Expression of a Polyethylene Terephthalate Hydrolase (PETase)-Like Enzyme (SM14est) With Polycaprolactone (PCL)-Degrading Activity, From the Marine Sponge-Derived Strain Streptomyces sp. SM14. Front Microbiol. 2019 Oct. 1; 10:2187. doi: 10.3389/fmich.2019.02187. PMID: 31632361; PMCID: PMC6779837; Zhang, H., Dierkes, R. F., Perez-Garcia, P., Costanzi, E., Dittrich, J., Cea, P. A., Gurschke, M., Applegate, V., Partus, K., Schmeisser, C., Pfleger, C., Gohlke, H., Smits, S. H. J., Chow, J. and Streit, W. R. (2024), The metagenome-derived esterase PET40 is highly promiscuous and hydrolyses polyethylene terephthalate (PET). FEBS J, 291: 70-91. https://doi.org/10.1111/febs.16924; Zhang H, Perez-Garcia P, Dierkes R F, Applegate V, Schumacher J, Chibani C M, Sternagel S, Preuss L, Weigert S, Schmeisser C, Danso D, Pleiss J, Almeida A, Höcker B, Hallam S J, Schmitz R A, Smits S H J, Chow J, Streit W R. The Bacteroidetes Aequorivita sp. and Kaistella jeonii Produce Promiscuous Esterases With PET-Hydrolyzing Activity. Front Microbiol. 2022 Jan. 5; 12:803896. doi: 10.3389/fmicb.2021.803896. PMID: 35069509; PMCID: PMC8767016; and Perez-Garcia, P., Chow, J., Costanzi, E. et al. An archaeal lid-containing feruloyl esterase degrades polyethylene terephthalate. Commun Chem 6, 193 (2023). https://doi.org/10.1038/s42004-023-00998-z.

Further examples of such polymer-degrading enzymes that are useful in methods and compositions provided herein are listed Table 1. In some embodiments, a polymer-degrading enzyme useful in methods and compositions provided herein has an amino acid sequence set forth in any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38. In some embodiments, the polymer-degrading enzyme is a variant of any one of the foregoing enzymes in which the variant has an insertion, deletion, or substitution of up to 1, 2, 3, 4, 5, 6, 7, 8, 9, 10, 11, 12, 13, 14, 15, 16, 17, 18, 19, or 20 amino acids compared with an amino acid sequence set forth in any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38. In some embodiments, the polymer-degrading enzyme is a variant of any one of the foregoing enzymes, in which the variant has at least 85%, 86%, 87%, 88%, 89%, 90%, 91%, 92%, 93%, 94%, 95%, 96%, 97%, 98%, or 99% identity compared to an amino acid sequence set forth in any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38.

TABLE 1
Examples of Polymer-degrading enzymes

| Accession | | | | | | | |
|---|---|---|---|---|---|---|---|
| Number | | | | | | | |
| (UniProt or | SEQ ID | | Common Name | | | EC | ESTHER |
| GenBank) | NO: | Sequence | (if any) | Pfam | InterPro | number | family |
| A0A075B5G4 | 1 | QLGAIENGLES | HiC | PF01083; | IPR029058; | 3.1.1.74 | Cutinase |
| | | GSANACPDAIL | | | IPR000675; | | |
| | | IFARGSTEPGN | | | IPR043580; | | |
| | | MGITVGPALA | | | IPR043579; | | |
| | | NGLESHIRNIW | | | IPR011150; | | |
| | | IQGVGGPYDA | | | | | |
| | | ALATNFLPRG | | | | | |
| | | TSQANIDEGK | | | | | |
| | | RLFALANQKC | | | | | |
| | | PNTPVVAGGY | | | | | |
| | | SQGAALIAAA | | | | | |
| | | VSELSGAVKE | | | | | |
| | | QVKGVALFGY | | | | | |
| | | TQNLQNRGGI | | | | | |
| | | PNYPRERTKV | | | | | |
| | | FCNVGDAVCT | | | | | |
| | | GTLIITPAHLS | | | | | |
| | | YTIEARGEAA | | | | | |
| | | RFLRDRIRA | | | | | |
| A0A0G3BI90 | 2 | MPPDCVLPRR | PET12; PbPL | PF01738; | IPR029058; | 3.1.1.101 | Polyesterase- |
| | | LAAAALLASA | | | IPR002925; | | lipase- |
| | | TLVPLSAAAQ | | | | | cutinase |
| | | TNPYQRGPDP | | | | | |
| | | TTRDLEDSRGP | | | | | |
| | | FRYASTNVRSP | | | | | |
| | | SGYGAGTIYY | | | | | |
| | | PTDVSGSVGA | | | | | |
| | | VAVVPGYLAR | | | | | |
| | | QSSIRWWGPR | | | | | |
| | | LASHGFVVITL | | | | | |
| | | DTRSTSDQPAS | | | | | |
| | | RSAQQMAALR | | | | | |
| | | QVVALSETRSS | | | | | |
| | | PIYGKVDPNRL | | | | | |
| | | AVMGWSMGG | | | | | |
| | | GGTLISARDNP | | | | | |
| | | SLKAAVPFAP | | | | | |
| | | WHNTANFSGV | | | | | |
| | | QVPTLVIACEN | | | | | |
| | | DTVAPISRHAS | | | | | |
| | | SFYNSFSSSLA | | | | | |
| | | KAYLEINNGS | | | | | |
| | | HTCANTGNSN | | | | | |
| | | QALIGKYGVA | | | | | |
| | | WIKRFVDNDT | | | | | |
| | | RYSPFLCGAPH | | | | | |
| | | QADLRSSRLSE | | | | | |
| | | YRESCPY | | | | | |
| A0A0K8P6T7 | 3 | MNFPRASRLM | isPETase | PF01738; | IPR029058; | | Polyesterase- |
| | | QAAVLGGLM | | | IPR002925; | | lipase- |
| | | AVSAAATAQT | | | | | cutinase |
| | | NPYARGPNPT | | | | | |
| | | AASLEASAGPF | | | | | |
| | | TVRSFTVSRPS | | | | | |
| | | GYGAGTVYYP | | | | | |
| | | TNAGGTVGAI | | | | | |
| | | AIVPGYTARQS | | | | | |
| | | SIKWWGPRLA | | | | | |
| | | SHGFVVITIDT | | | | | |
| | | NSTLDQPSSRS | | | | | |
| | | SQQMAALRQV | | | | | |
| | | ASLNGTSSSPI | | | | | |
| | | YGKVDTARM | | | | | |
| | | GVMGWSMGG | | | | | |
| | | GGSLISAANNP | | | | | |
| | | SLKAAAPQAP | | | | | |
| | | WDSSTNFSSV | | | | | |
| | | TVPTLIFACEN | | | | | |
| | | DSIAPVNSSAL | | | | | |
| | | PIYDSMSRNA | | | | | |
| | | KQFLEINGGSH | | | | | |
| | | SCANSGNSNQ | | | | | |
| | | ALIGKKGVAW | | | | | |
| | | MKRFMDNDT | | | | | |
| | | RYSTFACENP | | | | | |
| | | NSTRVSDFRT | | | | | |
| | | ANCS | | | | | |
| A0A1F4JXW8 | 4 | MAVGSMLLS | BurPL | PF01738; | IPR029058; | | Polyesterase- |
| | | MAAQAQVVV | | | IPR002925; | | lipase- |
| | | FEETFSTGLGK | | | | | cutinase |
| | | FTAAGSVVTSS | | | | | |
| | | GAARLDGCYG | | | | | |
| | | CTDGSITSTAIS | | | | | |
| | | TVDFTGLRLSF | | | | | |
| | | DRVTSGLDSG | | | | | |
| | | EAGIAEFSTNG | | | | | |
| | | STYTAVESIRT | | | | | |
| | | ASGRVTFNLPT | | | | | |
| | | SAENQSGLRL | | | | | |
| | | RFRINASLSSE | | | | | |
| | | TYTVDNIRLEG | | | | | |
| | | TSGSGGGTTN | | | | | |
| | | PFEKGPDPTKT | | | | | |
| | | MLEASTGPFT | | | | | |
| | | YTTTTVSSTTA | | | | | |
| | | SGYRQGTIYHP | | | | | |
| | | TNVTGPFAAV | | | | | |
| | | AVVPGYLASQ | | | | | |
| | | SSINWWGPRL | | | | | |
| | | ASHGFVVITID | | | | | |
| | | TNSTSDQPPSR | | | | | |
| | | ATQLMAALNQ | | | | | |
| | | LKTFSNTSSHP | | | | | |
| | | IYRKVDPNRL | | | | | |
| | | GVMGWSMGG | | | | | |
| | | GGTLIAARDN | | | | | |
| | | PTLKAAIPFAP | | | | | |
| | | WNSSTNFSTV | | | | | |
| | | SVPTLIIACESD | | | | | |
| | | STAPVNSHASP | | | | | |
| | | FYNSLPSTTKK | | | | | |
| | | AYLEMNNGSH | | | | | |
| | | SCANSGNSNA | | | | | |
| | | GLIGKYGVSW | | | | | |
| | | MKRFMDNDT | | | | | |
| | | RFSPYLCGAPH | | | | | |
| | | QADLSLTAIDE | | | | | |
| | | YRENCPY | | | | | |
| A0A1H6AD45 | 5 | MPFNKKSVLA | PE-H | PF01738; | IPR029058; | | Polyesterase- |
| | | LCGAGALLFS | | | IPR002925; | | lipase- |
| | | MSALANNPAP | | | | | cutinase |
| | | TDPGDSGGGS | | | | | |
| | | AYQRGPDPSV | | | | | |
| | | SFLEADRGQY | | | | | |
| | | SVRSSRVSSLV | | | | | |
| | | SGFGGGTIYYP | | | | | |
| | | TGTTGTMGAV | | | | | |
| | | VVIPGFVSAES | | | | | |
| | | SIDWWGPKLA | | | | | |
| | | SYGFVVMTID | | | | | |
| | | TNTGFDQPPSR | | | | | |
| | | ARQINNALDY | | | | | |
| | | LVSQNSRSSSP | | | | | |
| | | VRGMIDTNRL | | | | | |
| | | GVIGWSMGGG | | | | | |
| | | GTLRVASEGRI | | | | | |
| | | KAAIPLAPWD | | | | | |
| | | TTSYYASRSQ | | | | | |
| | | APTLIFACESD | | | | | |
| | | VIAPVLQHASP | | | | | |
| | | FYNSLPSSIDK | | | | | |
| | | AFVEINGGSH | | | | | |
| | | YCGNGGSIYN | | | | | |
| | | DVLSRFGVSW | | | | | |
| | | MKLHLDEDSR | | | | | |
| | | YKQFLCGPNH | | | | | |
| | | TSDSQISDYRG | | | | | |
| | | NCPY | | | | | |
| A0A1W6L588 | 6 | MFGKLPFARA | RgPETase | PF01738; | IPR029058; | | Polyesterase- |
| | | SLAVGALLLS | | | IPR002925; | | lipase- |
| | | AAAVAQTNPY | | | | | cutinase |
| | | QRGPDPTVSSL | | | | | |
| | | EATRGPFSTSS | | | | | |
| | | FTVSRPSGYG | | | | | |
| | | AGTVYYPTNA | | | | | |
| | | GGKVGAIAVV | | | | | |
| | | PGYTARQSSIN | | | | | |
| | | WWGPRLASH | | | | | |
| | | GFVVITIDTNS | | | | | |
| | | TLDQPSSRSSQ | | | | | |
| | | QMAALRQVVS | | | | | |
| | | LAGTSSSPIYN | | | | | |
| | | KVDTARLGV | | | | | |
| | | MGWSMGGGG | | | | | |
| | | SLISAKNNPSL | | | | | |
| | | RAAAPQAPWA | | | | | |
| | | QESFSSVTVPT | | | | | |
| | | LIVSCENDSIA | | | | | |
| | | PNSSHSFPFYN | | | | | |
| | | QMTRNKKAN | | | | | |
| | | LVINGGSHSCA | | | | | |
| | | NSGNSDAGLI | | | | | |
| | | GKYGVAWMK | | | | | |
| | | RFMDDDTRYS | | | | | |
| | | KFLCGAEHQA | | | | | |
| | | DLSKRAVEAY | | | | | |
| | | KENCPY | | | | | |
| A0A2H5Z9R5 | 7 | MQVVLGRVRS | BhrPETase | PF12695; | IPR029058; | 3.1.1.101 | Polyesterase- |
| | | AGLLAALLAL | | | IPR029059; | | lipase- |
| | | AAWALVWAS | | | | | cutinase |
| | | PSAEAQSNPY | | | | | |
| | | QRGPNPTRSA | | | | | |
| | | LTTDGPFSVAT | | | | | |
| | | YSVSRLSVSGF | | | | | |
| | | GGGVIYYPTG | | | | | |
| | | TTLTFGGIAMS | | | | | |
| | | PGYTADASSL | | | | | |
| | | AWLGRRLASH | | | | | |
| | | GFVVIVINTNS | | | | | |
| | | RLDFPDSRASQ | | | | | |
| | | LSAALNYLRT | | | | | |
| | | SSPSAVRARLD | | | | | |
| | | ANRLAVAGHS | | | | | |
| | | MGGGATLRIS | | | | | |
| | | EQIPTLKAGVP | | | | | |
| | | LTPWHTDKTF | | | | | |
| | | NTPVPQLIVGA | | | | | |
| | | EADTVAPVSQ | | | | | |
| | | HAIPFYQNLPS | | | | | |
| | | TTPKVYVELD | | | | | |
| | | NATHFAPNSP | | | | | |
| | | NAAISVYTISW | | | | | |
| | | MKLWVDNDT | | | | | |
| | | RYRQFLCNVN | | | | | |
| | | DPALSDFRSN | | | | | |
| | | NRHCQ | | | | | |
| A0A5P3LVN0 | 8 | MAACTDIDVV | Sub1 | PF01083; | IPR029058; | | Cutinase |
| | | SARGTFEPGTL | | | IPR000675; | | |
| | | GFIVGDPVYA | | | | | |
| | | ALQKKVAGKS | | | | | |
| | | LSSYKVNYPA | | | | | |
| | | DLSPTSAAQG | | | | | |
| | | NADLVNHVRS | | | | | |
| | | QAASCPNQRF | | | | | |
| | | VLVGYSQGAN | | | | | |
| | | VVDNSIGISSA | | | | | |
| | | GAVVGSPIVA | | | | | |
| | | TLPAALEPRVS | | | | | |
| | | AVLLFGNPIRA | | | | | |
| | | IGKSVTGTYQS | | | | | |
| | | RTIDFCAAGDP | | | | | |
| | | VCENGGGDVG | | | | | |
| | | AHLGYRANAD | | | | | |
| | | AAAAFAATKI | | | | | |
| A5I055 | 9 | MKKNLNKIAT | del71Cbotu_ | | IPR029058; | | Bacterial_ |
| | | IILLVFSMTLT | EstA | | | | lip_Fam1.5 |
| | | NFSMIVRAAEP | | | | | |
| | | KAQGTQKVES | | | | | |
| | | STTKKEVKDA | | | | | |
| | | EETIKIPTLEDI | | | | | |
| | | DNLIDSAEEVK | | | | | |
| | | SEEDINKMPPL | | | | | |
| | | KFPVEFPEVNT | | | | | |
| | | RSIIGGNNYPI | | | | | |
| | | VLVHGFMGFG | | | | | |
| | | RDELLGYKYW | | | | | |
| | | GGVVDLQEKL | | | | | |
| | | NASGHETYTA | | | | | |
| | | TVGPVSSNWD | | | | | |
| | | RACELYAYIV | | | | | |
| | | GGTVDYGEAH | | | | | |
| | | AKKFKHNRYG | | | | | |
| | | RTYPGIYKNIS | | | | | |
| | | NENKIHLIGHS | | | | | |
| | | MGGQTIRTLT | | | | | |
| | | QLLSEGSEEEI | | | | | |
| | | NCGQENISPLF | | | | | |
| | | EGGKHWIHSV | | | | | |
| | | STISTPNDGTT | | | | | |
| | | LSDLMPAKDLI | | | | | |
| | | SYTFGVLGTIT | | | | | |
| | | GKNKLFSSIYD | | | | | |
| | | LKLDQWGLK | | | | | |
| | | KQNGESQRDY | | | | | |
| | | IERVLDSNIWN | | | | | |
| | | STKDIATYDLS | | | | | |
| | | TEGAQELNTW | | | | | |
| | | VKAQPDVYYF | | | | | |
| | | SWTTQATKESI | | | | | |
| | | LTGHSVAQIGP | | | | | |
| | | MNPIFYPTANL | | | | | |
| | | MGRYSRNQK | | | | | |
| | | DLPIIDKKWFP | | | | | |
| | | NDGVVNCISQ | | | | | |
| | | DGPKLGSNDV | | | | | |
| | | IEQYNGGVKIG | | | | | |
| | | QWNAMPRIIN | | | | | |
| | | TDHMDIVGTF | | | | | |
| | | GNVKDWYMD | | | | | |
| | | YASFLSNLSR | | | | | |
| C3RYL0 | 10 | MPITARNTLAS | PET2 | PF01738; | IPR029058; | | Polyesterase- |
| | | LLLASSALLLS | | | IPR002925; | | lipase- |
| | | GTAFAANPPG | | | | | cutinase |
| | | GDPDPGCQTD | | | | | |
| | | CNYQRGPDPT | | | | | |
| | | DAYLEAASGP | | | | | |
| | | YTVSTIRVSSL | | | | | |
| | | VPGFGGGTIH | | | | | |
| | | YPTNAGGGK | | | | | |
| | | MAGIVVIPGYL | | | | | |
| | | SFESSIEWWGP | | | | | |
| | | RLASHGFVVM | | | | | |
| | | TIDTNTIYDQP | | | | | |
| | | SQRRDQIEAAL | | | | | |
| | | QYLVNQSNSS | | | | | |
| | | SSPISGMVDSS | | | | | |
| | | RLAAVGWSM | | | | | |
| | | GGGGTLQLAA | | | | | |
| | | DGGIKAAIALA | | | | | |
| | | PWNSSINDFNR | | | | | |
| | | IQVPTLIFACQ | | | | | |
| | | LDAIAPVALH | | | | | |
| | | ASPFYNRIPNT | | | | | |
| | | TPKAFFEMTG | | | | | |
| | | GDHWCANGG | | | | | |
| | | NIYSALLGKY | | | | | |
| | | GVSWMKLHL | | | | | |
| | | DQDTRYAPFL | | | | | |
| | | CGPNHAAQTL | | | | | |
| | | ISEYRGNCPY | | | | | |
| D1A2H1 | 11 | MKRTLKRALS | Teur0390 | PF03403; | IPR029058; | 3.1.1.3 | Polyesterase- |
| | | LLPAAALAAS | | | | | lipase- |
| | | ALVAASPAQA | | | | | cutinase |
| | | AANPYQRGPN | | | | | |
| | | PTEASITAARG | | | | | |
| | | PFNTAEITVSR | | | | | |
| | | LSVSGFGGGKI | | | | | |
| | | YYPTTTSEGTF | | | | | |
| | | GAIAISPGFTA | | | | | |
| | | YWSSLEWLGH | | | | | |
| | | RLASQGFVVIG | | | | | |
| | | IETNTTLDQPD | | | | | |
| | | QRGQQLLAAL | | | | | |
| | | DYLTQRSAVR | | | | | |
| | | DRVDASRLAV | | | | | |
| | | AGHSMGGGGS | | | | | |
| | | LEAAKARTSL | | | | | |
| | | KAAIPLAPWN | | | | | |
| | | LDKTWPEVRT | | | | | |
| | | PTLIIGGELDA | | | | | |
| | | VAPVATHSIPF | | | | | |
| | | YNSLSNAPEK | | | | | |
| | | AYLELDNASH | | | | | |
| | | FFPNITNTQMA | | | | | |
| | | KYMIAWMKR | | | | | |
| | | FIDDDTRYTQF | | | | | |
| | | LCPPPSTGLLS | | | | | |
| | | DFSDARFTCP | | | | | |
| | | M | | | | | |
| D1A9G5 | 12 | MSLRKSFGLLS | Tour1278 | PF03403; | IPR029058; | 3.1.1.3 | Polyesterase- |
| | | ATAALVAGLV | | | | | lipase- |
| | | AAPPAQAAAN | | | | | cutinase |
| | | PYQRGPDPTES | | | | | |
| | | LLRAARGPFA | | | | | |
| | | VSEQSVSRLSV | | | | | |
| | | SGFGGGRIYYP | | | | | |
| | | TTTSQGTFGAI | | | | | |
| | | AISPGFTASWS | | | | | |
| | | SLAWLGPRLA | | | | | |
| | | SHGFVVIGIET | | | | | |
| | | NTRLDQPDSR | | | | | |
| | | GRQLLAALDY | | | | | |
| | | LTQRSSVRNR | | | | | |
| | | VDASRLAVAG | | | | | |
| | | HSMGGGGTLE | | | | | |
| | | AAKSRTSLKA | | | | | |
| | | AIPLAPWNLDK | | | | | |
| | | TWPEVRTPTLI | | | | | |
| | | IGGELDSIAPV | | | | | |
| | | ATHSIPFYNSL | | | | | |
| | | TNAREKAYLE | | | | | |
| | | LNNASHFFPQF | | | | | |
| | | SNDTMAKFMI | | | | | |
| | | SWMKRFIDDD | | | | | |
| | | TRYDQFLCPPP | | | | | |
| | | RAIGDISDYRD | | | | | |
| | | TCPHT | | | | | |
| D4Q9N1 | 13 | MSVTTPRREA | Est1 | PF12740; | IPR029058; | 3.1.1.101; | Polyesterase- |
| | | SLLSRAVAVA | | | IPR041127; | 3.1.1.74 | lipase- |
| | | AAAAATVALA | | | | | cutinase |
| | | APAQAANPYE | | | | | |
| | | RGPNPTESML | | | | | |
| | | EARSGPFSVSE | | | | | |
| | | ERASRLGADG | | | | | |
| | | FGGGTIYYPRE | | | | | |
| | | NNTYGAIAISP | | | | | |
| | | GYTGTQSSIA | | | | | |
| | | WLGERIASHG | | | | | |
| | | FVVIAIDTNTT | | | | | |
| | | LDQPDSRARQ | | | | | |
| | | LNAALDYMLT | | | | | |
| | | DASSSVRNRID | | | | | |
| | | ASRLAVMGHS | | | | | |
| | | MGGGGTLRLA | | | | | |
| | | SQRPDLKAAIP | | | | | |
| | | LTPWHLNKSW | | | | | |
| | | RDITVPTLIIGA | | | | | |
| | | DLDTIAPVSSH | | | | | |
| | | SEPFYNSIPSST | | | | | |
| | | DKAYLELNNA | | | | | |
| | | THFAPNITNKT | | | | | |
| | | IGMYSVAWLK | | | | | |
| | | RFVDEDTRYT | | | | | |
| | | QFLCPGPRTGL | | | | | |
| | | LSDVDEYRST | | | | | |
| | | CPF | | | | | |
| D7R6G8 | 14 | MTHQIVTTQY | BsEstB | PF00135; | IPR029058; | 3.1.1.- | Carb_B_ |
| | | GKVKGTTENG | | | IPR002018; | | Bacteria |
| | | VHKWKGIPYA | | | IPR019826; | | |
| | | KPPVGQWRFK | | | IPR019819; | | |
| | | APEPPEVWED | | | IPR000997; | | |
| | | VLDATAYGPI | | | | | |
| | | CPQPSDLLSLS | | | | | |
| | | YTELPRQSEDC | | | | | |
| | | LYVNVFAPDT | | | | | |
| | | PSQNLPVMVW | | | | | |
| | | IHGGAFYLGA | | | | | |
| | | GSEPLYDGSK | | | | | |
| | | LAAQGEVIVV | | | | | |
| | | TLNYRLGPFGF | | | | | |
| | | LHLSSFNEAYS | | | | | |
| | | DNLGLLDQAA | | | | | |
| | | ALKWVRENIS | | | | | |
| | | AFGGDPDNVT | | | | | |
| | | VFGESAGGMS | | | | | |
| | | LAALLAMPAA | | | | | |
| | | KGLFQKAIME | | | | | |
| | | SGASRTMTKE | | | | | |
| | | QAASTSAAFL | | | | | |
| | | QVLGINEGQL | | | | | |
| | | DKLHTVSAED | | | | | |
| | | SLKAADQLRI | | | | | |
| | | AEKENIFQLFF | | | | | |
| | | QPALDPKTLPE | | | | | |
| | | EPEKALAEGAA | | | | | |
| | | SGIPLLIGTTRD | | | | | |
| | | EGYLFFTPDSD | | | | | |
| | | VHSQETLDAA | | | | | |
| | | LEYLLGKPLA | | | | | |
| | | EKAADLYPRS | | | | | |
| | | LESQIHMMTD | | | | | |
| | | LLFWRPAVAY | | | | | |
| | | ASAQSHYAPV | | | | | |
| | | WMYRFDWHP | | | | | |
| | | KKPPYNKAFH | | | | | |
| | | ALELPFVFGNL | | | | | |
| | | DGLERMAKAE | | | | | |
| | | ITDEVKQLSHT | | | | | |
| | | IQSAWITFAKT | | | | | |
| | | GNPSTEAVNW | | | | | |
| | | PAYHEETRETL | | | | | |
| | | ILDSEITIENDP | | | | | |
| | | ESEKRQKLFPS | | | | | |
| | | KGE | | | | | |
| E9L VH7 | 15 | MANPYERGPN | Tha_Cut1 | PF12740; | IPR029058; | 3.1.1.101; | Polyesterase- |
| | | PTDALLEASSG | | | IPR041127; | 3.1.1.74 | lipase- |
| | | PFSVSEENVSR | | | | | cutinase |
| | | LSASGFGGGTI | | | | | |
| | | YYPRENNTYG | | | | | |
| | | AVAISPGYTGT | | | | | |
| | | EASIAWLGGRI | | | | | |
| | | ASHGFVVITID | | | | | |
| | | TITTLDQPDSR | | | | | |
| | | AEQLNAALNH | | | | | |
| | | MINRASSTVRS | | | | | |
| | | RIDSSRLAVM | | | | | |
| | | GHSMGGGGTP | | | | | |
| | | RLASQRPDLK | | | | | |
| | | AAIPLTPWHL | | | | | |
| | | NKNRSSVTVP | | | | | |
| | | TLIIGADLDTIA | | | | | |
| | | PVATHAKPFY | | | | | |
| | | NSLPSSISKAY | | | | | |
| | | LELDGATHFA | | | | | |
| | | PNIPNKIIGKYS | | | | | |
| | | VAWLKRFVD | | | | | |
| | | NDTRYTQFLC | | | | | |
| | | PGPRDGLFGE | | | | | |
| | | VEEYCSTCPF | | | | | |
| E9LVH8 | 16 | MANPYERGPN | The_Cut1 | PF12740; | IPR029058; | 3.1.1.101; | Polyesterase- |
| | | PTDALLEASSG | | | IPR041127; | 3.1.1.74 | lipase- |
| | | PFSVSEENVSR | | | | | cutinase |
| | | LSASGFGGGTI | | | | | |
| | | YYPRENNTYG | | | | | |
| | | AVAISPGYTGT | | | | | |
| | | EASIAWLGERI | | | | | |
| | | ASHGFVVITID | | | | | |
| | | TITTLDQPDSR | | | | | |
| | | AEQLNAALNH | | | | | |
| | | MINRASSTVRS | | | | | |
| | | RIDSSRLAVM | | | | | |
| | | GHSMGGGGTL | | | | | |
| | | RLASQRPDLK | | | | | |
| | | AAIPLTPWHL | | | | | |
| | | NKNWSSVTVP | | | | | |
| | | TLIIGADLDTIA | | | | | |
| | | PVATHAKPFY | | | | | |
| | | NSLPSSISKAY | | | | | |
| | | LELDGATHFA | | | | | |
| | | PNIPNKIIGKYS | | | | | |
| | | VAWLKRFVD | | | | | |
| | | NDTRYTQFLC | | | | | |
| | | PGPRDGLFGE | | | | | |
| | | VEEYRSTCPF | | | | | |
| E9LVH9 | 17 | MANPYERGPN | Thc_Cut2 | PF12740; | IPR029058; | 3.1.1.101; | Polyesterase- |
| | | PTDALLEARS | | | IPR041127; | 3.1.1.74 | lipase- |
| | | GPFSVSEERAS | | | | | cutinase |
| | | RFGADGFGGG | | | | | |
| | | TIYYPRENNTY | | | | | |
| | | GAVAISPGYT | | | | | |
| | | GTQASVAWLG | | | | | |
| | | ERIASHGFVVI | | | | | |
| | | TIDTNTTLDQP | | | | | |
| | | DSRARQLNAA | | | | | |
| | | LDYMINDASS | | | | | |
| | | AVRSRIDSSRL | | | | | |
| | | AVMGHSMGG | | | | | |
| | | GGTLRLASQR | | | | | |
| | | PDLKAAIPLTP | | | | | |
| | | WHLNKNWSS | | | | | |
| | | VRVPTLIIGAD | | | | | |
| | | LDTIAPVLTHA | | | | | |
| | | RPFYNSLPTSIS | | | | | |
| | | KAYLELDGAT | | | | | |
| | | HFAPNIPNKIIG | | | | | |
| | | KYSVAWLKRF | | | | | |
| | | VDNDTRYTQF | | | | | |
| | | LCPGPRDGLF | | | | | |
| | | GEVEEYRSTCP | | | | | |
| | | F | | | | | |
| F7IX06 | 18 | MSVTTPRRETS | Est119/ | PF12740; | IPR029058; | 3.1.1.101; | Polyesterase- |
| | | LLSRALRATA | Ta_Cut | | IPR041127; | 3.1.1.74 | lipase- |
| | | AAATAVVATV | | | | | cutinase |
| | | ALAAPAQAAN | | | | | |
| | | PYERGPNPTES | | | | | |
| | | MLEARSGPFS | | | | | |
| | | VSEERASRFG | | | | | |
| | | ADGFGGGTIY | | | | | |
| | | YPRENNTYGA | | | | | |
| | | IAISPGYTGTQ | | | | | |
| | | SSIAWLGERIA | | | | | |
| | | SHGFVVIAIDT | | | | | |
| | | NTTLDQPDSR | | | | | |
| | | ARQLNAALDY | | | | | |
| | | MLTDASSAVR | | | | | |
| | | NRIDASRLAV | | | | | |
| | | MGHSMGGGG | | | | | |
| | | TLRLASQRPDL | | | | | |
| | | KAAIPLTPWH | | | | | |
| | | LNKSWRDITV | | | | | |
| | | PTLIIGAEYDTI | | | | | |
| | | ASVTLHSKPFY | | | | | |
| | | NSIPSPTDKAY | | | | | |
| | | LELDGASHFA | | | | | |
| | | PNITNKTIGMY | | | | | |
| | | SVAWLKRFVD | | | | | |
| | | EDTRYTQFLCP | | | | | |
| | | GPRTGLLSDV | | | | | |
| | | EEYRSTCPF | | | | | |
| G2RAE6 | 19 | MKFLPILCAA | TtcutA | PF01083; | IPR029058; | 3.1.1.74 | Cutinase |
| | | GLAAAAPTQP | | | IPR000675; | | |
| | | AGEAAVEARQ | | | IPR043579; | | |
| | | LESDTANDLE | | | IPR011150; | | |
| | | NGVSSNCPKVI | | | | | |
| | | FICARGSTETG | | | | | |
| | | NLGSSVCPEV | | | | | |
| | | ANGLKNYYPN | | | | | |
| | | QLWVQGVGG | | | | | |
| | | AYTADLASNA | | | | | |
| | | LPGGTSTAAM | | | | | |
| | | QEAANMFNLA | | | | | |
| | | QQKCPNASVA | | | | | |
| | | AGGYSQGTAV | | | | | |
| | | VAGGIQSLSA | | | | | |
| | | AAKDQIKGVV | | | | | |
| | | LFGYTQAQQN | | | | | |
| | | HDTIPNFPVDK | | | | | |
| | | TMIFCAQGDL | | | | | |
| | | VCNGTLIVTA | | | | | |
| | | AHFSYITNGD | | | | | |
| | | ASTKGPAWLH | | | | | |
| | | EKIGDA | | | | | |
| G9BY57 | 20 | MDGVLWRVR | LCC | PF12695; | IPR029058; | 3.1.1.101; | Polyesterase- |
| | | TAALMAALLA | | | IPR029059; | 3.1.1.74 | lipase- |
| | | LAAWALVWA | | | | | cutinase |
| | | SPSVEAQSNPY | | | | | |
| | | QRGPNPTRSA | | | | | |
| | | LTADGPFSVA | | | | | |
| | | TYTVSRLSVSG | | | | | |
| | | FGGGVIYYPT | | | | | |
| | | GTSLTFGGIAM | | | | | |
| | | SPGYTADASSL | | | | | |
| | | AWLGRRLASH | | | | | |
| | | GFVVLVINTNS | | | | | |
| | | RFDYPDSRAS | | | | | |
| | | QLSAALNYLR | | | | | |
| | | TSSPSAVRARL | | | | | |
| | | DANRLAVAGH | | | | | |
| | | SMGGGGTLRI | | | | | |
| | | AEQNPSLKAA | | | | | |
| | | VPLTPWHTDK | | | | | |
| | | TFNTSVPVLIV | | | | | |
| | | GAEADTVAPV | | | | | |
| | | SQHAIPFYQNL | | | | | |
| | | PSTTPKVYVEL | | | | | |
| | | DNASHFAPNS | | | | | |
| | | NNAAISVYTIS | | | | | |
| | | WMKLWVDND | | | | | |
| | | TRYRQFLCNV | | | | | |
| | | NDPALSDFRT | | | | | |
| | | NNRHCQ | | | | | |
| H6WX58 | 21 | MANPYERGPN | Thh_Est | PF12146; | IPR029058; | | Polyesterase- |
| | | PTNSSIEALRG | | | IPR022742; | | lipase- |
| | | PFRVDEERVSR | | | | | cutinase |
| | | LQARGFGGGT | | | | | |
| | | IYYPTDNNTFG | | | | | |
| | | AVAISPGYTGT | | | | | |
| | | QSSISWLGERL | | | | | |
| | | ASHGFVVMTI | | | | | |
| | | DTNTTLDQPD | | | | | |
| | | SRASQLDAAL | | | | | |
| | | DYMVEDSSYS | | | | | |
| | | VRNRIDSSRLA | | | | | |
| | | AMGHSMGGG | | | | | |
| | | GTLRLAERRP | | | | | |
| | | DLQAAIPLTP | | | | | |
| | | WHTDKTWGS | | | | | |
| | | VRVPTLIIGAE | | | | | |
| | | NDTIASVRSHS | | | | | |
| | | EPFYNSLPGSL | | | | | |
| | | DKAYLELDGA | | | | | |
| | | SHFAPNLSNTT | | | | | |
| | | LAKYSISWLKR | | | | | |
| | | FVDDDTRYTQ | | | | | |
| | | FLCPGPSTGW | | | | | |
| | | GSDVEEYRST | | | | | |
| | | CPF | | | | | |
| P00590 | 22 | MKFFALTTLL | FsC | PF01083; | IPR029058; | 3.1.1.74 | Cutinase |
| | | AATASALPTS | | | IPR000675; | | |
| | | NPAQELEARQ | | | IPR043580; | | |
| | | LGRTTRDDLIN | | | IPR043579; | | |
| | | GNSASCRDVIF | | | IPR011150; | | |
| | | IYARGSTETGN | | | | | |
| | | LGTLGPSIASN | | | | | |
| | | LESAFGKDGV | | | | | |
| | | WIQGVGGAYR | | | | | |
| | | ATLGDNALPR | | | | | |
| | | GTSSAAIREML | | | | | |
| | | GLFQQANTKC | | | | | |
| | | PDATLIAGGYS | | | | | |
| | | QGAALAAASI | | | | | |
| | | EDLDSAIRDKI | | | | | |
| | | AGTVLFGYTK | | | | | |
| | | NLQNRGRIPN | | | | | |
| | | YPADRTKVFC | | | | | |
| | | NTGDLVCTGS | | | | | |
| | | LIVAAPHLAY | | | | | |
| | | GPDARGPAPE | | | | | |
| | | FLIEKVRAVRG | | | | | |
| | | SA | | | | | |
| P19833 | 23 | MFIMIKKSELA | MoPE | | IPR029058; | 3.1.1.3 | Polyesterase- |
| | | KAIIVTGALVF | | | | | lipase- |
| | | SIPTLAEVTLS | | | | | cutinase |
| | | ETTVSSIKSEA | | | | | |
| | | TVSSTKKALP | | | | | |
| | | ATPSDCIADSK | | | | | |
| | | ITAVALSDTRD | | | | | |
| | | NGPFSIRTKRIS | | | | | |
| | | RQSAKGFGGG | | | | | |
| | | TIHYPTNASGC | | | | | |
| | | GLLGAIAVVP | | | | | |
| | | GYVSYENSIK | | | | | |
| | | WWGPRLASW | | | | | |
| | | GFVVITINTNSI | | | | | |
| | | YDDPDSRAAQ | | | | | |
| | | LNAALDNMIA | | | | | |
| | | DDTVGSMIDP | | | | | |
| | | KRLGAIGWSM | | | | | |
| | | GGGGALKLAT | | | | | |
| | | ERSTVRAIMPL | | | | | |
| | | APYHDKSYGE | | | | | |
| | | VKTPTLVIACE | | | | | |
| | | DDRIAETKKY | | | | | |
| | | ANAFYKNAIG | | | | | |
| | | PKMKVEVNN | | | | | |
| | | GSHFCPSYRFN | | | | | |
| | | EILLSKPGIAW | | | | | |
| | | MQRYINNDTR | | | | | |
| | | FDKFLCANEN | | | | | |
| | | YSKSPRISAYD | | | | | |
| | | YKDCP | | | | | |
| Q2MDG4 | 24 | MKGIAALVTA | SterylEst | PF00135; | IPR029058; | 3.1.1.- | Funal_carboxy- |
| | | ALLGRAVAAP | | | IPR002018; | | lesterase_ |
| | | PEPPTNVVEKR | | | IPR019826; | | lipase |
| | | AAPTVEISTGT | | | IPR019819; | | |
| | | IVGTTRLATEA | | | | | |
| | | FNGIPFALPPV | | | | | |
| | | GQLRLKPPVR | | | | | |
| | | LNSSLGVFDAS | | | | | |
| | | GIAPACPQFLA | | | | | |
| | | DSDSNEFLAQ | | | | | |
| | | VINTVTGLPFF | | | | | |
| | | QKALKISEDCL | | | | | |
| | | TINVIRPKGTK | | | | | |
| | | AGDKLPVLFW | | | | | |
| | | IYGGGFELGW | | | | | |
| | | SSMYDGGPLV | | | | | |
| | | TNAIGFGKPFI | | | | | |
| | | FVAVNYRVAG | | | | | |
| | | FGFMPGKEILA | | | | | |
| | | DGAANLGHLD | | | | | |
| | | QRMGLEWVA | | | | | |
| | | DNIAAFGGDP | | | | | |
| | | DKVTIWGESA | | | | | |
| | | GAISVLNQMA | | | | | |
| | | LFDGDHTYKG | | | | | |
| | | KPLFRGAIMNS | | | | | |
| | | GSIVPADPVDC | | | | | |
| | | PKGQEIYDQV | | | | | |
| | | VAKAGCAGAS | | | | | |
| | | DTLACLRELP | | | | | |
| | | YEKFLDAANS | | | | | |
| | | VPAILSYNSVA | | | | | |
| | | LSYLPRPDGK | | | | | |
| | | VLTKSPDVLIK | | | | | |
| | | EGKYAAVPMII | | | | | |
| | | GDQEDEGTLF | | | | | |
| | | SLFQPNITNSE | | | | | |
| | | KLVTYLKDLF | | | | | |
| | | FHGASREQLE | | | | | |
| | | GLVDRYPARIS | | | | | |
| | | AGSPFRTGLLN | | | | | |
| | | EIYPGFKRLAA | | | | | |
| | | ILGDLVFTLTR | | | | | |
| | | RVFLEDATRV | | | | | |
| | | NPTVPAWSYL | | | | | |
| | | ASYDYGTPILG | | | | | |
| | | TFHGSDLLQV | | | | | |
| | | FFGILPNYASR | | | | | |
| | | SIQSYYANFVY | | | | | |
| | | NLDPNDASGG | | | | | |
| | | TSGKSKVAEE | | | | | |
| | | WPRWTADDR | | | | | |
| | | TLIQFFANRNG | | | | | |
| | | YLKDDFRSEA | | | | | |
| | | AEYIGAHVDY | | | | | |
| | | LHI | | | | | |
| Q6A0I3 | 25 | MAVMTPRRER | BTA-2 | PF12740; | IPR029058; | 3.1.1.101; | Polyesterase- |
| | | SSLLSRALRFT | | | IPR041127; | 3.1.1.74 | lipase- |
| | | AAAATALVTA | | | | | cutinase |
| | | VSLAAPAHAA | | | | | |
| | | NPYERGPNPT | | | | | |
| | | DALLEARSGPF | | | | | |
| | | SVSEERASRFG | | | | | |
| | | ADGFGGGTIY | | | | | |
| | | YPRENNTYGA | | | | | |
| | | VAISPGYTGTQ | | | | | |
| | | ASVAWLGKRI | | | | | |
| | | ASHGFVVITID | | | | | |
| | | TNTTLDQPDS | | | | | |
| | | RARQLNAALD | | | | | |
| | | YMINDASSAV | | | | | |
| | | RSRIDSSRLAV | | | | | |
| | | MGHSMGGGG | | | | | |
| | | SLRLASQRPDL | | | | | |
| | | KAAIPLTPWH | | | | | |
| | | LNKNWSSVRV | | | | | |
| | | PTLIIGADLDTI | | | | | |
| | | APVLTHARPF | | | | | |
| | | YNSLPTSISKA | | | | | |
| | | YLELDGATHF | | | | | |
| | | APNIPNKIIGK | | | | | |
| | | YSVAWLKRFV | | | | | |
| | | DNDTRYTQFL | | | | | |
| | | CPGPRDGLFG | | | | | |
| | | EVEEYRSTCPF | | | | | |
| R4YKL9 | 26 | MNKSILKKLSF | PET5 | PF01738; | IPR029058; | | Polyesterase- |
| | | GTSVLLVSMN | | | IPR002925; | | lipase- |
| | | ALSWTPSPTPN | | | | | cutinase |
| | | PDPIPDPTPCQ | | | | | |
| | | DDCDFTRGPN | | | | | |
| | | PTPSSLEASTG | | | | | |
| | | PYSVATRSVA | | | | | |
| | | SSVSGFGGGTL | | | | | |
| | | HYPTNTTGTM | | | | | |
| | | GALAVVPGFLL | | | | | |
| | | QESSIDFWGPK | | | | | |
| | | LASHGFVVITI | | | | | |
| | | SANSGFDQPA | | | | | |
| | | SRATQLGRAL | | | | | |
| | | DYVINQSNGS | | | | | |
| | | NSPISGMVDTT | | | | | |
| | | RLGVVGWSM | | | | | |
| | | GGGGALQLAS | | | | | |
| | | GDRLSAAIPIA | | | | | |
| | | PWNQGGNRFD | | | | | |
| | | QIETPTLVIAC | | | | | |
| | | ENDVVASVNS | | | | | |
| | | HASPFYNRIPS | | | | | |
| | | TTDKAYLEIN | | | | | |
| | | GGSHFCANDG | | | | | |
| | | GSIGGLLGKY | | | | | |
| | | GVSWMKRFID | | | | | |
| | | NDLRYDAFLC | | | | | |
| | | GPDHAANRSV | | | | | |
| | | SEYRDTCNY | | | | | |
| W0TJ64 | 27 | MRIRRQAGTG | Cut190 | PF03403; | IPR029058; | 3.1.1.74 | Polyesterase- |
| | | ARASMARAIG | | | | | lipase- |
| | | VMTTALAVLV | | | | | cutinase |
| | | GAVGGVAGA | | | | | |
| | | EVSTAQDNPY | | | | | |
| | | ERGPDPTEDSI | | | | | |
| | | EAIRGPFSVAT | | | | | |
| | | ERVSSFASGFG | | | | | |
| | | GGTIYYPRETD | | | | | |
| | | EGTFGAVAVA | | | | | |
| | | PGFTASQGSM | | | | | |
| | | SWYGERVASQ | | | | | |
| | | GFIVFTIDTNT | | | | | |
| | | RLDQPGQRGR | | | | | |
| | | QLLAALDYLV | | | | | |
| | | ERSDRKVRER | | | | | |
| | | LDPNRLAVMG | | | | | |
| | | HSMGGGGSLE | | | | | |
| | | ATVMRPSLKA | | | | | |
| | | SIPLTPWNLDK | | | | | |
| | | TWGQVQVPTF | | | | | |
| | | IIGAELDTIASV | | | | | |
| | | RTHAKPFYESL | | | | | |
| | | PSSLPKAYME | | | | | |
| | | LDGATHFAPNI | | | | | |
| | | PNTTIAKYVIS | | | | | |
| | | WLKRFVDEDT | | | | | |
| | | RYSQFLCPNPT | | | | | |
| | | DRAIEEYRSTC | | | | | |
| | | PY | | | | | |
| X0BTD8 | 28 | MKFSIISTLLA | FoCut5a | PF01083; | IPR029058; | | Cutinase |
| | | ATASALPAGQ | | | IPR000675; | | |
| | | DAAALEARQL | | | IPR043580; | | |
| | | GGSITRNDLA | | | IPR043579; | | |
| | | NGNSGSCPGVI | | | IPR011150; | | |
| | | FIYARGSTESG | | | | | |
| | | NLGTLGPRVA | | | | | |
| | | SKLEAKYGKN | | | | | |
| | | GVWIQGVGGA | | | | | |
| | | YRATLGDNAL | | | | | |
| | | PRGTSSAAIRE | | | | | |
| | | MLGHFSDANQ | | | | | |
| | | KCPDAVLIAG | | | | | |
| | | GYSQGAALAA | | | | | |
| | | ASVTDVDAGI | | | | | |
| | | REKIAGVVLF | | | | | |
| | | GYTKNLQNRG | | | | | |
| | | KIPSYPEDRTK | | | | | |
| | | VFCNTGDLVC | | | | | |
| | | TGSLIVAAPHL | | | | | |
| | | AYQSAASGAA | | | | | |
| | | PEFLIQKADAA | | | | | |
| | | GAA | | | | | |
| A0A031MKR8 | 29 | MINKNLSQSLL | PauzCut | PF01738 | IPR029058; | | Polyesterase- |
| | | AMMAAGALL | | | IPR002925 | | lipase- |
| | | LSSSAFAVNPP | | | | | cutinase |
| | | TDGPTDPDQA | | | | | |
| | | YERGPDPSVA | | | | | |
| | | FLEAPTGPHSV | | | | | |
| | | RTSRVSGLVS | | | | | |
| | | GFGGGTIHYPT | | | | | |
| | | GTTGTMAAIV | | | | | |
| | | VIPGFVSAESSI | | | | | |
| | | EWWGPKLAS | | | | | |
| | | HGFVVMTIDT | | | | | |
| | | NTGFDQPPSR | | | | | |
| | | ARQINNALDY | | | | | |
| | | LVSQNTSRTSP | | | | | |
| | | VNGMIDTERL | | | | | |
| | | GVIGWSMGGG | | | | | |
| | | GTLRVASEGRI | | | | | |
| | | KAAIPLAPWD | | | | | |
| | | TTRFRGVQAP | | | | | |
| | | TLIFACESDLIA | | | | | |
| | | PVRSHASPFYN | | | | | |
| | | QLPDDIDKAY | | | | | |
| | | VEINNGSHYC | | | | | |
| | | ANGGGLNND | | | | | |
| | | VLSRFGVSWM | | | | | |
| | | KRFLDNDTRY | | | | | |
| | | SQFLCGPNHES | | | | | |
| | | DRNISEYRGN | | | | | |
| | | CPY | | | | | |
| A0A078MGG8 | 30 | MINKKLSQTL | PsCut | PF01738 | IPR029058; | | Polyesterase- |
| | | LSMAAAGAL | PET6, | | IPR002925 | | lipase- |
| | | MFSASVFATN | | | | | cutinase |
| | | PPTDEPTNPGQ | | | | | |
| | | SYERGPDPTV | | | | | |
| | | AFLEASSGPYS | | | | | |
| | | VRTSRVSGLV | | | | | |
| | | SGFGGGTIHYP | | | | | |
| | | TGTTGTMAAI | | | | | |
| | | VVIPGFVSAES | | | | | |
| | | SIDWWGPKLA | | | | | |
| | | SHGFVVMTID | | | | | |
| | | TNTGFDQPPSR | | | | | |
| | | ARQINNALDY | | | | | |
| | | LVDQNSSRTSP | | | | | |
| | | VNGMIDTERL | | | | | |
| | | GVIGWSMGGG | | | | | |
| | | GTLRVASEGRI | | | | | |
| | | KAAIPLAPWD | | | | | |
| | | TTRFSGVQAPT | | | | | |
| | | LIFACESDLIAP | | | | | |
| | | VRSHASPFYN | | | | | |
| | | QLPNDIDKAY | | | | | |
| | | VEINNGSHYC | | | | | |
| | | ANGGGLNND | | | | | |
| | | VLSRFGVSWM | | | | | |
| | | KRFLDNDTRY | | | | | |
| | | SQFLCGPRHES | | | | | |
| | | DRKISEYRGN | | | | | |
| | | CPY | | | | | |
| A0A1Z2SIQ1 | 31 | MMNVLTKCK | BSQ33_03270 | | IPR029058; | | Polyesterase- |
| | | LALGIIAIFFSL | | | | | lipase- |
| | | PSFAVPCSDCS | | | | | cutinase |
| | | NGFERGQVPR | | | | | |
| | | VDQLESSRGP | | | | | |
| | | YSVKTINVSRL | | | | | |
| | | ARGFGGGTIH | | | | | |
| | | YSTESGGQQGI | | | | | |
| | | LAVVPGYVSLE | | | | | |
| | | GSIKWWGPRL | | | | | |
| | | ASWGFTVITID | | | | | |
| | | TNTIYDQPDSR | | | | | |
| | | ASQLSAAIDY | | | | | |
| | | VIDKGNDRSSP | | | | | |
| | | IYGLVDPNRV | | | | | |
| | | GVIGWSMGGG | | | | | |
| | | GSLKLATDRKI | | | | | |
| | | DAVIPQAPWY | | | | | |
| | | LGLSRFSSITSP | | | | | |
| | | TMIIACQADV | | | | | |
| | | VAPVSVHASR | | | | | |
| | | FYNQIPGTTPK | | | | | |
| | | AYFEIALGSHF | | | | | |
| | | CANTGYPSEDI | | | | | |
| | | LGRNGVAWM | | | | | |
| | | KRFIDKDERYT | | | | | |
| | | QFLCGQNFDS | | | | | |
| | | SLRVSEYRDN | | | | | |
| | | CSYY | | | | | |
| E8U721 | 32 | MSNGYSACPA | DSM 21211, | PF01738 | IPR029058; | 3.1.1.3 | Polyesterase- |
| | | PYDHRGMRK | PET1/ | | IPR002925 | | lipase- |
| | | LLAACGTTTST | DmPETase | | | | cutinase |
| | | LTTPAVTPQTL | | | | | |
| | | SGAATNPYER | | | | | |
| | | GPAPTTALLEA | | | | | |
| | | ARGPYATAST | | | | | |
| | | TVPRSSVSDFG | | | | | |
| | | GATIYYPTSTA | | | | | |
| | | DGTFGGVAISP | | | | | |
| | | GYTGTQASVA | | | | | |
| | | WLGPRLASHG | | | | | |
| | | FVVIVIDTLSR | | | | | |
| | | YDYPSSRGDQ | | | | | |
| | | LRAALRYLTT | | | | | |
| | | SSAVRTRVDA | | | | | |
| | | TRLAVMGHS | | | | | |
| | | MGGGGALEA | | | | | |
| | | AKDNPALKAA | | | | | |
| | | IPLTPWNTDKS | | | | | |
| | | WPELTTPTLIF | | | | | |
| | | GAQNDSVAPV | | | | | |
| | | SSHAIPFYTSL | | | | | |
| | | ASTLPKAYLEL | | | | | |
| | | RGASHGAPTS | | | | | |
| | | TNTTIAKYAV | | | | | |
| | | AWLKRFEDAD | | | | | |
| | | RRYDPFLCPTP | | | | | |
| | | AVSTLLSDARS | | | | | |
| | | TCPFN | | | | | |
| P41365 | 33 | MKLLSLTGVA | CalB; | | IPR029058 | 3.1.1.3 | Canar_LipB |
| | | GVLATCVAAT | lipase B | | | | |
| | | PLVKRLPSGSD | | | | | |
| | | PAFSQPKSVLD | | | | | |
| | | AGLTCQGASP | | | | | |
| | | SSVSKPILLVP | | | | | |
| | | GTGTTGPQSF | | | | | |
| | | DSNWIPLSTQL | | | | | |
| | | GYTPCWISPPP | | | | | |
| | | FMLNDTQVNT | | | | | |
| | | EYMVNAITAL | | | | | |
| | | YAGSGNNKLP | | | | | |
| | | VLTWSQGGLV | | | | | |
| | | AQWGLTFFPSI | | | | | |
| | | RSKVDRLMAF | | | | | |
| | | APDYKGTVLA | | | | | |
| | | GPLDALAVSA | | | | | |
| | | PSVWQQTTGS | | | | | |
| | | ALTTALRNAG | | | | | |
| | | GLTQIVPTTNL | | | | | |
| | | YSATDEIVQPQ | | | | | |
| | | VSNSPLDSSYL | | | | | |
| | | FNGKNVQAQA | | | | | |
| | | VCGPLFVIDHA | | | | | |
| | | GSLTSQFSYVV | | | | | |
| | | GRSALRSTTG | | | | | |
| | | QARSADYGIT | | | | | |
| | | DCNPLPANDL | | | | | |
| | | TPEQKVAAAA | | | | | |
| | | LLAPAAAAIV | | | | | |
| | | AGPKQNCEPD | | | | | |
| | | LMPYARPFAV | | | | | |
| | | GKRTCSGIVTP | | | | | |
| Q47RJ7 | 34 | MPPHAARPGP | Tfu_0882 | PF12740 | IPR029058; | 3.1.1.101; | Polyesterase- |
| | | AQNRRGRAM | | | IPR041127 | 3.1.1.74 | lipase- |
| | | AVITPRRERSS | | | | | cutinase |
| | | LLSRALRFTAA | | | | | |
| | | AATALVTAVS | | | | | |
| | | LAAPAHAANP | | | | | |
| | | YERGPNPTDA | | | | | |
| | | LLEARSGPFSV | | | | | |
| | | SEERASRFGA | | | | | |
| | | DGFGGGTIYY | | | | | |
| | | PRENNTYGAV | | | | | |
| | | AISPGYTGTQA | | | | | |
| | | SVAWLGERIA | | | | | |
| | | SHGFVVITIDT | | | | | |
| | | NTTLDQPDSR | | | | | |
| | | ARQLNAALDY | | | | | |
| | | MINDASSAVR | | | | | |
| | | SRIDSSRLAVM | | | | | |
| | | GHSMGGGGTL | | | | | |
| | | RLASQRPDLK | | | | | |
| | | AAIPLTPWHL | | | | | |
| | | NKNWSSVRVP | | | | | |
| | | TLIIGADLDTIA | | | | | |
| | | PVLTHARPFY | | | | | |
| | | NSLPTSISKAY | | | | | |
| | | LELDGATHFA | | | | | |
| | | PNIPNKIIGKYS | | | | | |
| | | VAWLKRFVD | | | | | |
| | | NDTRYTQFLC | | | | | |
| | | PGPRDGLFGE | | | | | |
| | | VEEYRSTCPF | | | | | |
| Q8RR62 | 35 | MHLPRSRWDI | AdCut | PF01738 | IPR029058; | | Polyesterase- |
| | | PFKEETTMTH | | | IPR002925 | | lipase- |
| | | HFSVRALLAA | | | | | cutinase |
| | | GALLASAAVS | | | | | |
| | | AQTNPYERGP | | | | | |
| | | APTTSSLEASR | | | | | |
| | | GPFSYQSFTVS | | | | | |
| | | RPSGYRAGTV | | | | | |
| | | YYPTNAGGPV | | | | | |
| | | GAIAIVPGFTA | | | | | |
| | | RQSSINWWGP | | | | | |
| | | RLASHGFVVIT | | | | | |
| | | IDTNSTLDQPD | | | | | |
| | | SRSRQQMAAL | | | | | |
| | | SQVATLSRTSS | | | | | |
| | | SPIYNKVDTSR | | | | | |
| | | LGVMGWSMG | | | | | |
| | | GGGSLISARNN | | | | | |
| | | PSIKAAAPQAP | | | | | |
| | | WSASKNFSSL | | | | | |
| | | TVPTLILACEN | | | | | |
| | | DTIAPVNQHA | | | | | |
| | | DTFYDSMSRN | | | | | |
| | | PREFLEINNGS | | | | | |
| | | HSCANSGNSN | | | | | |
| | | QALLGKKGVA | | | | | |
| | | WMKRFMDND | | | | | |
| | | RRYTSFACSNP | | | | | |
| | | NSYNVSDFRV | | | | | |
| | | AACN | | | | | |
| W6R2Y2 | 36 | MRAWWLSGA | PpEst (tesA) | PF13472 | IPR013830; | 3.1.2.- | |
| | | LALMFWAQG | | | IPR036514 | | |
| | | AVAGTLLVVG | | | | | |
| | | DSISAAFGLDS | | | | | |
| | | RQGWVALLEK | | | | | |
| | | RLSEEGFEHSV | | | | | |
| | | VNASISGDTSA | | | | | |
| | | GGAARLSALL | | | | | |
| | | AEHKPELVIIE | | | | | |
| | | LGGNDGLRGQ | | | | | |
| | | PPAQLQQNLA | | | | | |
| | | SMVEQSQQAG | | | | | |
| | | AKVLLLGMKL | | | | | |
| | | PPNYGVRYTT | | | | | |
| | | AFAQVFTDLA | | | | | |
| | | EQKQVSLVPFF | | | | | |
| | | LEGVGGVPGM | | | | | |
| | | MQADGIHPAE | | | | | |
| | | AAQEILLDNV | | | | | |
| | | WPTLKPML | | | | | |
| LT571442.1 | 37 | MENPYERGPD | PHL3 | | | | |
| | | PTESSIEAVRG | | | | | |
| | | PFAVAQTTVS | | | | | |
| | | RLQADGFGGG | | | | | |
| | | TIYYPTDTSQG | | | | | |
| | | TFGAVAISPGF | | | | | |
| | | TAGQESIAWL | | | | | |
| | | GPRIASQGFVV | | | | | |
| | | ITIDTITRLDQP | | | | | |
| | | DSRGRQLQAA | | | | | |
| | | LDHLRTNSVV | | | | | |
| | | RNRIDPNRMA | | | | | |
| | | VMGHSMGGG | | | | | |
| | | GALSAAANNT | | | | | |
| | | SLEAAIPLQG | | | | | |
| | | WHTRKNWSS | | | | | |
| | | VRTPTLVVGA | | | | | |
| | | QLDTIAPVSSH | | | | | |
| | | SEAFYNSLPSD | | | | | |
| | | LDKAYMELRG | | | | | |
| | | ASHFVSNTPDT | | | | | |
| | | TTAKYSIAWL | | | | | |
| | | KRFVDNDLRY | | | | | |
| | | EQFLCPAPDDF | | | | | |
| | | AISEYRATCPF | | | | | |
| LT571446.1 | 38 | MANPYERGPD | PHL7 | | | | |
| | | PTESSIEAVRG | | | | | |
| | | PFAVAQTTVS | | | | | |
| | | RLQADGFGGG | | | | | |
| | | TIYYPTDTSQG | | | | | |
| | | TFGAVAISPGF | | | | | |
| | | TAGQESIAWL | | | | | |
| | | GPRIASQGFVV | | | | | |
| | | ITIDTITRLDQP | | | | | |
| | | DSRGRQLQAA | | | | | |
| | | LDHLRTNSVV | | | | | |
| | | RNRIDPNRMA | | | | | |
| | | VMGHSMGGG | | | | | |
| | | GALSAAANNT | | | | | |
| | | SLEAAIPLQG | | | | | |
| | | WHTRKNWSS | | | | | |
| | | VRTPTLVVGA | | | | | |
| | | QLDTIAPVSSH | | | | | |
| | | SEAFYNSLPSD | | | | | |
| | | LDKAYMELRG | | | | | |
| | | ASHLVSNTPD | | | | | |
| | | TTTAKYSIAW | | | | | |
| | | LKRFVDDDLR | | | | | |
| | | YEQFLCPAPD | | | | | |
| | | DFAISEYRSTC | | | | | |
| | | PF | | | | | |

In some embodiments, the polymer-degrading enzyme is a HiC. In some embodiments, the amino acid sequence of the HiC enzyme is set forth as: SEQ ID NO: 1 or a fragment thereof. In some embodiments, the polymer-degrading enzyme is a variant of HiC having an insertion, deletion, or amino acid substitution at any one or more of the following positions: 1, 2, 5, 43, 55, 79, 115, 161, 181, 182, G8, S116, S119, A4, T29, L167, S48, N15, A88, N91, A130, T166, Q139, 1169, 1178 or R189 compared with the amino acid sequence of the HiC enzyme is set forth as: SEQ ID NO: 1. In some embodiments, the polymer-degrading enzyme is a variant of HiC having an amino acid substitution at up to 1, 2, 3, 4, 5, 6, 7, 8, 9, 10, 11, 12, 13, 14, 15, 16, 17, 18, 19, or 20 sites selected from the previous list. In some embodiments, the polymer-degrading enzyme is a variant of HiC, in which the variant of HiC has at least 85%, 86%, 87%, 88%, 89%, 90%, 91%, 92%, 93%, 94%, 95%, 96%, 97%, 98%, or 99% identity compared with the amino acid sequence of the HiC enzyme is set forth as: SEQ ID NO: 1.

In some embodiments, the polymer-degrading enzyme is a leaf-branch compost cutinase (LCC). In some embodiments, the amino acid sequence of the LCC enzyme is set forth as: SEQ ID NO: 20 or a fragment thereof. In some embodiments, the polymer-degrading enzyme is a variant of LCC having an insertion, deletion, or amino acid substitution at any one or more of the following positions: D238, S283, E208, L237, N239, A207, A244, V63, S64, R65, L66, S67, V68, S69, G70, F71, G72, G73, G74, A138, L117, G88, L139, L142, L154, A156, L159, 189, M91, L105, L109, A162, V185, L187, L203, V205, P231, V233, V235, V254, Y255, T256, S258, W259, M260, L274, T287, N288, H291, S36, Y39, Q40, R41, N44, S48, T51, S57, T60, Y61, Y78, S83, T85, R107, S133, N140, R143, S148, N157, S180, K182, T195, N197, S216, Q224, N225, S228, T229, S247, N248, N266, T268, R271, Q272, N276, N278, N289, R290, Q293, V2121, Y127G, Y127P, F2431, F243W, T96M, V2051, D238C, S283C, E208R, E208A, N239D, or L237R compared with the amino acid sequence of the LCC enzyme is set forth as: SEQ ID NO: 20. For example, in some embodiments, the polymer-degrading enzyme is a variant of LCC having one or more of the following substitutions F243I, D238C, S283C, and Y127G compared with the amino acid sequence of the LCC enzyme is set forth as: SEQ ID NO: 20. In some embodiments, the polymer-degrading enzyme comprises or consists of an amino acid sequence corresponding to positions 36 to 258 of SEQ ID NO: 20. In some embodiments, the polymer-degrading enzyme comprises or consists of an amino acid sequence corresponding to positions 36 to 258 of SEQ ID NO: 20 with an insertion, deletion, or amino acid substitutions at any one or more of the corresponding positions of the previous lists. In some embodiments, the polymer-degrading enzyme is a variant of LCC having an amino acid substitution at up to 1, 2, 3, 4, 5, 6, 7, 8, 9, 10, 11, 12, 13, 14, 15, 16, 17, 18, 19, or 20 sites selected from the previous list. In some embodiments, the polymer-degrading enzyme is a variant of LCC, in which the variant of LCC has at least 85%, 86%, 87%, 88%, 89%, 90%, 91%, 92%, 93%, 94%, 95%, 96%, 97%, 98%, or 99% identity compared with the amino acid sequence of the LCC enzyme is set forth as: SEQ ID NO: 20.

In some embodiments, polymer-degrading enzymes can be engineered according to information in the following literary publications which are herein incorporated by reference in their entirety for all purposes: Dombkowski A, Sultana K Z, Craig D. Protein disulfide engineering. FEBS Letters Volume 588, Issue 2, 206-212. 2014; Liu Q, Xun G, Feng Y. The state-of-the-art strategies of protein engineering for enzyme stabilization. Biotechnol Adv. 2019 July-August; 37(4):530-537. doi: 10.1016/j.biotechadv.2018.10.011. Epub 2018 Oct. 26. PMID: 31138425; Federica Rigoldi, Stefano Donini, Alberto Redaelli, Emilio Parisini, Alfonso Gautieri; Review: Engineering of thermostable enzymes for industrial applications. APL Bioeng. 1 Mar. 2018; 2 (1): 011501. https://doi.org/10.1063/1.4997367; Chen, K., Arnold, F. H. Engineering new catalytic activities in enzymes. Nat Catal 3, 203-213 (2020). https://doi.org/10.1038/41929-019. 0385-5; Robert Chapman and Martina H. Stenzel. All Wrapped up: Stabilization of Enzymes within Single Enzyme Nanoparticles. Journal of the American Chemical Society 2019 141 (7), 2754-2769. DOI: 10.1021/jacs.8b10338; Spence M, Kaczmarski J, Saunders J, Jackson C. Ancestral sequence reconstruction for protein engineers. Current Opinion in Structural Biology, Volume 69. 2021; Raquel A. Rocha, Robert E. Speight, and Colin Scott. Engineering Enzyme Properties for Improved Biocatalytic Processes in Batch and Continuous Flow.Organic Process Research & Development 2022 26 (7), 1914-1924. DOI: 10.1021/acs.oprd. 1c00424; Chowdhury, R, Maranas, C D. From directed evolution to computational enzyme engineering-A review. AICHE J. 2020; 66:e16847.https://doi.org/10.1002/aic. 16847; and Ferreira P, Fernandes P A, Ramos M J. Modern computational methods for rational enzyme engineering. Chem Catalysis, Volume 2, Issue 10, 2481-2498. 2022.

In some embodiments, a polymer-degrading enzyme comprises one or more conservative amino acid substitutions relative to a reference sequence. Such conservative substitutions of amino acids include substitutions made amongst amino acids within the following groups: (a) M, I, L, V; (b) F, Y, W; (c) K, R, H; (d) A, G; (c) S, T; (f) Q, N; and (g) E, D. In general, a conservative amino acid substitution refers to an amino acid substitution that does not alter the relative charge or size characteristics of the protein in which the amino acid substitution is made. In some embodiments, the polymer-degrading enzyme comprises at least 1, 2, 3, 4, 5 or more amino acid substitutions within the active site of the enzyme. In some embodiments, the polymer-degrading enzyme comprises at least 1, 2, 3, 4, 5 or more amino acid substitutions outside the active site of the enzyme. In some embodiments, the polymer-degrading enzyme is a variant of an enzyme that comprises a substitution of one or more amino acids in or proximal to a divalent metal binding site of the enzyme with cystine amino acids to promote formation of a disulfide bridge, e.g., thereby increasing thermostability relative to the parent enzyme.

In some embodiments, exposing the pretreated polymeric material to the polymer-degrading enzyme occurs at a relatively high temperature. In some embodiments, exposing the pretreated polymeric material to the polymer-degrading enzyme occurs at a temperature close or higher than a glass transition temperature of the crystallizable polymer or copolymer. In some embodiments, exposing the pretreated polymeric material to the polymer-degrading enzyme occurs at a temperature in a range from a temperature that is 5° C., 10° C., 15° C., or 20° C. lower than a glass transition temperature of the crystallizable polymer or copolymer to a temperature of at least 95° C., 100° C., 105° C., 110° C., 115° C., or 120° C. In certain embodiments, the temperature is in a range from a temperature that is 15° C. less than a glass transition temperature of the crystallizable polymer or copolymer to a temperature of 120° C. In certain embodiments, the temperature is in a range from a temperature that is 10° C. lower than a glass transition temperature of the crystallizable polymer or copolymer to a temperature of 95° C.

In some embodiments, exposing the pretreated polymeric material to the polymer-degrading enzyme occurs at a temperature close or higher than a glass transition temperature of the crystallizable polymer or copolymer soaked up to equilibrium in water or in a buffer at a soaking temperature between room temperature and a temperature 20° C. above a glass transition temperature of the dry crystallizable polymer or copolymer.

In some embodiments, exposing the pretreated polymeric material to the polymer-degrading enzyme occurs at a temperature of at least 20° C., at least 30° C., at least 40° C., at least 50° C., at least 55° C., at least 60° C., at least 65° C., at least 70° C., at least 75° C., at least 80° C., at least 85° C., at least 90° C., at least 95° C., or at least 100° C. In certain embodiments, exposing the pretreated polymeric material to the polymer-degrading enzyme occurs at a temperature in a range from 20° C. to 40° C., 20° C. to 50° C., 20° C. to 60° C., 20° C. to 65° C., 20° C. to 70° C., 20° C. to 75° C., 20° C. to 80° C., 20° C. to 85° C., 20° C. to 90° C., 20° C. to 95° C., 20° C. to 100° C., 30° C. to 50° C., 30° C. to 60° C., 30° C. to 65° C., 30° C. to 70° C., 30° C. to 75° C., 30° C. to 80° C., 30° C. to 85° C., 30° C. to 90° C., 30° C. to 95° C., 30° C. to 100° C., 40° C. to 60° C., 40° C. to 65° C., 40° C. to 70° C., 40° C. to 75° C., 40° C. to 80° C., 40° C. to 85° C., 40° C. to 90° C., 40° C. to 95° C., 40° C. to 100° C., 50° C. to 60° C., 50° C. to 65° C., 50° C. to 70° C., 50° C. to 75° C., 50° C. to 80° C., 50° C. to 85° C., 50° C. to 90° C., 50° C. to 95° C., 50° C. to 100° C., 55° C. to 65° C., 55° C. to 70° C., 55° C. to 75° C., 55° C. to 80° C., 55° C. to 85° C., 55° C. to 90° C., 55° C. to 95° C., 55° C. to 100° C., 60° C. to 70° C., 60° C. to 75° C., 60° C. to 80° C., 60° C. to 85° C., 60° C. to 90° C., 60° C. to 95° C., 60° C. to 100° C., 65° C. to 75° C., 65° C. to 80° C., 65° C. to 85° C., 65° C. to 90° C., 65° C. to 95° C., 65° C. to 100° C., 70° C. to 80° C., 70° C. to 85° C., 70° C. to 90° C., 70° C. to 95° C., 70° C. to 100° C., 75° C. to 85° C., 75° C. to 90° C., 75° C. to 95° C., 75° C. to 100° C., 80° C. to 90° C., 80° C. to 95° C., 80° C. to 100° C., 85° C. to 95° C., 85° C. to 100° C., or 90° C. to 100° C.

In some embodiments, exposing the pretreated polymeric material to the polymer-degrading enzyme occurs at a pH suitable for enzymatic degradation. For example, exposing the pretreated polymeric material to the polymer-degrading enzyme may occur in an environment having a pH of between 1 and 14, between 4 and 12, between 6 and 11, between 6 and 8, or between 7 and 9. The pH may be modulated in any of a variety of manners, such as via the addition of an acid and/or a base (e.g., at desired intervals during the enzymatic degradation process), a buffer having a particular buffer concentration, etc. Non-limiting examples of a buffer include sodium phosphate, potassium phosphate, glycine buffer, and Tris-HCl.

In some embodiments, exposing the pretreated polymeric material to the polymer-degrading enzyme occurs for a duration of at least 10 minutes, at least 30 minutes, at least 60 minutes, at least 90 minutes, at least 2 hours, at least 4 hours, at least 6 hours, at least 8 hours, at least 10 hours, at least 12 hours, at least 1 day, at least 2 days, at least 3 days, or at least 4 days. In certain embodiments, exposing the pretreated polymer to the polymer-degrading enzyme occurs for a duration in a range from 10 to 30 minutes, 10 to 60 minutes, 10 to 90 minutes, 10 minutes to 2 hours, 10 minutes to 4 hours, 10 minutes to 6 hours, 10 minutes to 8 hours, 10 minutes to 10 hours, 10 minutes to 12 hours, 10 minutes to 1 day, 10 minutes to 2 days, 10 minutes to 3 days, 10 minutes to 4 days, 30 to 60 minutes, 30 to 90 minutes, 30 minutes to 2 hours, 30 minutes to 4 hours, 30 minutes to 6 hours, 30 minutes to 8 hours, 30 minutes to 10 hours, 30 minutes to 12 hours, 30 minutes to 1 day, 30 minutes to 2 days, 30 minutes to 3 days, 30 minutes to 4 days, 1 to 2 hours, 1 to 4 hours, 1 to 6 hours, 1 to 8 hours, 1 to 10 hours, 1 to 12 hours, 1 hour to 1 day, 1 hour to 2 days, 1 hour to 3 days, 1 hour to 4 days, 2 to 4 hours, 2 to 6 hours, 2 to 8 hours, 2 to 10 hours, 2 to 12 hours, 2 hours to 1 day, 2 hours to 2 days, 2 hours to 3 days, 2 hours to 4 days, 4 to 6 hours, 4 to 8 hours, 4 to 10 hours, 4 to 12 hours, 4 hours to 1 day, 4 hours to 2 days, 4 hours to 3 days, 4 hours to 4 days, 6 to 8 hours, 6 to 10 hours, 6 to 12 hours, 6 hours to 1 day, 6 hours to 2 days, 6 hours to 3 days, 6 hours to 4 days, 8 to 10 hours, 8 to 12 hours, 8 hours to 1 day, 8 hours to 2 days, 8 hours to 3 days, 8 hours to 4 days, 10 hours to 1 day, 10 hours to 2 days, 10 hours to 3 days, 10 hours to 4 days, 12 hours to 1 day, 12 hours to 2 days, 12 hours to 3 days, 12 hours to 4 days, 1 to 2 days, 1 to 3 days, 1 to 4 days, 2 to 4 days, or 3 to 4 days.

In some embodiments, exposing the pretreated polymeric material to the polymer-degrading enzyme for the duration results in a reaction yield of at least 15%, at least 20%, at least 25%, at least 30%, at least 40%, at least 50%, at least 60%, at least 70%, at least 80%, at least 90%, at least 95%, at least 99%, or about 100%. In some embodiments, exposing the pretreated polymeric material to the polymer-degrading enzyme for the duration results in a reaction yield in a range of 15-30%, 15-50%, 15-80%, 15-90%, 15-95%, 15-100%, 20-40%, 20-50%, 20-80%, 20-90%, 20-95%, 20-100%, 30-60%, 30-80%, 30-90%, 30-95%, 30-100%, 40-60%, 40-80%, 40-90%, 40-95%, 40-100%, 50-80%, 50-90%, 50-95%, 50-100%, 60-80%, 60-90%, 60-95%, 60-100%, 70-90%, 70-95%, 70-100%, 80-90%, 80-95%, 80-100%, or 90-100%.

In some embodiments, the pretreated polymeric material may have a relatively higher rate of enzyme degradation compared to untreated polymeric materials under otherwise identical conditions. In certain embodiments, a rate of enzymatic degradation per unit equivalent surface area of the pretreated polymeric material is at least 1.05 times, at least 1.1 times, at least 1.15 times, at least 1.5 times, at least 2 times, at least 3 times, at least 5 times, at least 10 times, at least 25 times, at least 50 times, at least 75 times, or more, and/or up to 100 times, up to 250 times, up to 500 times, or up to 1000 times, faster (or higher) than the rate of enzymatic degradation per unit equivalent surface area of the untreated crystallizable polymer or copolymer and/or the untreated polymeric material comprising the crystallizable polymer or copolymer, under otherwise identical conditions. Equivalent surface area is calculated as the surface area of a spherical particle having diameter equal to the (measured) average particle size of the corresponding crystallizable polymer or copolymer and/or the corresponding polymeric material comprising the crystallizable polymer or copolymer prior to or after the milling. Combinations of the above-referenced ranges are possible (e.g., at least 1.05 and less than or equal to 1000, at least 1.5 and less than or equal to 500, or at least 2 and less than or equal to 250). Other ranges are also possible. The rate of enzymatic degradation per unit surface area, in some embodiments, refers to the rate of enzymatic degradation per unit of surface area of the crystallizable polymer or copolymer that is accessible to the enzyme.

The rate of enzymatic degradation of the crystallizable polymer or copolymer may be measured via any of a variety of appropriate methods. For example, one of more products and/or byproducts from the enzymatic degradation (e.g., depolymerization) of the crystallizable polymer or copolymer may be measured using absorbance. In some cases, the concentration of byproducts and/or products may be correlated with the measured absorbance to determine the degree of enzymatic degradation. As an exemplary example, in embodiments in which the crystallizable polymer or copolymer comprises polyethylene terephthalate, the concentration of a specific byproduct, terephthalic acid, may be measured via absorbance and used to determine the degree of enzymatic degradation of the polymer. In some embodiments, the rate of enzymatic degradation of the crystallizable polymer or copolymer may be measured by high-performance liquid chromatography (HPLC), addition of base (titration), measurement of pH change, and/or measurement of remaining unreacted crystallizable polymer or copolymer.

Some aspects are directed to a material configured for enzymatic degradation. In some embodiments, the material configured for enzymatic degradation comprises a post-consumer and/or post-industrial polymeric material (PC/IPM). The PC/IPM may comprise polymeric material that has been used in one or more consumer products (e.g., food and beverage containers, packaging for health and beauty products, clothing, automotive components, etc.), industrial products (e.g., a product used in a manufacturing process), and/or industrial processes (e.g., waste from a manufacturing process). In some embodiments, the PC/IPM comprises one or more additives (e.g., dyes, plasticizers, catalysts, antioxidants). In some embodiments, the PC/IPM comprises one or more contaminants (e.g., paper fibers, adhesives, other polymers, etc.). In some cases, the PC/IPM is formed by mechanically processing (e.g., grinding, washing, drying, etc.) raw waste from one or more consumer products, industrial products, and/or industrial processes. In some cases, the PC/IPM is formed by chemically processing one or more components of raw waste from one or more consumer products, industrial products, and/or industrial processes. The PC/IPM may be identified and distinguished from virgin polymeric material by the presence (even in trace amounts) of one or more additives and/or contaminants, or reaction products thereof, which may be indicative of use in one or more consumer products, industrial products, and/or industrial processes.

In some embodiments, the PC/IPM comprises a crystallizable polymer or copolymer. The crystallizable polymer or copolymer may be any crystallizable polymer or copolymer described herein. In some cases, the crystallizable polymer or copolymer forms at least 50 wt. % of the PC/IPM. In some embodiments, a mass content of the crystallizable polymer or copolymer in the PC/IPM is at least 50 wt. %, at least 60 wt. %, at least 70 wt. %, at least 80 wt. %, at least 90 wt. %, at least 95 wt. %, at least 98 wt. %, or at least 99 wt. %. In some embodiments, a mass content of the crystallizable polymer or copolymer in the PC/IPM is in a range of 50 wt. % to 60 wt. %, 50 wt. % to 70 wt. %, 50 wt. % to 80 wt. %, 50 wt. % to 90 wt. %, 50 wt. % to 95 wt. %, 50 wt. % to 98 wt. %, 50 wt. % to 99 wt. %, 60 wt. % to 70 wt. %, 60 wt. % to 80 wt. %, 60 wt. % to 90 wt. %, 60 wt. % to 95 wt. %, 60 wt. % to 98 wt. %, 60 wt. % to 99 wt. %, 70 wt. % to 80 wt. %, 70 wt. % to 90 wt. %, 70 wt. % to 95 wt. %, 70 wt. % to 98 wt. %, 70 wt. % to 99 wt. %, 80 wt. % to 90 wt. %, 80 wt. % to 95 wt. %, 80 wt. % to 98 wt. %, 80 wt. % to 99 wt. %, 90 wt. % to 95 wt. %, 90 wt. % to 98 wt. %, 90 wt. % to 99 wt. %, or 95 wt. % to 99 wt. %.

In some embodiments, the PC/IPM comprises one or more catalysts (e.g., a catalyst used to control polymerization reactions). The presence of the one or more catalysts may help to control chain extension and/or branching reactions without addition of any additional catalysts. As an illustrative example, Example 22 shows that certain post-consumer PET flakes contained antimony and titanium, which are known as catalysts of transesterification and esterification reactions.

In some embodiments, the PC/IPM exhibits features characterized by pretreatment for subsequent enzymatic degradation. Such characteristics are determinable characteristics of the material itself, and would be clearly understood by those of ordinary skill in the art based on the descriptions herein as supplemented by knowledge available in the field. PC/IPMs characterized in this way are identifiable, determinable, and describable in ways that are not reliant upon or limited to any specific or formulaic process(es) of pretreatment which they have experienced. Instead, these characteristics are clear characteristics of the material itself. They can include some or all of the following, but need not include any specific characteristics if other characteristics would be indicators to those of ordinary skill in the art that the material exhibits features related to pretreatment: degree of crystallinity or semi-crystallinity, shear storage modulus (e.g., in a molten state), shear loss modulus (e.g., in a molten state), crystallization temperature and/or crystallization time, melt mass flow rate, etc.

In certain embodiments, the pretreatment comprises reacting a PC/IPM precursor comprising the crystallizable polymer or copolymer with a reactive agent. The reactive agent may be any reactive agent described herein, and the reacting may occur according to any method described herein.

In some embodiments, the PC/IPM has a relatively high linear shear complex modulus G*. In some embodiments, the PC/IPM has a linear shear complex modulus G* measured at a temperature 30° C. above the melting temperature of the crystallizable polymer or copolymer and at an angular frequency of 1.0 rad/s of at least 0.5 kPa, at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 200 kPa, at least 300 kPa, at least 400 kPa, at least 500 kPa, or at least 1 MPa. In some embodiments, the PC/IPM has a linear shear complex modulus G* measured at a temperature 30° C. above the melting temperature of the crystallizable polymer or copolymer and at an angular frequency of 1.0 rad/s in a range from 0.5 kPa to 1 kPa, 0.5 kPa to 5 kPa, 0.5 kPa to 10 kPa, 0.5 kPa to 15 kPa, 0.5 kPa to 20 kPa, 0.5 kPa to 50 kPa, 0.5 kPa to 100 kPa, 0.5 kPa to 200 kPa, 0.5 kPa to 500 kPa, 0.5 kPa to 1 MPa, 1 kPa to 5 kPa, 1 kPa to 10 kPa, 1 kPa to 15 kPa, 1 kPa to 20 kPa, 1 kPa to 50 kPa, 1 kPa to 100 kPa, 1 kPa to 200 kPa, 1 kPa to 500 kPa, 1 kPa to 1 MPa, 2 kPa to 5 kPa, 2 kPa to 10 kPa, 2 kPa to 15 kPa, 2 kPa to 20 kPa, 2 kPa to 50 kPa, 2 kPa to 100 kPa, 2 kPa to 500 kPa, 2 kPa to 1 MPa, 5 kPa to 10 kPa, 5 kPa to 15 kPa, 5 kPa to 20 kPa, 5 kPa to 50 kPa, 5 kPa to 100 kPa, 5 kPa to 500 kPa, 5 kPa to 1 MPa, 10 kPa to 20 kPa, 10 kPa to 50 kPa, 10 kPa to 100 kPa, 10 kPa to 500 kPa, 10 kPa to 1 MPa, 15 kPa to 50 kPa, 15 kPa to 100 kPa, 15 kPa to 500 kPa, 15 kPa to 1 MPa, 20 kPa to 50 kPa, 20 kPa to 100 kPa, 20 kPa to 500 kPa, 20 kPa to 1 MPa, 50 kPa to 100 kPa, 50 kPa to 500 kPa, 50 kPa to 1 MPa, 100 kPa to 500 kPa, 100 kPa to 1 MPa, or 500 kPa to 1 MPa.

In some embodiments, the PC/IPM has a relatively high shear storage modulus G′. In some embodiments, the PC/IPM has a shear storage modulus G′ measured at a temperature 30° C. above the melting temperature of the crystallizable polymer or copolymer and at an angular frequency of 1.0 rad/s of at least 0.5 kPa, at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 500 kPa, or at least 1 MPa. In some embodiments, the PC/IPM has a shear storage modulus G′ measured at a temperature 30° C. above a melting temperature Tₘ of the crystallizable polymer or copolymer and at an angular frequency of 1.0 rad/s in a range from 1 kPa to 5 kPa, 1 kPa to 10 kPa, 1 kPa to 15 kPa, 1 kPa to 20 kPa, 1 kPa to 50 kPa, 1 kPa to 100 kPa, 1 kPa to 500 kPa, 1 kPa to 1 MPa, 2 kPa to 5 kPa, 2 kPa to 10 kPa, 2 kPa to 15 kPa, 2 kPa to 20 kPa, 2 kPa to 50 kPa, 2 kPa to 100 kPa, 2 kPa to 500 kPa, 2 kPa to 1 MPa, 5 kPa to 10 kPa, 5 kPa to 15 kPa, 5 kPa to 20 kPa, 5 kPa to 50 kPa, 5 kPa to 100 kPa, 5 kPa to 500 kPa, 5 kPa to 1 MPa, 10 kPa to 20 kPa, 10 kPa to 50 kPa, 10 kPa to 100 kPa, 10 kPa to 500 kPa, 10 kPa to 1 MPa, 15 kPa to 50 kPa, 15 kPa to 100 kPa, 15 kPa to 500 kPa, 15 kPa to 1 MPa, 20 kPa to 50 kPa, 20 kPa to 100 kPa, 20 kPa to 500 kPa, 20 kPa to 1 MPa, 50 kPa to 100 kPa, 50 kPa to 500 kPa, 50 kPa to 1 MPa, 100 kPa to 500 kPa, 100 kPa to 1 MPa, or 500 kPa to 1 MPa. In some embodiments, the shear storage modulus G′ of at least 50%, at least 60%, at least 70%, at least 80%, at least 90%, at least 95%, at least 98%, at least 99%, or about 100% of the PC/IPM is in one or more of the above-listed ranges. In some embodiments, the shear storage modulus G′ of 50-70%, 50-80%, 50-90%, 50-95%, 50-98%, 50-100%, 60-80%, 60-90%, 60-95%, 60-98%, 60-99%, 60-100%, 70-90%, 70-95%, 70-98%, 70-99%, 70-100%, 80-90%, 80-95%, 80-98%, 80-99%, 80-100%, 90-95%, 90-98%, 90-99%, 90-100%, 95-98%, 95-99%, 95-100%, 98-100%, or 99-100% of the PC/IPM is in one or more of the above-listed ranges. The shear storage modulus G′ may be obtained using a rheometer (e.g., a TA Ares-G2 analyzer). In some cases, for example, the shear storage modulus G′ may be measured using the rheometer at a temperature 30° C. above a melting temperature Tₘ of the crystallizable polymer or copolymer, at 0.5% strain, and at an angular frequency of 1.0 rad/s.

In some embodiments, the PC/IPM has a relatively high shear loss modulus G″. In some embodiments, the PC/IPM has a shear loss modulus G″ measured at a temperature 30° C. above a melting temperature Tₘ of the crystallizable polymer or copolymer and an angular frequency of 1.0 rad/s of at least 0.5 kPa, at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 500 kPa, or at least 1 MPa. In some embodiments, the PC/IPM has a shear loss modulus G″ measured at a temperature 30° C. above a melting temperature Tₘ of the crystallizable polymer or copolymer and an angular frequency of 1.0 rad/s in a range from 1 kPa to 5 kPa, 1 kPa to 10 kPa, 1 kPa to 15 kPa, 1 kPa to 20 kPa, 1 kPa to 50 kPa, 1 kPa to 100 kPa, 1 kPa to 500 kPa, 1 kPa to 1 MPa, 2 kPa to 5 kPa, 2 kPa to 10 kPa, 2 kPa to 15 kPa, 2 kPa to 20 kPa, 2 kPa to 50 kPa, 2 kPa to 100 kPa, 2 kPa to 500 kPa, 2 kPa to 1 MPa, 5 kPa to 10 kPa, 5 kPa to 15 kPa, 5 kPa to 20 kPa, 5 kPa to 50 kPa, 5 kPa to 100 kPa, 5 kPa to 500 kPa, 5 kPa to 1 MPa, 10 kPa to 20 kPa, 10 kPa to 50 kPa, 10 kPa to 100 kPa, 10 kPa to 500 kPa, 10 kPa to 1 MPa, 15 kPa to 50 kPa, 15 kPa to 100 kPa, 15 kPa to 500 kPa, 15 kPa to 1 MPa, 20 kPa to 50 kPa, 20 kPa to 100 kPa, 20 kPa to 500 kPa, 20 kPa to 1 MPa, 50 kPa to 100 kPa, 50 kPa to 500 kPa, 50 kPa to 1 MPa, 100 kPa to 500 kPa, 100 kPa to 1 MPa, or 500 kPa to 1 MPa. In some embodiments, the shear loss modulus G″ of at least 50%, at least 60%, at least 70%, at least 80%, at least 90%, at least 95%, at least 98%, at least 99%, or about 100% of the PC/IPM is in one or more of the above-listed ranges. In some embodiments, the shear loss modulus G″ of 50-70%, 50-80%, 50-90%, 50-95%, 50-98%, 50-100%, 60-80%, 60-90%, 60-95%, 60-98%, 60-99%, 60-100%, 70-90%, 70-95%, 70-98%, 70-99%, 70-100%, 80-90%, 80-95%, 80-98%, 80-99%, 80-100%, 90-95%, 90-98%, 90-99%, 90-100%, 95-98%, 95-99%, 95-100%, 98-100%, or 99-100% of the PC/IPM is in one or more of the above-listed ranges. The shear loss modulus G″ may be obtained using a rheometer (e.g., a TA Ares-G2 analyzer). In some cases, for example, the shear loss modulus G″ may be measured using the rheometer at a temperature 30° C. above a melting temperature Tₘ of the crystallizable polymer or copolymer, at 0.5% strain, and at an angular frequency of 1.0 rad/s.

In some embodiments, the PC/IPM exhibiting features characterized by pretreatment for subsequent enzymatic degradation exhibits features differing from features of a comparative polymeric material. In some embodiments, the comparative polymeric material is the crystallizable polymer or copolymer in virgin form (i.e., crystallizable polymer or copolymer that has been produced directly from petrochemical feedstock, such as crude oil and/or natural gas, and has not been used or processed for use in a consumer product, industrial product, or industrial process). For example, if a PC/IPM comprises at least 50 wt. % PET, the comparative polymeric material may be virgin PET. In some embodiments, the comparative polymeric material is a polymeric material that is essentially identical to the PC/IPM except that it does not exhibit features characterized by the pretreatment for subsequent enzymatic degradation (e.g., it has not undergone a pretreatment as described herein). In certain embodiments, for example, the comparative polymeric material comprises a PC/IPM precursor that comprises the crystallizable polymer or copolymer and has not been reacted with the reactive agent. As an illustrative example, mixed plastic waste may be collected and may undergo mechanical and/or chemical processing (e.g., grinding, sorting, mixing, melting, homogenizing). A first portion of the collected and processed plastic waste may subsequently undergo pretreatment as described herein, and a second portion of the collected and processed plastic waste may remain untreated. In certain instances, the resulting first portion may constitute a PC/IPM and the second resulting portion may constitute a comparative polymeric material of the PC/IPM.

In some embodiments, the PC/IPM has different crystallization properties than the comparative polymeric material. In certain embodiments, for example, the PC/IPM has a longer crystallization time and/or a lower crystallization temperature than the comparative polymeric material.

In some embodiments, the PC/IPM has a longer crystallization time (e.g., the total length of time it takes to complete the crystallization process or the time at which the maximum heat flux is achieved in a DSC trace) than the comparative polymeric material at a given measurement temperature (e.g., a temperature 30° C. above the glass transition temperature of the crystallizable polymer or copolymer, a temperature 5° C. above the glass transition temperature of the crystallizable polymer or copolymer). In some cases, a longer crystallization time may advantageously delay and/or prevent crystallization during enzymatic degradation.

In certain embodiments, the PC/IPM (e.g., the PC/IPM fast cooled from a melt) has a crystallization time at a measurement temperature 30° C. above the glass transition temperature of the crystallizable polymer or copolymer that is at least 1.1 times, at least 2 times, at least 5 times, at least 8 times, or at least 10 times longer than a crystallization time of the comparative polymeric material at the same measurement temperature. In certain embodiments, the PC/IPM has a crystallization time at a measurement temperature 30° C. above the glass transition temperature of the crystallizable polymer or copolymer that is 1.1 to 2 times, 1.1 to 5 times, 1.1 to 8 times, 1.1 to 10 times, 2 to 5 times, 2 to 8 times, 2 to 10 times, 5 to 8 times, 5 to 10 times, or 8 to 10 times longer than a crystallization time of the comparative polymeric material at the same measurement temperature. In certain embodiments, the PC/IPM has a crystallization time measured at a measurement temperature 30° C. above the glass transition temperature of the crystallizable polymer or copolymer that is at least 3 minutes, at least 5 minutes, at least 10 minutes, at least 15 minutes, at least 30 minutes, at least 60 minutes, at least 120 minutes, at least 180 minutes, at least 240 minutes, at least 300 minutes, at least 360 minutes, or at least 420 minutes, at least 480 minutes, at least 540 minutes, or at least 600 minutes longer than a crystallization time of the comparative polymeric material measured at the same measurement temperature. In some embodiments, the PC/IPM has a crystallization time at a measurement temperature 30° C. above the glass transition temperature of the crystallizable polymer or copolymer that is longer than the crystallization time of the comparative polymeric material at the same measurement temperature by 3 to 5 minutes, 3 to 10 minutes, 3 to 15 minutes, 3 to 30 minutes, 3 to 60 minutes, 3 to 120 minutes, 3 to 180 minutes, 3 to 240 minutes, 3 to 300 minutes, 3 to 360 minutes, 3 to 420 minutes, 3 to 480 minutes, 3 to 540 minutes, 3 to 600 minutes, 5 to 10 minutes, 5 to 15 minutes, 5 to 30 minutes, 5 to 60 minutes, 5 to 120 minutes, 5 to 180 minutes, 5 to 240 minutes, 5 to 300 minutes, 5 to 360 minutes, 5 to 420 minutes, 5 to 480 minutes, 5 to 540 minutes, 5 to 600 minutes, 10 to 15 minutes, 10 to 30 minutes, 10 to 60 minutes, 10 to 120 minutes, 10 to 180 minutes, 10 to 240 minutes, 10 to 300 minutes, 10 to 360 minutes, 10 to 420 minutes, 10 to 480 minutes, 10 to 540 minutes, 10 to 600 minutes, 30 to 60 minutes, 30 to 120 minutes, 30 to 180 minutes, 30 to 240 minutes, 30 to 300 minutes, 30 to 360 minutes, 30 to 420 minutes, 30 to 480 minutes, 30 to 540 minutes, 30 to 600 minutes, 60 to 120 minutes, 60 to 180 minutes, 60 to 240 minutes, 60 to 300 minutes, 60 to 360 minutes, 60 to 420 minutes, 60 to 480 minutes, 60 to 540 minutes, 60 to 600 minutes, 120 to 180 minutes, 120 to 240 minutes, 120 to 300 minutes, 120 to 360 minutes, 120 to 420 minutes, 120 to 480 minutes, 120 to 540 minutes, 120 to 600 minutes, 180 to 240 minutes, 180 to 300 minutes, 180 to 360 minutes, 180 to 420 minutes, 180 to 480 minutes, 180 to 540 minutes, 180 to 600 minutes, 240 to 300 minutes, 240 to 360 minutes, 240 to 420 minutes, 240 to 480 minutes, 240 to 540 minutes, 240 to 600 minutes, 300 to 360 minutes, 300 to 420 minutes, 300 to 480 minutes, 300 to 540 minutes, 300 to 600 minutes, 360 to 420 minutes, 360 to 480 minutes, 360 to 540 minutes, 360 to 600 minutes, 420 to 480 minutes, 420 to 540 minutes, 420 to 600 minutes, 480 to 540 minutes, 480 to 600 minutes, or 540 to 600 minutes. The crystallization time may be measured using isothermal differential scanning calorimetry (DSC), with heat flow being monitored as a function of incubation time at the measurement temperature. Additional details regarding measurement of crystallization time are described with respect to Comparative Example 4 and Example 18.

In some embodiments, the PC/IPM has a lower crystallization temperature when cooled from a melt (e.g., at a rate of 20° C./min) than the comparative polymeric material. In some cases, a lower crystallization temperature may advantageously delay and/or prevent crystallization during enzymatic degradation. In some embodiments, the PC/IPM has a crystallization temperature when cooled from a melt (e.g., at a rate of 20° C./min) that is at least 1° C., at least 2° C., at least 3° C., at least 4° C., at least 5° C., at least 6° C., at least 7° C., at least 8° C., at least 9° C., at least 10° C., at least 15° C., or at least 20° C. lower than a crystallization temperature of the comparative polymeric material when cooled from a melt (e.g., at a rate of 20° C./min). In some embodiments, the PC/IPM has a crystallization temperature when cooled from melt (e.g., at a rate of 20° C./min) that is in a range from 1° C. to 5° C., 1° C. to 10° C., 1° C. to 15° C., 1° C. to 20° C., 5° C. to 10° C., 5° C. to 15° C., 5° C. to 20° C., 10° C. to 15° C., 10° C. to 20° C., or 15° C. to 20° C. lower than a crystallization temperature of the comparative polymeric material when cooled from a melt (e.g., at a rate of 20° C./min). The crystallization temperature may be measured using differential scanning calorimetry (DSC). For example, DSC heating scans may be obtained using a calorimeter (e.g., a TA Discovery Q200 calorimeter). A sample comprising the PC/IPM or the comparative polymeric material may be heated from 0° C. to 300° C. at a heating rate of 10° C./min, and the crystallization temperature may be obtained from the resulting normalized heat flow v. temperature curve. Additional details regarding measurement of crystallization temperature are described with respect to Example 9.

In some embodiments, the PC/IPM has a lower heat of crystallization when cooled from a melt (e.g., at a rate of 20° C./min) than the comparative polymeric material. In some embodiments, a heat of crystallization of the PC/IPM when cooled from a melt (e.g., at a rate of 20° C./min) is at least 5%, at least 10%, at least 15%, at least 20%, at least 30%, at least 40%, or at least 50% lower than a heat of crystallization of the comparative polymeric material when cooled from a melt (e.g., at a rate of 20° C./min). In some embodiments, a heat of crystallization of the PC/IPM when cooled from a melt (e.g., at a rate of 20° C./min) is 5 to 10%, 5 to 15%, 5 to 20%, 5 to 30%, 5 to 40%, 5 to 50%, 10 to 15%, 10 to 20%, 10 to 30%, 10 to 40%, 10 to 50%, 15 to 20%, 15 to 30%, 15 to 40%, 15 to 50%, 20 to 30%, 20 to 40%, 20 to 50%, 30 to 40%, 30 to 50%, or 40 to 50% lower than a heat of crystallization of the comparative polymeric material when cooled from a melt (e.g., at a rate of 20° C./min). In some embodiments, the heat of crystallization may be measured using DSC. For example, a sample may be heated from 0° C. to 300° C. at a heating rate of 10° C./min in a calorimeter (e.g., a TA Discovery Q200 calorimeter), and the heat of crystallization may be obtained from the resulting normalized heat flow v. temperature curve.

In some embodiments, the PC/IPM has a lower melt mass-flow rate (MFR) than the comparative polymeric material. The melt mass-flow rate generally refers to the case of flow of a melted material. In some cases, a relatively low melt mass-flow rate may be indicative of increased crosslinking, branching, and/or extension. In some embodiments, the PC/IPM has a melt mass-flow rate measured at a given measurement temperature (e.g., 30° C. above the melting temperature of the crystallizable polymer or copolymer) that is at least 3 times lower, at least 5 times lower, at least 8 times lower, at least 10 times lower, at least 15 times lower, or at least 20 times lower than a mass melt-flow rate of the comparative polymeric material at the given measurement temperature. In some embodiments, a melt mass-flow rate of the PC/IPM measured at a given measurement temperature (e.g., 30° C. above the melting temperature of the crystallizable polymer or copolymer) is 3 to 5 times lower, 3 to 10 times lower, 3 to 15 times lower, 3 to 20 times lower, 5 to 10 times lower, 5 to 15 times lower, 5 to 20 times lower, 10 to 15 times lower, 10 to 20 times lower, or 15 to 20 times lower than a mass melt-flow rate of the comparative polymeric material at the given measurement temperature.

In some embodiments, the PC/IPM does not flow. In certain instances, for example, a PC/IPM that has undergone annealing may not flow.

In some embodiments, the PC/IPM has a higher linear shear complex modulus G* than the comparative polymeric material. In some embodiments, a linear shear complex modulus G* of the PC/IPM measured at a temperature 30° C. above the melting temperature of the crystallizable polymer or copolymer and at an angular frequency of 1.0 rad/s is higher than a linear shear complex modulus G* of the comparative polymeric material measured under the same conditions. In certain embodiments, a linear shear complex modulus G* of the PC/IPM measured at a temperature 30° C. above the melting temperature of the crystallizable polymer or copolymer and at an angular frequency of 1.0 rad/s is at least 40, at least 50, at least 80, at least 100, at least 200, at least 300, at least 400, at least 500, at least 600, at least 700, at least 800, at least 900, at least 1,000, at least 2,000, at least 5,000, at least 8,000, at least 10,000, at least 15,000, at least 20,000, or at least 22,000 times higher than a linear shear complex modulus G* of the comparative polymeric material measured under the same conditions. In certain embodiments, a linear shear complex modulus G* of the PC/IPM measured at a temperature 30° C. above the melting temperature of the crystallizable polymer or copolymer and at an angular frequency of 1.0 rad/s is in a range from 40 to 100 times higher, 40 to 200 times higher, 40 to 500 times higher, 40 to 1,000 times higher, 40 to 2,000 times higher, 40 to 5,000 times higher, 40 to 10,000 times higher, 40 to 15,000 times higher, 40 to 20,000 times higher, 40 to 22,000 times higher, 50 to 100 times higher, 50 to 200 times higher, 50 to 500 times higher, 50 to 1,000 times higher, 50 to 2,000 times higher, 50 to 5,000 times higher, 50 to 10,000 times higher, 50 to 15,000 times higher, 50 to 20,000 times higher, 50 to 22,000 times higher, 100 to 200 times higher, 100 to 500 times higher, 100 to 1,000 times higher, 100 to 2,000 times higher, 100 to 5,000 times higher, 100 to 10,000 times higher, 100 to 15,000 times higher, 100 to 20,000 times higher, 100 to 22,000 times higher, 200 to 500 times higher, 200 to 1,000 times higher, 200 to 2,000 times higher, 200 to 5,000 times higher, 200 to 10,000 times higher, 200 to 15,000 times higher, 200 to 20,000 times higher, 200 to 22,000 times higher, 500 to 1,000 times higher, 500 to 2,000 times higher, 500 to 5,000 times higher, 500 to 10,000 times higher, 500 to 15,000 times higher, 500 to 20,000 times higher, 500 to 22,000 times higher, 1,000 to 5,000 times higher, 1,000 to 10,000 times higher, 1,000 to 15,000 times higher, 1,000 to 20,000 times higher, 1,000 to 22,000 times higher, 5,000 to 10,000 times higher, 5,000 to 15,000 times higher, 5,000 to 20,000 times higher, 5,000 to 22,000 times higher, 10,000 to 15,000 times higher, 10,000 to 20,000 times higher, 10,000 to 22,000 times higher, 15,000 to 20,000 times higher, 15,000 to 22,000 times higher, or 20,000 to 22,000 times higher than a linear shear complex modulus G* of the comparative polymeric material measured under the same conditions.

In some embodiments, the PC/IPM has a higher shear storage modulus G′ than the comparative polymeric material. In some embodiments, a shear storage modulus G′ of the PC/IPM measured at a temperature 30° C. above the melting temperature of the crystallizable polymer or copolymer and an angular frequency of 0.1 rad/s is higher than a shear storage modulus G′ of the comparative polymeric material measured under the same conditions. In certain embodiments, a shear storage modulus G′ of the PC/IPM measured at a temperature 30° C. above the melting temperature of the crystallizable polymer or copolymer and an angular frequency of 1.0 rad/s is at least 50, at least 100, at least 200, at least 300, at least 400, at least 500, at least 600, at least 700, at least 800, at least 900, at least 1,000, at least 2,000, at least 5,000, at least 10,000, at least 15,000, at least 20,000, or at least 22,000 times higher than a shear storage modulus G′ of the comparative polymeric material measured under the same conditions. In certain embodiments, a shear storage modulus G′ of the PC/IPM measured at a temperature 30° C. above the melting temperature of the crystallizable polymer or copolymer and an angular frequency of 1.0 rad/s is in a range from 50 to 100 times higher, 50 to 200 times higher, 50 to 500 times higher, 50 to 1,000 times higher, 50 to 2,000 times higher, 50 to 5,000 times higher, 50 to 10,000 times higher, 50 to 15,000 times higher, 50 to 20,000 times higher, 50 to 22,000 times higher, 100 to 200 times higher, 100 to 500 times higher, 100 to 1,000 times higher, 100 to 2,000 times higher, 100 to 5,000 times higher, 100 to 10,000 times higher, 100 to 15,000 times higher, 100 to 20,000 times higher, 100 to 22,000 times higher, 200 to 500 times higher, 200 to 1,000 times higher, 200 to 2,000 times higher, 200 to 5,000 times higher, 200 to 10,000 times higher, 200 to 15,000 times higher, 200 to 20,000 times higher, 200 to 22,000 times higher, 500 to 1,000 times higher, 500 to 2,000 times higher, 500 to 5,000 times higher, 500 to 10,000 times higher, 500 to 15,000 times higher, 500 to 20,000 times higher, 500 to 22,000 times higher, 1,000 to 5,000 times higher, 1,000 to 10,000 times higher, 1,000 to 15,000 times higher, 1,000 to 20,000 times higher, 1,000 to 22,000 times higher, 5,000 to 10,000 times higher, 5,000 to 15,000 times higher, 5,000 to 20,000 times higher, 5,000 to 22,000 times higher, 10,000 to 15,000 times higher, 10,000 to 20,000 times higher, 10,000 to 22,000 times higher, 15,000 to 20,000 times higher, 15,000 to 22,000 times higher, or 20,000 to 22,000 times higher than a shear storage modulus G′ of the comparative polymeric material measured under the same conditions.

In some embodiments, the PC/IPM has a higher shear loss modulus G″ than the comparative polymeric material. In some embodiments, a shear loss modulus G″ of the PC/IPM measured at a temperature 30° C. above the melting temperature of the crystallizable polymer or copolymer and an angular frequency of 1.0 rad/s is higher than a shear loss modulus G″ of the comparative polymeric material measured under the same conditions. In certain embodiments, a shear loss modulus G″ of the PC/IPM measured at a temperature 30° C. above the melting temperature of the crystallizable polymer or copolymer and an angular frequency of 1.0 rad/s is at least 50, at least 100, at least 200, at least 300, at least 400, at least 500, at least 600, at least 700, at least 800, at least 900, at least 1,000, at least 2,000, at least 5,000, at least 10,000, or at least 16,000 times higher than a shear loss modulus G″ of the comparative polymeric material measured under the same conditions. In certain embodiments, a shear loss modulus G″ of the PC/IPM measured at a temperature 30° C. above the melting temperature of the crystallizable polymer or copolymer and an angular frequency of 1.0 rad/s is in a range from 50 to 100 times higher, 50 to 200 times higher, 50 to 500 times higher, 50 to 1,000 times higher, 50 to 2,000 times higher, 50 to 5,000 times higher, 50 to 10,000 times higher, 50 to 16,000 times higher, 100 to 200 times higher, 100 to 500 times higher, 100 to 1,000 times higher, 100 to 2,000 times higher, 100 to 5,000 times higher, 100 to 10,000 times higher, 100 to 16,000 times higher, 200 to 500 times higher, 200 to 1,000 times higher, 200 to 2,000 times higher, 200 to 5,000 times higher, 200 to 10,000 times higher, 200 to 16,000 times higher, 500 to 1,000 times higher, 500 to 2,000 times higher, 500 to 5,000 times higher, 500 to 10,000 times higher, 500 to 16,000 times higher, 1,000 to 5,000 times higher, 1,000 to 10,000 times higher, 1,000 to 16,000 times higher, 5,000 to 10,000 times higher, 5,000 to 16,000 times higher, or 10,000 to 16,000 times higher than a shear loss modulus G″ of the comparative polymeric material measured under the same conditions.

In some embodiments, a PC/IPM (e.g., the crystallizable polymer or copolymer chains of the PC/IPM) has a higher weight average molecular weight than the comparative polymeric material. In certain embodiments, the PC/IPM has a weight average molecular weight that is at least 5% higher, at least 10% higher, at least 20% higher, at least 50% higher, or at least 80% higher than a weight average molecular weight of the comparative polymeric material. In certain embodiments, the PC/IPM has a weight average molecular weight that is 5% to 10% higher, 5% to 20% higher, 5% to 50% higher, 5% to 80% higher, 10% to 20% higher, 10% to 50% higher, 10% to 80% higher, 20% to 50% higher, 20% to 80% higher, or 50% to 80% higher than a weight average molecular weight of the comparative polymeric material. The weight average molecular weight of the PC/IPM and/or the comparative polymeric material may be measured by size exclusion chromatography, dynamic light scattering, and/or rheology in a melt.

In some embodiments, a PC/IPM has a higher intrinsic viscosity than the comparative polymeric material. In certain embodiments, the PC/IPM has an intrinsic viscosity that is at least 5% higher, at least 10% higher, at least 20% higher, at least 50% higher, or at least 80% higher than an intrinsic viscosity of the comparative polymeric material. In certain embodiments, the PC/IPM has an intrinsic viscosity that is 5% to 10% higher, 5% to 20% higher, 5% to 50% higher, 5% to 80% higher, 10% to 20% higher, 10% to 50% higher, 10% to 80% higher, 20% to 50% higher, 20% to 80% higher, or 50% to 80% higher than an intrinsic viscosity of the comparative polymeric material.

In some embodiments, the PC/IPM has a higher gel content than the comparative polymeric material. In some embodiments, the gel content of the PC/IPM is at least 1%, at least 2%, at least 5%, at least 10%, at least 20%, or at least 50% higher than the gel content of the comparative polymeric material. In certain embodiments, the PC/IPM has a gel content that is 1% to 2% higher, 1% to 5% higher, 1% to 10% higher, 1% to 20% higher, 1% to 50% higher, 2% to 5% higher, 2% to 10% higher, 2% to 20% higher, 2% to 50% higher, 5% to 10% higher, 5% to 20% higher, 5% to 50% higher, 10% to 20% higher, 10% to 50% higher, or 20% to 50% higher than a gel content of the comparative polymeric material. Gel content of a material may be measured by separating a soluble fraction and an insoluble fraction of the material (e.g., by long dissolution followed by filtration or by using a Soxhlet), with gel content corresponding to the dry weight fraction.

In some embodiments, the PC/IPM comprises a plurality of particles. In some cases, the plurality of PC/IPM particles comprises relatively large particles. In some cases, it may be possible for polymer-degrading enzymes to degrade the PC/IPM at a higher rate than the comparative polymeric material. Polymer-degrading enzymes may therefore be able to degrade larger particles of the PC/IPM than of the comparative polymeric material. In certain cases, this ability to enzymatically degrade larger particles of the pretreated polymeric material may advantageously reduce the need to achieve smaller particle sizes by milling and/or sorting particles of the PC/IPM.

In some embodiments, the plurality of PC/IPM particles has an average particle size of 5 mm or less, 4 mm or less, 3 mm or less, 2 mm or less, 1 mm or less, 600 μm or less, 500 μm or less, 400 μm or less, 300 μm or less, 200 μm or less, 100 μm or less, 50 μm or less, or 25 μm or less. In some embodiments, the plurality of PC/IPM particles has an average particle size in a range from 25 μm to 50 μm, 25 μm to 100 μm, 25 μm to 200 μm, 25 μm to 300 μm, 25 μm to 400 μm, 25 μm to 500 μm, 25 μm to 600 μm, 25 μm to 1 mm, 25 μm to 2 mm, 25 μm to 3 mm, 25 μm to 4 mm, 25 μm to 5 mm, 50 μm to 100 μm, 50 μm to 200 μm, 50 μm to 300 μm, 50 μm to 400 μm, 50 μm to 500 μm, 50 μm to 600 μm, 50 μm to 1 mm, 50 μm to 2 mm, 50 μm to 3 mm, 50 μm to 4 mm, 50 μm to 5 mm, 100 μm to 200 μm, 100 μm to 300 μm, 100 μm to 400 μm, 100 μm to 500 μm, 100 μm to 600 μm, 100 μm to 1 mm, 100 μm to 2 mm, 100 μm to 3 mm, 100 μm to 4 mm, 100 μm to 5 mm, 200 μm to 300 μm, 200 μm to 400 μm, 200 μm to 500 μm, 200 μm to 600 μm, 200 μm to 1 mm, 200 μm to 2 mm, 200 μm to 3 mm, 200 μm to 4 mm, 200 μm to 5 mm, 300 μm to 400 μm, 300 μm to 500 μm, 300 μm to 600 μm, 300 μm to 1 mm, 300 μm to 2 mm, 300 μm to 3 mm, 300 μm to 4 mm, 300 μm to 5 mm, 400 μm to 500 μm, 400 μm to 600 μm, 400 μm to 1 mm, 400 μm to 2 mm, 400 μm to 3 mm, 400 μm to 4 mm, 400 μm to 5 mm, 500 μm to 600 μm, 500 μm to 1 mm, 500 μm to 2 mm, 500 μm to 3 mm, 500 μm to 4 mm, 500 μm to 5 mm, 600 μm to 1 mm, 600 μm to 2 mm, 600 μm to 3 mm, 600 μm to 4 mm, 600 μm to 5 mm, 1 mm to 2 mm, 1 mm to 3 mm, 1 mm to 4 mm, 1 mm to 5 mm, 2 mm to 4 mm, 2 mm to 5 mm, 3 mm to 5 mm, or 4 mm to 5 mm. As used herein, the “size” of a particle refers to the maximum distance between two opposed boundaries of an individual particle that can be measured (e.g., a diameter, a length). The “average size” of a plurality of particles refers to the number average of the size of the particles. The average particle size may be determined according to any method known in the art, such as laser diffraction and/or dynamic image analysis.

In some embodiments, the plurality of PC/IPM particles has a relatively broad particle size distribution. As noted above, polymer-degrading enzymes may be able to degrade larger particles of the PC/IPM than the comparative polymeric material and, therefore, may be able to degrade particles having a broader size distribution than would otherwise be possible without pretreatment. In some embodiments, the standard deviation of particle sizes of the plurality of PC/IPM particles is at least 10%, 20%, 30%, 40%, or 50% of the average particle size. In some embodiments, the standard deviation of particle sizes of the plurality of PC/IPM particles is in a range from 10% to 20%, 10% to 30%, 10% to 40%, 10% to 50%, 20% to 30%, 20% to 40%, 20% to 50%, 30% to 40%, 30% to 50%, or 40% to 50% of the average particle size. Standard deviation (o) is given its normal meaning in the art and can be calculated according to Equation 2. The percentage comparisons between the standard deviation and the average particle size outlined above can be obtained by dividing the standard deviation by the average particle size and multiplying by 100%.

Some aspects are directed to a material configured for enzymatic degradation. In some embodiments, the material comprises a post-consumer and/or post-industrial polymeric material (PC/IPM) exhibiting features characterized by a pretreatment for subsequent enzymatic degradation.

In certain embodiments, the PC/IPM comprises at least 50 wt. %, at least 60 wt. %, at least 70 wt. %, at least 80 wt. %, at least 90 wt. %, at least 95 wt. %, at least 98 wt. %, or at least 99 wt. % of polyethylene terephthalate (PET). In some embodiments, a mass content of PET in the PC/IPM is in a range of 50 wt. % to 60 wt. %, 50 wt. % to 70 wt. %, 50 wt. % to 80 wt. %, 50 wt. % to 90 wt. %, 50 wt. % to 95 wt. %, 50 wt. % to 98 wt. %, 50 wt. % to 99 wt. %, 60 wt. % to 70 wt. %, 60 wt. % to 80 wt. %, 60 wt. % to 90 wt. %, 60 wt. % to 95 wt. %, 60 wt. % to 98 wt. %, 60 wt. % to 99 wt. %, 70 wt. % to 80 wt. %, 70 wt. % to 90 wt. %, 70 wt. % to 95 wt. %, 70 wt. % to 98 wt. %, 70 wt. % to 99 wt. %, 80 wt. % to 90 wt. %, 80 wt. % to 95 wt. %, 80 wt. % to 98 wt. %, 80 wt. % to 99 wt. %, 90 wt. % to 95 wt. %, 90 wt. % to 98 wt. %, 90 wt. % to 99 wt. %, or 95 wt. % to 99 wt. %.

In certain embodiments, the PC/IPM has a crystallization temperature less than 199° C. when cooled from a melt at a rate of 20° C./min. In certain embodiments, the PC/IPM has a crystallization time of at least 16 minutes when measured at a temperature 30° C. above a glass transition temperature of PET after fast cooling from the melt. In certain embodiments, the PC/IPM has a heat of crystallization less than 48.5 J/g when cooled from the melt at a rate of 20° C./min.

In certain embodiments, the PC/IPM has a linear shear complex modulus G* of at least 1 kPa, at least 2 kPa, at least 3 kPa, at least 4 kPa, at least 5 kPa, at least 10 kPa, at least 15 kPa, at least 20 kPa, at least 25 kPa, at least 30 kPa, at least 40 kPa, at least 50 kPa, at least 60 kPa, at least 70 kPa, at least 80 kPa, at least 90 kPa, at least 100 kPa, at least 200 kPa, at least 300 kPa, at least 400 kPa, at least 500 kPa, or at least 1 MPa when measured at a temperature 30° C. above the melting temperature of PET and at an angular frequency of 1.0 rad/s.

In certain embodiments, the PC/IPM has a gel content of at least 10%.

Some aspects are directed to a polymeric material. In some embodiments, the polymeric material comprises a pretreated polymeric material produced by reacting polyethylene terephthalate (PET) with diglycidyl terephthalate (DGT). The reacting may occur according to any method described herein. In certain embodiments, a crystallization time of the pretreated polymeric material soaked at 70° C. in phosphate buffer at a given measurement temperature is at least 2 times longer than a crystallization time of polyethylene terephthalate at the given measurement temperature. In some instances, the given measurement temperature is a temperature 30° C. above a glass transition temperature of polyethylene terephthalate. The following examples are intended to illustrate certain embodiments of the present invention, but do not exemplify the full scope of the invention.

### EXAMPLES

### Example 1

### Reactive Mixing/Extrusion of PET and 5 wt. % DGT

PET pellets (50 g) (RAMAPET N1(S), Indorama Ventures having 322 ppm of antimony catalyst) were placed in a beaker, and the beaker was immersed into liquid nitrogen for 1 minute. Then the cooled PET pellets were transferred to a Moulinex grinder (50 g, 180 W) and milled for 1 minute. The obtained milled fraction was transferred to the beaker and cooled for 1 minute by immersing it in liquid nitrogen. Then the cooled powder was transferred to the Moulinex grinder and milled for 1 minute. Milled PET was dried at 150° C. for 6 hours in an oven under vacuum (Salvis Lab). Dried PET was mixed with reactive agent DGT (Denacol EX-711 Nagase ChemteX Corporation, 5 wt. %) and antioxidant (Irganox 1010, 0.1 wt. %) using a Moulinex grinder for 10 seconds. The obtained powder (14 g) was fed into a conical twin screw extruder (DSM, Xplore, 15 cm³ capacity) equipped with a co-rotating conical screw profile, a recirculation channel to control the residence time, and a circle die with a diameter of 3.0 mm. The extrusion was performed under circulation of nitrogen, with a barrel temperature profile as follows. Three temperature controls at different positions of the barrel were set as follows: top position (270° C.), middle position (270° C.), and exit position (280° C.). The speed of rotation of the screws was 60 RPM. The powder was fed in around 1.5 minutes and the residence time was defined as the time at which the axial force reached 7000 N. The axial force was kept under 7000 N to avoid blocking the extruder. The material was extruded directly into an ice water bath (5° C.) to be fast cooled in said bath.

### Example 2

### Reactive Mixing/Extrusion of PET and 0.75 wt. % DGT

A pretreated PET was prepared and synthesized under the same conditions as described in Example 1 with the only exception that the DGT composition was 0.75 wt. % and the material was extruded up to an axial force of 3000 N.

This Example, along with Examples 3 and 4, illustrates that it is possible to change the reactive agent composition during synthesis of pretreated PET without compromising capability of using reactive mixing or extrusion.

### Example 3

### Reactive Mixing/Extrusion of PET and 1 wt. % DGT

A pretreated PET was prepared and synthesized under the same conditions as described in Example 1 with the only exception that the DGT composition was 1 wt. % and the material was extruded up to an axial force of 3300 N.

### Example 4

### Reactive Mixing/Extrusion of PET and 3 wt. % DGT

A pretreated PET was prepared and synthesized under the same conditions as described in Example 1 with the only exception that the DGT composition was 3 wt. %.

### Example 5

Pretreated PET Synthesized by Reactive Extrusion/Mixing with 5 wt. % DGT Followed by Isothermal Annealing

A pretreated PET was prepared and synthesized under the same conditions as described in Example 1 with the only exception that after reactive extrusion or reactive mixing and fast cooling, the obtained extrudate was annealed at 280° C. for 10 minutes under vacuum using a hot plate vacuum desiccator. After annealing, the resulting material was fast cooled in a water bath at room temperature.

### Comparative Example 1

### PET by Extrusion and Fast Cooling

The following example describes the preparation of PET through standard extrusion followed by fast cooling in the absence of a reactive agent.

Indorama pellets were dried at 150° C. for 6 hours in an oven under vacuum (Salvis Lab). Dried pellets (14 g) were fed together with an antioxidant (Irganox 1010, 0.1 wt. %) into a conical twin screw extruder (DSM. Xplore, 15 cm³ capacity) equipped with a co-rotating conical screw profile, a recirculation channel to control the residence time, and a circle die with a diameter of 3.0 mm. The extrusion was performed under circulation of nitrogen, with a barrel temperature profile as follows. Three temperature controls at different positions of the barrel were set as follows: top position (270° C.), middle position (270° C.) and exit position (280° C.). The speed of rotation of the screws was 60 RPM. After PET feed (1.5 min) and a residence time of 2 minutes, the material was extruded directly into an ice water bath (5° C.) to be fast cooled in said bath.

### Comparative Example 2

### PET by Extrusion and Fast Cooling (Longer Residence Time)

A PET extrudate was prepared under the same conditions as described in Comparative Example 1 with the only exception that the residence time was increased from 2 minutes to 10 minutes.

### Example 6

Pretreated PET by Reactive Extrusion/Mixing with DGT 1 wt. % with Addition of Catalyst

A pretreated PET was prepared and synthesized under the same conditions as described in Example 3 with the only exception that the reactive extrusion or reactive mixing was performed in the presence of 0.1 wt. % of zinc acetylacetonate as a catalyst. The material was extruded up to an axial force of 8000 N.

### Example 7

Reactive Mixing/Extrusion of rPET and 5 wt. % DGT

100 g of post-consumer PET flakes (rPET) (PolyQuest, https://www.polyquest.com/products/pet-and-rpet/) were dried at 150° C. for 6 hours in an oven under vacuum (Salvis Lab). Dried rPET flakes were mixed with reactive agent DGT (Denacol EX-711 Nagase ChemteX Corporation, 5 wt. %) and antioxidant (Irganox 1010, 0.1 wt. %). The resulting sample (14 g) was fed into a conical twin screw extruder (DSM, Xplore, 15 cm³ capacity) equipped with a co-rotating conical screw profile, a recirculation channel to control the residence time, and a circle die with a diameter of 3.0 mm. The extrusion was performed under circulation of nitrogen, with a barrel temperature profile as follows. Three temperature controls at different positions of the barrel were set as follows: top position (270° C.), middle position (270° C.), and exit position (280° C.). The speed of rotation of the screws was 60 RPM. The flakes with DGT and antioxidant were fed in around 1.5 minutes and the residence time was defined as the time at which the axial force reached 7000 N. The axial force was kept under 7000 N to avoid blocking the extruder. The material was extruded directly into an ice water bath (5° C.) to be fast cooled in said bath.

### Example 8

Pretreated rPET Synthesized by Reactive Extrusion/Mixing with DGT 5 wt. % Followed by Isothermal Annealing

A pretreated rPET was prepared and synthesized under the same conditions as described in Example 7 with the only exception that after reactive extrusion or reactive mixing and fast cooling, the obtained extrudate was placed in a 300 μm thick stainless steel mold and was annealed at 280° C. for 20 minutes under vacuum using a hot plate vacuum desiccator. After the thermal annealing, the resulting material was fast cooled in a water bath at room temperature.

### Comparative Example 3

rPET by Extrusion and Fast Cooling

The following example describes the preparation of rPET following standard extrusion followed by fast cooling in the absence of a reactive agent.

rPET flakes were dried at 150° C. for 6 hours in an oven under vacuum (Salvis Lab). Dried flakes (14 g) were fed together with an antioxidant (Irganox 1010, 0.1 wt. %) into a conical twin screw extruder (DSM, Xplore, 15 cm³ capacity) equipped with a co-rotating conical screw profile, a recirculation channel to control the residence time, and a circle die with a diameter of 3.0 mm. The extrusion was performed under circulation of nitrogen, with a barrel temperature profile as follows. Three temperature controls at different positions of the barrel were set as follows: top position (270° C.), middle position (270° C.) and exit position (280° C.). The speed of rotation of the screws was 60 RPM. After rPET feed (1.5 min) and a residence time of 2 minutes, the material was extruded directly into an ice water bath (5° C.) to be fast cooled in said bath.

### Example 9

In this Example, the properties of the pretreated PET and rPET synthesized in Examples 1-8 and Comparative Examples 1-3 were characterized.

### Axial Force Curves

The axial force on the barrel of the compounder is a qualitative indicator of the change of viscosity during the reactive extrusion or reactive mixing, and it was monitored as a function of the reactive extrusion or reactive mixing time.

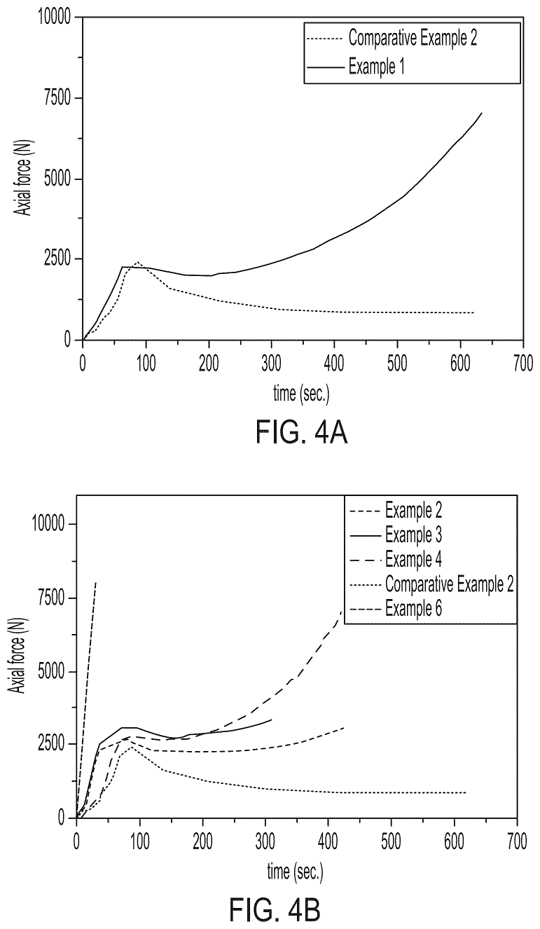

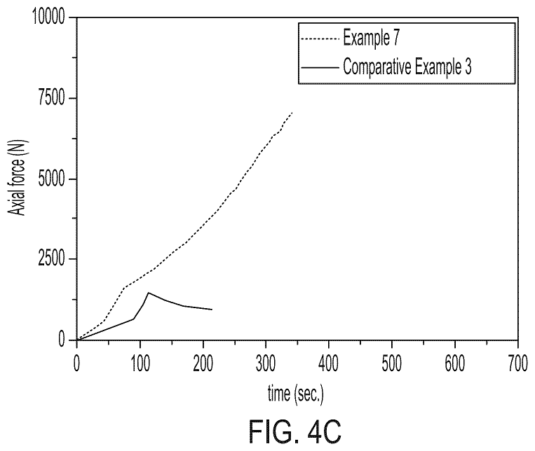

FIG. 4A shows the variation of the axial force as a function of the reactive extrusion or reactive mixing time for the conditions of Example 1 and Comparative Example 2 (a reference sample without DGT reactive agent). After the feeding time (1.5 min), it was observed that in the presence of DGT, the axial force remained almost constant for 2 minutes and then increased up to a value of 7000 N. The increase in the axial force is an indication of chain extension/branching/cross-linking. On the contrary, in the absence of the reactive agent (DGT), the axial force did not increase even after a residence time of 10 minutes (Comparative Example 2).

The increase of the axial force during reactive extrusion or reactive mixing was also observed for different (lower) contents of reactive agent as presented in FIG. 4B for the conditions described in Example 2, Example 3, Example 4, and Example 6. The curve corresponding to the absence of DGT (Comparative Example 2) is also included in FIG. 4B.

The increase of the axial force during reactive extrusion or reactive mixing was also observed for the conditions described in Example 7 in the presence of the reactive agent DGT, as presented in FIG. 4C. The curve corresponding to rPET in the absence of DGT (Comparative Example 3) is also included in FIG. 4C.

### Solubility Tests

Solubility tests were performed as follows: 50 mg of sample were immersed in 5 mL of Hexafluoro-2-propanol (HFIp, Sigma-Aldrich), a solvent for PET, and stirred under magnetic stirring at room temperature for 48 hours. The samples were classified as “soluble” if no residual material was observed by visual inspection after 48 hours of continuous stirring. The samples were classified as “insoluble” if after 48 hours of continuous stirring in HFIp, residues could be observed by visual inspection. The results are summarized in Table 2.

TABLE 2
Solubility of different PET samples in HFIp.

| Condition | Result |
|---|---|
| Example 1 | Soluble |
| Example 2 | Soluble |
| Example 3 | Soluble |
| Example 4 | Soluble |
| Example 5 | Insoluble |
| Comparative Example 1 | Soluble |
| Example 7 | Soluble |
| Example 8 | Insoluble |
| Comparative Example 3 | Soluble |

### Differential Scanning Calorimetry (DSC)

### DSC Heating Scans

The crystallinity degree determined from DSC first heating scan (CD) is defined by the following expression:

$$CD = \frac{( Δ {H}_{melt} - Δ {H}_{c r y s t a l l i z a t i o n}}{Δ {H}_{m e l t}^{°}} \cdot 100 \qquad ( 1 )$$

where ΔHₘₑₗₜ is the normalized enthalpy of melting of PET, ΔH_{crystallization} is the normalized enthalpy of crystallization of PET, and ΔHₘₑₗₜ° is the normalized enthalpy of melting of a 100% crystalline polymer and/or plastic waste at the melting temperature. Δₘₑₗₜ° for PET is 140.1 J/g.

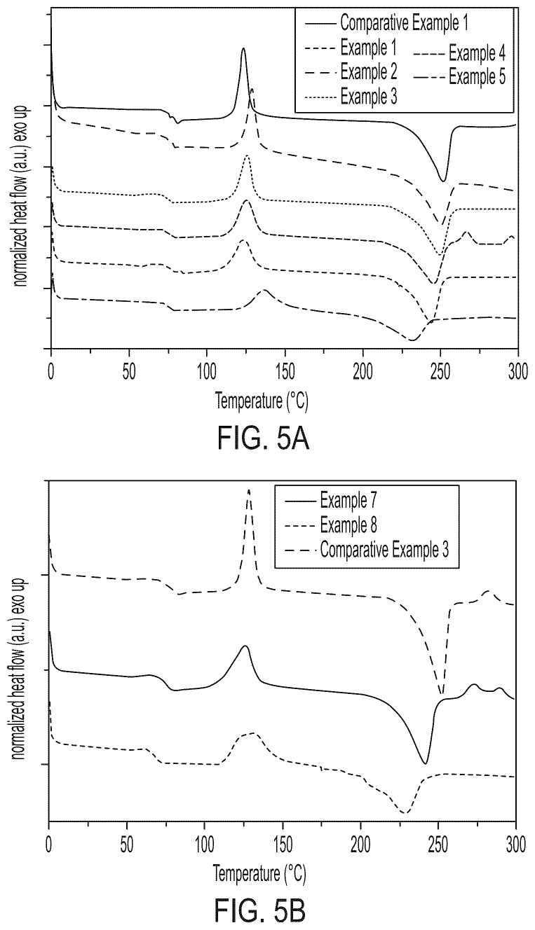

DSC first heating scans of PET samples were obtained using a calorimeter (TA, discovery Q200). 10 mg of PET extrudate sample were cut and introduced in a capsule (TA, Tzero Pan T 220228 and Tzero Hermetic Lid T 220315). The first heating scan was collected following the sequence: 1) Equilibrate temperature from room temperature to 0° C.; 2) Isothermal step (0° C.) for 1 min; 3) Heating step from 0° C. to 300° C. at a heating rate of 10° C./min. From the normalized heat flow curve v. temperature, the CD was calculated as defined in Equation 1 using the TRIOS software version v3.1.5.3696. The integration of the crystallization exothermic peak at around 110-140° C. and the melting endothermic peak at around 230-250° C. was performed from visually determined respective starting points to end points using a straight baseline between them to get ΔHₘₑₗₜ and ΔH_{crystallization}. The glass transition temperature (T_{g}) (midpoint), crystallization temperature (T_{crystallization}) (mean and onset) and melting temperature (T_{melting}) (mean and onset) were determined using the TRIOS software version v3.1.5.3696. FIG. 5A shows the DSC first heating scan of the PET samples described in Examples 1-5 and Comparative Example 1, and FIG. 5B shows the DSC first heating scan of the rPET samples described in Examples 7-8 and Comparative Example 3. Table 3 summarizes the main results extracted from each DSC scan.

TABLE 3
Characterization of PET samples from DSC first heating scan

| | | T_{crystallization} | T_{melting} | | | |
|---|---|---|---|---|---|---|
| | | (mean, onset) (° C.) | (mean, onset) (° C.) | ΔH_{crystallization} (J/g) | ΔH_{melting} (J/g) | CD (%) |
| Example 1 | 76 | 124, 112 | 244, 225 | 23 | 40 | 12 |
| Example 2 | 74 | 129, 123 | 250, 232 | 23 | 41 | 13 |
| Example 3 | 75 | 126, 117 | 250, 231 | 25 | 41 | 11 |
| Example 4 | 76 | 126, 115 | 246, 226 | 23 | 39 | 11 |
| Example 5 | 75 | 137, 124 | 232, 206 | 20 | 27 | 5 |
| Comparative | 76 | 124, 118 | 252, 235 | 25 | 42 | 12 |
| Example 1 | | | | | | |
| Example 7 | 76 | 127, 109 | 242, 223 | 29 | 39 | 7 |
| Example 8 | 69 | 133, 113 | 230, 199 | 25 | 32 | 5 |
| Comparative | 78 | 129, 123 | 253, 236 | 27 | 45 | 13 |
| Example 3 | | | | | | |

### Example 10

Reactive Mixing/Extrusion of rPET and 1 wt. % DGT

A pretreated rPET was prepared and synthesized under the same conditions as described in Example 7 with the only exception that the composition of reactive agent DGT was 1 wt. %.

### Example 11

Pretreated rPET Synthesized by Reactive Extrusion/Mixing with DGT 1 wt. % Followed by Isothermal Annealing for 3 Minutes

A pretreated rPET was prepared and synthesized under the same conditions as described in Example 10 with the only exception that after reactive extrusion or reactive mixing and fast cooling, the obtained extrudate was placed in a 300 μm thick stainless steel mold and annealed at 280° C. for 3 minutes under vacuum using a hot plate vacuum desiccator. After the thermal annealing, the resulting material was fast cooled in a water bath at room temperature.

### Example 12

Pretreated rPET Synthesized by Reactive Extrusion/Mixing with DGT 1 wt. % Followed by Isothermal Annealing for 10 Min

A pretreated rPET was prepared and synthesized under the same conditions as described in Example 11 with the only exception the obtained extrudate was annealed at 280° C. for 10 minutes. After the thermal annealing, the resulting material was fast cooled in a water bath at room temperature.

The following examples illustrate the preparation of pretreated PET by thermally annealing for longer times.

### Example 13

Pretreated PET Synthesized by Reactive Extrusion/Mixing with 5 wt. % DGT Followed by Isothermal Annealing for 1 Hour at 280° C.

A pretreated PET was prepared and synthesized under the same conditions as described in the Example 1. Samples of 3 mm thickness were prepared by pressing the pretreated PET at 150° C. for 30 sec. at 100 bar using a steel mold. The pretreated PET was thermally annealed for 1 hour at 280° C. using a TA ARES G2 analyzer operated with a 25 mm diameter parallel plate geometry under an air flow of 7 L/min. The variations of storage modulus (G′) and loss modulus (G″) as a function of time were recorded at an angular frequency of 1 rad·s⁻¹ and 0.5% strain. After the thermal annealing, the resulting material was fast cooled in a water bath at room temperature.

### Example 14

Pretreated rPET Synthesized by Reactive Extrusion/Mixing with 5 wt. % DGT Followed by Isothermal Annealing for 1 Hour at 280° C.

A pretreated rPET was prepared and synthesized under the same conditions as described in the Example 7. Samples of 3 mm thickness were prepared by pressing the pretreated rPET at 150° C. for 30 sec. at 100 bar using a steel mold. The pretreated rPET was thermally annealed for 1 hour at 280° C. using a TA ARES G2 analyzer operated with a 25 mm diameter parallel plate geometry under an air flow of 7 L/min. The variations of storage modulus (G′) and loss modulus (G″) as a function of time were recorded at an angular frequency of 1 rad·s⁻¹ and 0.5% strain. After the thermal annealing, the resulting material was fast cooled in a water bath at room temperature.

### Example 15

Pretreated rPET Synthesized by Reactive Extrusion/Mixing with 1 wt. % DGT Followed by Isothermal Annealing for 1 Hour at 280° C.

A pretreated rPET was prepared and synthesized under the same conditions as described in the Example 10. Samples of 3 mm thickness were prepared by pressing the pretreated rPET at 150° C. for 30 sec. at 100 bar using a steel mold. The pretreated rPET was thermally annealed for 1 hour at 280° C. using a TA ARES G2 analyzer operated with a 25 mm diameter parallel plate geometry under an air flow of 7 L/min. The variations of storage modulus (G′) and loss modulus (G″) as a function of time were recorded at an angular frequency of 1 rad·s⁻¹ and 0.5% strain. After the thermal annealing, the resulting material was fast cooled in a water bath at room temperature.

### Rheometry

The following example illustrates the variations of linear shear complex modulus (G*) defined by Eq. 3 as a function of time for the pretreated PET of Examples 1, 5, 7, 8, 10, 11, 12, 13, 14, and 15 and Comparative Examples 1 and 3.

$${G}^{*} = \sqrt{{( {G}^{′} )}^{2} + {( {G}^{″} )}^{2}} \qquad ( 3 )$$

where G′ is the storage modulus and G″ is the loss modulus.

### Example 16

Samples of 3 mm thickness were prepared by pressing the pretreated PET at 150° C. for 30 sec. at 100 bar using a steel mold. The 3 mm thickness samples were characterized by rheometry using a TA ARES G2 analyzer operated with a 25 mm diameter parallel plate geometry at 280° C. under an air flow of 7 L/min. The variations of storage modulus (G′) and loss modulus (G″) as a function of time were recorded at an angular frequency of 1 rad·s⁻¹ and 0.5% strain for pretreated PET of the Example 1, Example 7 and Example 10 and at an angular frequency of 1 rad·s⁻¹ and 10% strain for pretreated PET of the Comparative Example 1 and Comparative Example 3. The variations of G′ and G″ as a function of time were also recorded at an angular frequency of 0.1 rad·s⁻¹, 0.5% strain and T=280° C. for pretreated PET of Example 1, Example 5, Example 7, and Example 8.

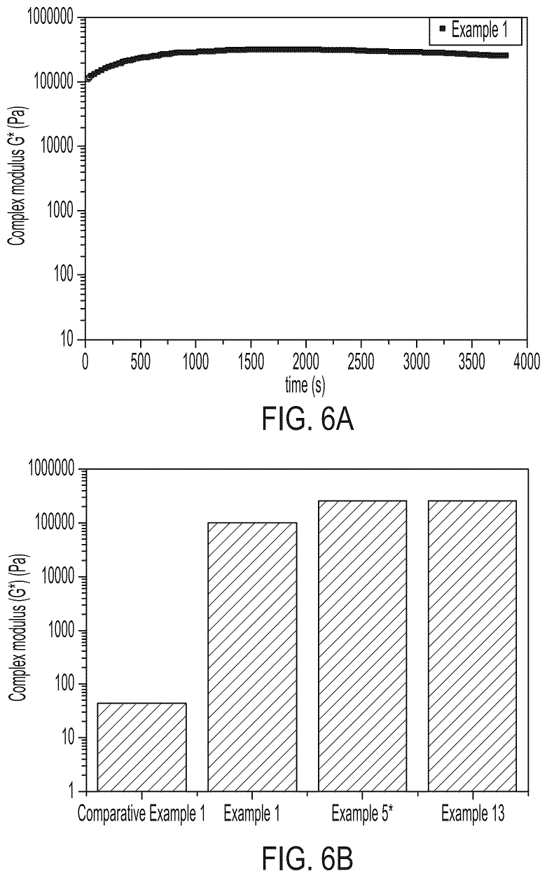

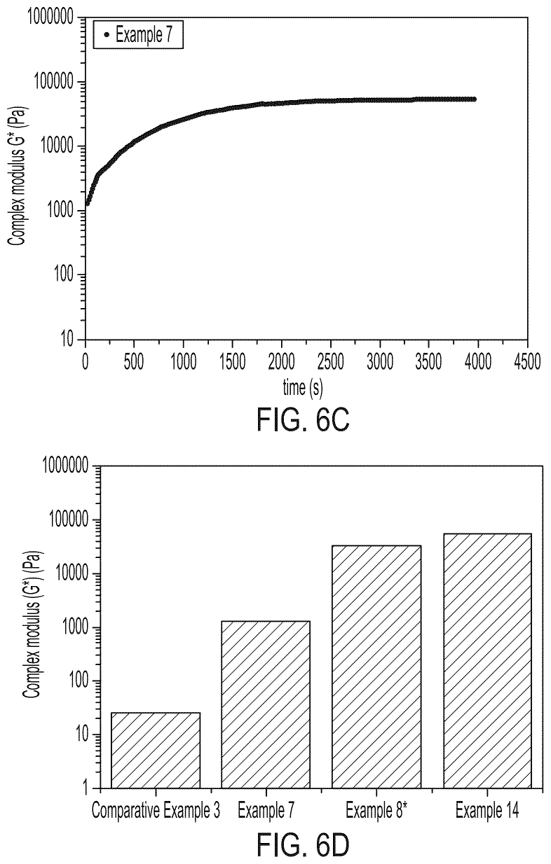

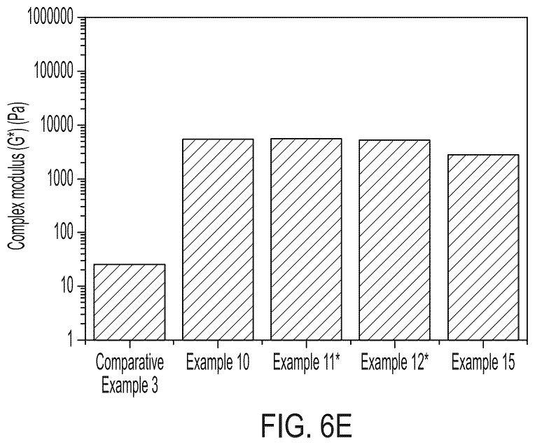

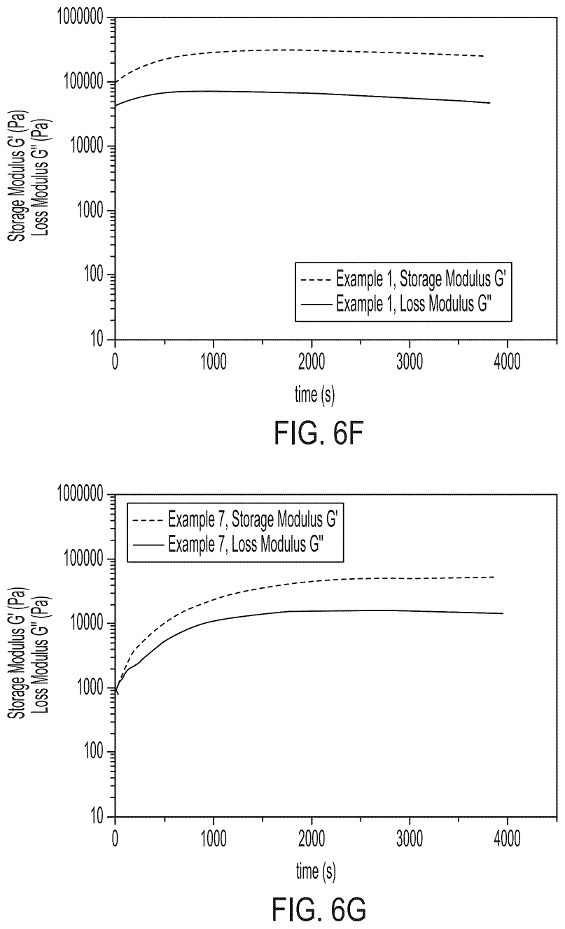

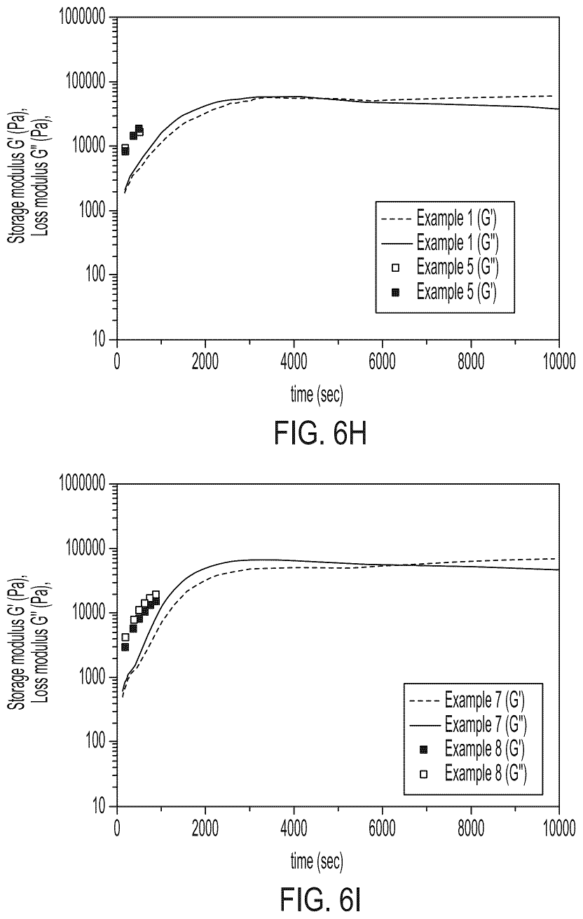

FIG. 6A shows the evolution of G* of pretreated PET prepared as described in Example 1.

FIG. 6B shows the values of G* corresponding to the pretreated PET of Comparative Example 1, Example 1, Example 13, and the equivalent of Example 5 extracted from the curve G* v. time corresponding to Example 1 after annealing for 10 minutes at 280° C.

FIG. 6C shows the evolution of G* of pretreated rPET prepared as described in Example 7.

FIG. 6D shows the values of G* corresponding to the pretreated rPET of Comparative Example 3, Example 7, Example 14, and the equivalent of Example 8 extracted from the curve G* v. time corresponding to Example 7 after annealing for 20 minutes at 280° C.

FIG. 6E shows the values of G* corresponding to the pretreated rPET of Comparative Example 3, Example 10, and Example 15. FIG. 6E also shows the values of G* corresponding to the pretreated rPET equivalents of Example 11 and Example 12 extracted from the curve G* v. time corresponding to Example 10 after annealing for 3 minutes at 280° C. and 10 minutes at 280° C., respectively.

FIG. 6F shows the evolution of shear storage modulus G′ and shear loss modulus G″ over time at an angular frequency of 1 rad·s⁻¹, 0.5% strain, and T=280° C. for the PET sample described in Example 1.

FIG. 6G shows the evolution of shear storage modulus G′ and shear loss modulus G″ over time at an angular frequency of 1 rad·s⁻¹, 0.5% strain and T=280° C. for the rPET sample described in Example 7.

FIG. 6H shows the evolution of shear storage modulus G′ and shear loss modulus G″ over time at an angular frequency of 0.1 rad·s⁻¹, 0.5% strain and T=280° C. for the PET samples described in Example 1 and Example 5.

FIG. 6I shows the evolution of shear storage modulus G′ and shear loss modulus G″ over time at an angular frequency of 0.1 rad·s⁻¹, 0.5% strain and T=280° C. for the rPET samples described in Example 7 and Example 8.

The advancement of reactions of chain extension/branching/cross-linking is evidenced by an increase of G* for pretreated PET or pretreated rPET compared to non-pretreated PET or non-pretreated rPET, respectively. Thermal annealing of pretreated PET of Example 1 and Example 7 at the same temperature and time annealing conditions used to obtain the pretreated PET of Example 5 and Example 8, respectively, produces an additional increase of G* which evidences a higher cross-linking degree.

The following example illustrates the characterization of properties of pretreated PET and rPET materials by DSC by following a well-defined sample preparation protocol, which enables a reproducible characterization for any post-consumer and/or post-industrial plastic material.

Sample Preparation Protocol: Around 5 mg of the pretreated post-consumer polymeric material were weighed in a DSC capsule (TA, Tzero Pan T 220228 and Tzero Hermetic Lid T 220315). The capsule with the pretreated post-consumer polymeric material was heated in an oven at a temperature of Tₘ+30° C. (Tₘ=melting temperature of the crystallizable polymer) for 3 minutes. The melted pretreated post-consumer polymeric material was then fast cooled at least at T_{g}−50° C. or lower by immersing the capsule into an iced water bath (5° C.) for 1-2 seconds. The capsule was wiped with a paper tissue and dried with an air flow at room temperature. The final mass (after drying) of the capsule containing the pretreated post-consumer plastic was measured to confirm it matched the initial mass (before heating 3 minutes in an oven) of the capsule containing the pretreated post-consumer polymeric material.
DSC scan protocol: Characterization by DSC of pretreated post-consumer and/or post-industrial polymeric material was performed by the following sequence of steps to samples obtained by the Sample preparation protocol:
1) Equilibrate temperature at least at T=(T_{g} of the crystallizable polymer or copolymer−30° C.);
2) Isotherm at temperature of step 1 for 1 minute;
3) Heat from temperature of step 2 to T=(Tₘ of crystallizable polymer or copolymer+40° C.) at a heating rate of 10° C./min;
4) Isotherm at temperature of step 3 for 3 minutes;
5) Cool from temperature of step 4 to at least T=(T_{g} of crystallizable polymer or copolymer−30° C.) at a cooling rate of 20° C./min;
6) Isotherm at temperature of step 5 for 1 min;
7) Heat from temperature of step 6 to T=(Tₘ+40° C.) at a heating rate of 10° C./min;

### Example 17

In this example, the pretreated PET and rPET synthesized in Example 1, Example 7, Example 8, Example 10, Example 12, Example 13, Example 14, Example 15 and Comparative Example 1 and Comparative Example 3 were characterized by DSC. Around 5 mg of the pretreated PET and rPET were weighed in a DSC capsule (TA, Tzero Pan T 220228 and Tzero Hermetic Lid T 220315). The capsule with the pretreated PET or pretreated rPET was heated in an oven at 280° C. (Tₘ+30° C.) for 3 minutes. The melted pretreated PET or pretreated rPET was then fast cooled by immersing the capsule into an iced water bath (5° C.) for 1-2 seconds. The capsule was wiped with a paper tissue and dried with an air flow at room temperature. The final mass (after drying) of the capsule containing the pretreated PET or pretreated rPET was measured to confirm it matched with the initial mass (before heating 3 min in an oven) of the capsule containing the pretreated PET or pretreated rPET.

The DSC scans were measured using the following sequence of steps: 1) Equilibrate temperature at 0° C.; 2) Isotherm at 0° C. for 1 min; 3) Heat from 0° C. to 290° C. at a heating rate of 10° C./min; 4) Isotherm at 290° C. for 3 min; 5) Cool from 290° C. to 0° C. at a cooling rate of 20° C./min; 6) Isotherm at 0° C. for 1 min; 7) Heat from 0° C. to 290° C. at a heating rate of 10° C./min.

Normalized heat flow v. temperature curves were analyzed using TRIOS software version v3.1.5.3696. Glass transition temperature in the first (T_{g}1) and the second (T_{g}2) heating scan were determined as the midpoint of the transition. Crystallization temperature in the first (Tc1) and the second (Tc2) heating scan were determined as the peak temperature of the exothermic peak at around (110-160° C.) Crystallization enthalpy in the first (ΔHc1) and the second (ΔHc2) heating scan were obtained by integration of the exothermic peak at around (110-160° C.) The integration was performed from visually determined respective starting points to end points using a straight baseline. Melting point in the first (Tm1) and the second (Tm2) heating scan were taken as the peak temperature of the endothermic peak in the range (200-250° C.) Melting enthalpy in the first (ΔHm1) and the second (ΔHm2) heating scan were obtained by integration of the endothermic peak at around (200-250° C.) The integration was performed from visually determined respective starting points to end points using a straight baseline. Crystallization temperature from the melt (Tcfm) in the cooling scan was taken as the peak temperature of the exothermic peak at around (110-210° C.) Crystallization enthalpy from the melt (ΔHcfm) in the cooling scan was obtained by integration of the exothermic peak at around (110-210° C.) The integration was performed from visually determined respective starting points to end points using a straight baseline.

Table 4 summarizes the main results extracted from each DSC scan.

TABLE 4
Main results extracted from each DSC scan described in the Example 17.

| | First heating scan | | | | | Cooling scan | | Second heating scan | | | | |
|---|---|---|---|---|---|---|---|---|---|---|---|---|
| | (10° C./min) | | | | | (20° C./min) | | (10° C./min) | | | | |
| Example | Tg1 | Tc1 | ΔHc1 | Tm1 | ΔHm1 | Tcfm | ΔHcfm | Tg2 | Tc2 | ΔHc2 | Tm2 | ΔHm2 |
| Example 1 | 76 | 132 | 34 | 242 | 39 | 142 | 5 | 76 | 141 | 23 | 234 | 33 |
| Comparative | 70 | 118 | 34 | 250 | 47 | 204 | 50 | 75 | — | — | 250 | 45 |
| Example 1 | | | | | | | | | | | | |
| Example 13 | 76 | 138 | 27 | 230 | 34 | 145 | 9 | 75 | 138 | 19 | 231 | 34 |
| Comparative | 75 | 120 | 34 | 249 | 49 | 204 | 51 | 76 | — | — | 250 | 47 |
| Example 3 | | | | | | | | | | | | |
| Example 7 | 67 | 115 | 33 | 239 | 40 | 168 | 38 | 77 | — | — | 233 | 37 |
| Example 8 | 68 | 132 | 26 | 230 | 32 | 144 | 11 | 76 | 142 | 15 | 232 | 31 |
| Example 14 | 77 | 140 | 23 | 224 | 26 | 139 | 2 | 77 | 143 | 21 | 225 | 26 |
| Example 10 | 77 | 126 | 31 | 246 | 38 | 197 | 47 | 76 | — | — | 247 | 44 |
| Example 12 | 74 | 123 | 33 | 247 | 47 | 199 | 48 | 77 | — | — | 247 | 45 |
| Example 15 | 65 | 120 | 40 | 244 | 51 | 198 | 51 | 74 | — | — | 245 | 48 |

### Isothermic DSC

The following examples illustrate that the pretreatment by reactive extrusion and/or reactive mixing with the reactive agent followed by an annealing step retards the crystallization process of PET.

### Comparative Example 4

A PET sample was prepared following the same procedure as described in Comparative Example 1. The extrudate was cut into pieces of 1 cm length. Cut PET pieces (10 g) were placed in a beaker, and the beaker was immersed into liquid nitrogen for 1 minute. Then the cooled PET pellets were transferred to a Moulinex grinder (50 g, 180 W) and milled for 1 minute. The obtained powder was transferred to the beaker and cooled for 1 minute by immersing it in liquid nitrogen. Then the cooled powder was transferred to the Moulinex grinder and milled for 1 min. The milled sample was fractionated by sieving using an analytical sieve shaker (Retsch, AS200) operating at an amplitude of about 3 mm for 2 cycles of 10 minutes (20 minutes of total shaking). Fractionation was performed using stainless steel test sieves (Retsch) with a diameter of 100 mm and mesh sizes of: 300 μm, 150 μm, 100 μm, and 36 μm. The micronized PET fraction obtained between the 150 μm and 300 μm mesh sizes was used for the isothermic DSC as follows. 200 mg of micronized PET (150-300 μm) was weighed in a glass vial and 20 mL of potassium phosphate buffer 1 M was added. The particles were soaked in the aqueous solution at 70° C. for 1 hour. Soaked particles were separated by filtration and wiped with a paper tissue to remove the excess water. PET soaked particles (15 mg) were incorporated into a DSC capsule (TA, Tzero Pan T 220228 and Tzero Hermetic Lid T 220315) and 20 μL of potassium phosphate buffer 1 M (pH 8) were added into the capsule. The capsule was hermetically closed and placed into the DSC to run the following sequence: 1) Equilibrate temperature at 30° C.; 2) Isothermal step (30° C.) for 1 min; 3) Heating step from 30° C. to 75° C. at a heating rate of 10° C./min. The heat flow was monitored as a function of incubation time at 75° C.

### Example 18

Isothermic DSC was performed under the same conditions described in Comparative Example 4 with the only exception that the PET sample was synthesized as described in Example 5 (pretreated PET by reactive extrusion/mixing with 5 wt. % DGT followed by isothermal annealing).

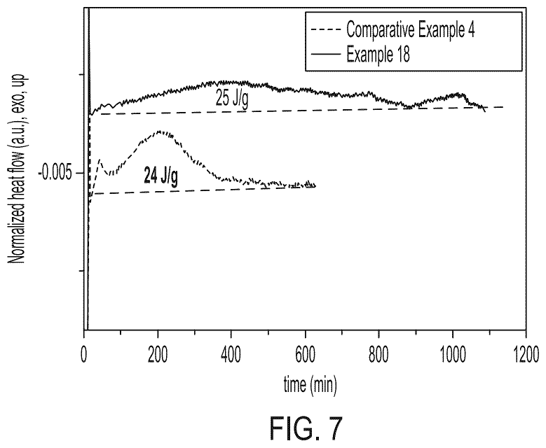

FIG. 7 shows the normalized heat flow as a function of incubation time at 75° C. for samples prepared as described in Comparative Example 4 and Example 18. The exothermic peak for both samples corresponds to the crystallization of PET. Surprisingly, the pretreatment of PET by reactive extrusion or reactive mixing/thermal annealing slowed down the crystallization process. In Comparative Example 4, crystallization finished in about 7 hours at 75° C., whereas in Example 18, crystallization finished in about 15 hours under the same experimental conditions.

Isothermal DSC protocol: The isothermal DSC characterization of pretreated post-consumer and/or post-industrial polymeric material was performed by the following sequence of steps to samples obtained by the Sample Preparation Protocol:
1) Equilibrate temperature at least T_{g}−30° C. (T_{g} of the crystallizable polymer or copolymer);
2) Isotherm at temperature of step 1 for 1 minute;
3) Heat from temperature of step 2 to T_{g}+30° C. (T_{g} of the crystallizable polymer or copolymer) at a heating rate of 10° C./min;
4) Isotherm at T_{g}+30° C. for at least 120 minutes.

The time of crystallization of the pretreated post-consumer and/or post-industrial polymeric material at T=T_{g}+30° C. was obtained as the peak time of the exothermic peak in the heat flow v. time curve corresponding to step 4 of the Isothermal DSC protocol. The t=0 is the starting time of step 1 of the Isothermal DSC protocol.

### Example 19

In this Example, the crystallization time of the pretreated PET and rPET synthesized in Example 1, Example 7, Example 8, Example 10, Example 12, Example 13, Example 14, Comparative Example 1, and Comparative Example 3 were characterized by DSC at a measurement temperature of T_{g}+30° C.=105° C. using a calorimeter (TA, discovery Q200).

Around 5 mg of the pretreated PET and rPET were weighed in a DSC capsule (TA, Tzero Pan T 220228 and Tzero Hermetic Lid T 220315). The capsule with the pretreated PET or pretreated rPET was heated in an oven at 280° C. (Tₘ+30° C.) for 3 minutes. The melted pretreated PET or pretreated rPET was then fast cooled by immersing the capsule into an iced water bath (5° C.) for 1-2 seconds. The capsule was wiped with a paper tissue and dried with an air flow at room temperature. The final mass (after drying) of the capsule containing the pretreated PET or pretreated rPET was measured to confirm it matched the initial mass (before heating 3 minutes in an oven) of the capsule containing the pretreated PET or pretreated rPET. Isothermal DSC were measured using the following sequence of steps: 1) Equilibrate temperature at 30° C.; 2) Isotherm at 30° C. for 1 minute; 3) Heat from 30° C. to 105° C. at a heating rate of 10° C./min; 4) Isotherm at 105° C. for 120 minutes.

The time of crystallization of the pretreated PET or pretreated rPET at 105° C. was obtained as the peak time of the exothermic peak in the heat flow v. time curve, considering t=0 the starting time of the step 1 of the isothermal DSC protocol.

Table 5 summarizes the time of crystallization of the pretreated PET and rPET for Example 1, Example 7, Example 8, Example 10, Example 12, Example 13, Example 14, Comparative Example 1, and Comparative Example 3.

TABLE 5
Time of crystallization of pretreated PET and rPET at 105° C.

| | Time of crystallization at |
|---|---|
| Example | Tg + 30° C. = 105° C. (min) |
| Example 7 | 15 |
| Example 8 | 25 |
| Example 14 | 31 |
| Comparative Example 3 | 13 |
| Example 1 | 20 |
| Example 13 | 22 |
| Comparative Example 1 | 15 |
| Example 10 | 19 |
| Example 12 | 15 |

### Example 20

### FT-IR

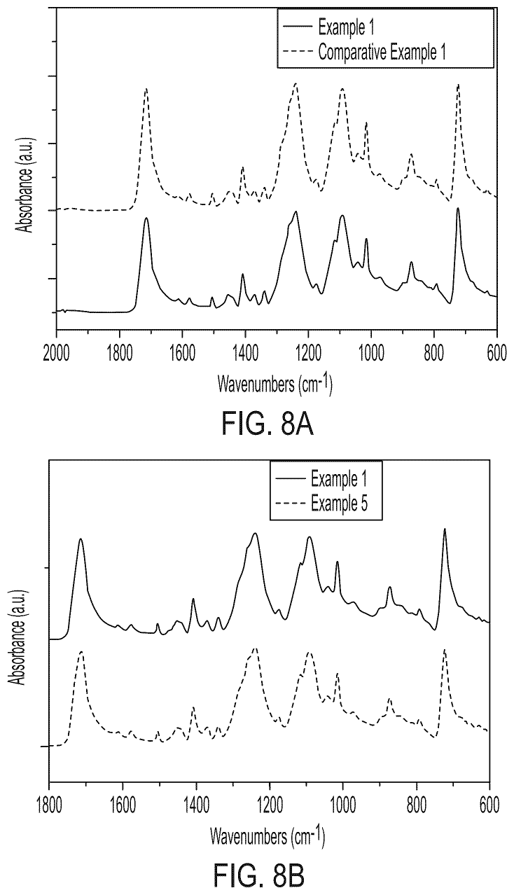

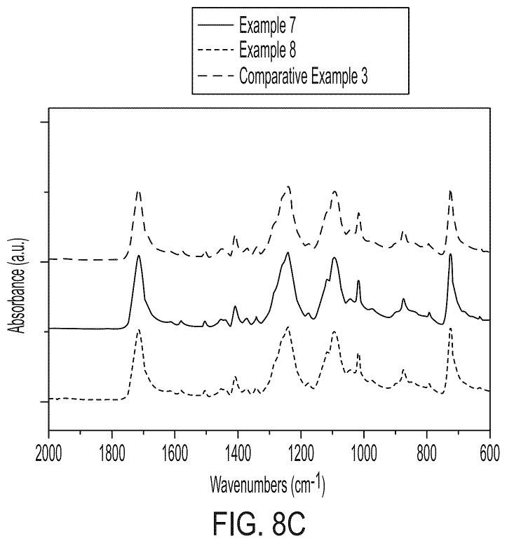

Fourier Transform Infrared Spectroscopy-Attenuated Total Reflectance (FTIR-ATR) spectra were recorded using a Tensor 27 (Bruker) apparatus. The spectra were recorded with a resolution of 4 cm⁻¹ and a 32-scan accumulation. FIG. 8A shows the FTIR-ATR spectra of samples obtained by the conditions described in Example 1 and Comparative Example 1 in the range 2000-600 cm⁻¹. There were no significant changes in the spectra after reactive extrusion or reactive mixing with DGT (5 wt. %) under the conditions described in Example 1. Given that the reactive structure matches that of the polymer backbone, the incorporation of DGT into the polymer chains by the reactions described in FIGS. 2A-2D did not lead to significant changes in the structure, as observed by FT-IR.

FIG. 8B shows the FTIR-ATR spectra of samples obtained by the conditions described in Examples 1 and 5 in the range 2000-600 cm⁻¹. The isothermal annealing process after reactive extrusion or reactive mixing did not produce any significant difference in the FT-IR spectra, as expected under the basis of the cross-linking reactions described in FIG. 3.

FIG. 8C shows the FTIR-ATR spectra of samples obtained by the conditions described in Examples 7 and 8 and Comparative Example 3 in the range 2000-600 cm⁻¹. The isothermal annealing process after reactive extrusion or reactive mixing did not produce any significant difference in the FT-IR spectra, as expected under the basis of the cross-linking reactions described in FIG. 3.

### Example 21

### Terephthalic Acid (TPA) Production

During the enzymatic depolymerization of PET, the monomer TPA is released into the solution as a product. Given that the chemical structure of DGT matches the chemical structure of the polymer backbone, the enzymatic degradation of pretreated PET as described in Examples 1 and 5 and pretreated rPET as described in Examples 7 and 8 releases the TPA monomer.

Reaction progress was followed by measuring the absorbance of the aqueous solution as a function of digestion time by means of a Clariostar LVis plate (BMG Labtech). Aliquots of 2 μL were taken at regular time intervals. Before measuring the absorbance, the aliquots ware diluted in NaOH 0.5 wt. % solution, with a dilution factor in the range X10 to X100 depending of incubation time. The absorbance of diluted aliquots was measured on a Clariostar microplate. Spectra were recorded between 220 and 800 nm. The reaction yield was followed as the increase of the TPA absorbance at 242 nm (maximum absorbance of TPA), corrected by the corresponding dilution factor. Absorbance of diluted aliquots was measured at least by duplicate and the average absorbance was taken to follow the reaction yield. The average absorbance was converted into TPA equivalents using a calibration curve (Absorbance at 242 nm v. TPA concentration), obtained by measuring the absorbance of TPA aqueous solutions of known concentrations. Given that at 100% yield of enzymatic depolymerization of PET (x mg) the TPA concentration is x*[Molecular weight of TPA]/[Molecular weight of PET repeating unit]=x*166/192, for enzymatic depolymerization experiments starting from 25 mg of PET, the TPA equivalent concentration at 100% is 21.7 g/L, and for enzymatic depolymerization experiments starting from 5 mg of PET, the TPA equivalent concentration at 100% is 4.3 g/L.

The following example describes the quantification of catalyst(s) already present in the post-consumer PET flakes (PolyQuest) used to synthesize the pretreated materials of Example 7, Example 8, Example 10, Example 11, Example 12, Example 14, Example 15, and Comparative Example 3.

### Example 22

The metal catalyst(s) composition present in the post-consumer PET flakes (rPET) used in Example 7, Example 8, Example 10, Example 11, Example 12, Example 14, Example 15, and Comparative Example 3 was determined and quantified by the analytical technique inductively coupled plasma atomic emission spectroscopy (ICP-AES). Table 6 summarizes the results of the analysis.

TABLE 6
Results of the quantification of metal catalyst(s) present
the post-consumer PET flakes (rPET) used in Example
7, Example 8, Example 10, Example 11, Example 12, Example
14, Example 15, and Comparative Example 3

| Element | Technique | Result (mg/kg) |
|---|---|---|
| Titanium (Ti) | ICP-AES | 3.7 |
| Zinc (Zn) | ICP-AES | <1.0 |
| Germanium (Ge) | ICP-AES | <1.0 |
| Antimony (Sb) | ICP-AES | 312 |

### Digestion Data

The following examples illustrate that the pretreatment of PET by reactive extrusion or reactive mixing/thermal annealing in the presence of a suitable reactive agent enables a more efficient enzymatic depolymerization of the crystallizable plastic, as measured by a faster depolymerization rate and a surprisingly higher depolymerization yield.

Enzymatic Activity with HiC Novozym at T=75° C.
Post-Consumer PET (rPET)

### Example 23

Pretreated rPET by Reactive Extrusion/Mixing with 5 wt. % DGT

A pretreated rPET was prepared following the same conditions described in Example 7. The extrudate was cut into pieces of 3-5 mm length. Cut rPET pieces (2 g) were micronized using a centrifugal mill (Retsch ZM200) operating at 14000 RPM in two milling steps. The first milling step was performed with a ring sieve having an internal diameter of 10 cm and a mesh size of 0.5 mm. The obtained micronized rPET was collected and subjected to a second milling step using a ring sieve with a mesh size of 0.25 mm and an internal diameter of 10 cm. Feed rate in both milling steps was about 1 g/min, and the maximum ring sieve temperature (measured by attaching a type K thermocouple to the external wall of the ring sieve) was below 40° C.

The milled sample was fractionated by sieving using an analytical sieve shaker (Retsch, AS200) operating at an amplitude of about 3 mm for 2 cycles of 10 minutes (20 minutes of total shaking). Fractionation was performed using stainless steel test sieves (Retsch) with a diameter of 100 mm and mesh sizes of: 300 μm, 150 μm, 100 μm and 36 μm. The micronized PET fraction obtained between the 150 μm and 300 μm mesh sizes was used for the enzymatic activity essays as follows.

25 mg of PET milled and sieved fraction were added in a 2 mL Eppendorf with 1 mL of potassium phosphate buffer 1 M. The Eppendorf was cooled in ice. The enzymatic activity tests were performed with a commercially available thermostable cutinase (HiC, Novozym 51032). HiC Novozym 51032 solution (6 g/L) was added in the Eppendorf to give a final concentration of 2 mg enzyme/g of PET. The Eppendorf was incubated in a Thermomixer (Eppendorf) at 75° C. with shaking at 1200 RPM. Reaction progress was followed by measuring the absorbance by means of a Clariostar LVis plate (BMG Labtech). Aliquots of 2 μL were taken at regular time intervals. Before measuring the absorbance, the aliquots taken during the first 1.5 h of incubation time were diluted by 10 in NaOH 0.5 wt. % solution. The aliquots taken at incubation times longer than 1.5 h were diluted by a factor of 100 in NaOH 0.5 wt. % solution. The absorbance of diluted aliquots was measured on a Clariostar microplate. Spectra were recorded between 220 and 800 nm and the enzymatic depolymerization essays were performed by triplicate. The average absorbance at 242 nm (corrected by the dilution factor) was taken to extract the TPA equivalent (g/L) as a function of the depolymerization reaction time using the calibration curve (Absorbance v. TPA concentration): TPA equivalent=(Absorbance mean*25)/(70.47*real mass of PET). The reaction yield as a function of reaction time was obtained from the TPA equivalent as: Reaction yield (%)=(100*TPA equivalent)/21.7. Error bars of reaction yield v. reaction time correspond to the standard deviation.

### Example 24

Pretreated rPET by Reactive Extrusion/Mixing with 5 wt. % DGT Followed by Isothermal Annealing

A pretreated rPET was prepared following the same conditions described in Example 8. Micronization, fractionation of sample, and an enzymatic depolymerization activity test were performed under the same conditions as described in Example 23. Measurements of enzymatic depolymerization were performed in triplicate.

### Comparative Example 5

rPET by Extrusion and Fast Cooling

An rPET sample was prepared following the same procedure as described in Comparative Example 3. Micronization, fractionation, and an enzymatic depolymerization activity test were performed following the same conditions as described in Example 23. Measurements of enzymatic depolymerization were performed in triplicate.

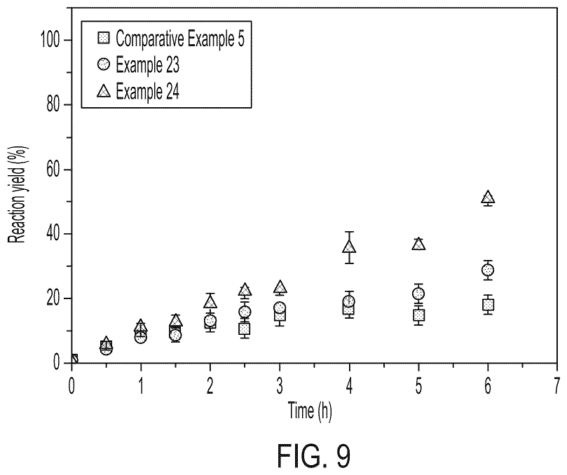

From FIG. 9, which shows the reaction yield v. incubation time for the pretreated rPET of Examples 23 and 24 and the non-pretreated rPET of Comparative Example 5, it is evidenced that both the rate of enzymatic depolymerization and the reaction yield increase when rPET is pretreated by reactive extrusion or reactive mixing with reactive agent DGT. Notably, the effect is more significant when the pretreatment by reactive extrusion or reactive mixing with reactive agent DGT is followed by isothermal annealing.

In the following examples, enzymatic activity tests were performed using a more efficient enzyme to depolymerize PET compared to HiC Novozym 51032.

Enzymatic activity with an LCC variant at T=65° C.

### Example 25

Pretreated PET by Reactive Extrusion/Mixing with 5 wt. % DGT

A pretreated PET was prepared following the same conditions described in Example 1. The extrudate was cut into pieces of 1 cm length. Cut PET pieces (10 g) were placed in a beaker and the beaker was immersed into liquid nitrogen for 1 minute. Then the cooled PET pellets were transferred to a Moulinex grinder (50 g, 180 W) and milled for 1 minute. The obtained powder was transferred to the beaker and cooled for 1 minute by immersing it in liquid nitrogen. Then the cooled powder was transferred to the Moulinex grinder and milled for 1 minute. The milled sample was fractionated by sieving using an analytical sieve shaker (Retsch, AS200) operating at an amplitude of about 3 mm for 2 cycles of 10 minutes (20 minutes of total shaking). Fractionation was performed using stainless steel test sieves (Retsch) with a diameter of 100 mm and mesh sizes of: 300 μm, 150 μm, 100 μm and 36 μm. The micronized PET fraction obtained between the 150 μm and 300 μm mesh sizes was used for the enzymatic activity essays as follows.

25 mg of PET milled and sieved fraction were added in a 2 mL Eppendorf with 1 mL of potassium phosphate buffer 1 M. The Eppendorf was cooled in ice. The enzymatic activity tests were performed with a variant of the leaf-branch compost cutinase (LCC) having desirable thermostability and polyester degrading activity, referred to herein as “LCC variant”.

27.8 μL of stock solution (60 μM) of enzyme the LCC variant, having a molecular weight of 30 kDa, were added in the Eppendorf. Thus the concentration of enzyme in the Eppendorf was 1.67 μM (2 mg/g of PET). The Eppendorf was incubated in a Thermomixer (Eppendorf) at 65° C. with shaking at 1200 RPM.

Reaction progress was followed by measuring the absorbance by means of a Clariostar LVis plate (BMG Labtech).

Aliquots of 2 μL were taken at regular time intervals. Before measuring the absorbance, the aliquots taken during the first 1.5 hours of incubation time were diluted by 10 in NaOH 0.5 wt. % solution. The aliquots taken at incubation times longer than 1.5 hours were diluted by a factor of 100 in NaOH 0.5 wt. % solution. The absorbance of diluted aliquots was measured on a Clariostar microplate. Spectra were recorded between 220 and 800 nm and the enzymatic depolymerization assays were performed in duplicate. The average absorbance at 242 nm (corrected by the dilution factor) was taken to extract the TPA equivalent (g/L) as a function of the depolymerization reaction time using the calibration curve (Absorbance v. TPA concentration): TPA equivalent=(Absorbance mean*25)/(70.47*real mass of PET). The reaction yield as a function of reaction time was obtained from the TPA equivalent as: Reaction yield (%)=(100*TPA equivalent)/21.7. Error bars of reaction yield v. reaction time correspond to the difference of reaction yield by duplicate experiments.

### Example 26

Pretreated PET by Reactive Extrusion/Mixing with 5 wt. % DGT Followed by Isothermal Annealing

A pretreated PET was prepared following the same conditions described in Example 5. Micronization, fractionation of sample, and an enzymatic depolymerization activity test were performed under the same conditions as described in Example 25.

### Comparative Example 6

### PET by Extrusion and Fast Cooling

A PET sample was prepared following the same procedure as described in Comparative Example 1. Micronization, fractionation, and an enzymatic depolymerization activity test were performed following the same conditions as described in Example 25.

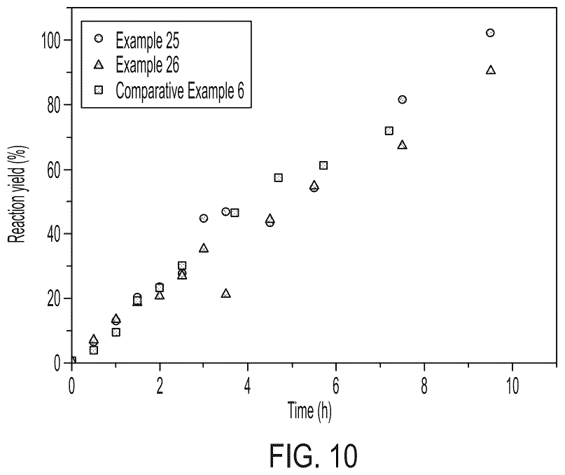

FIG. 10 shows the enzymatic depolymerization activity of milled particles of Example 25, Example 26, and Comparative Example 6. The reaction yield v. incubation time does not show significant differences between the pretreated material of Example 25 and Example 26 and the pretreated material of Comparative Example 6, which indicates that the pretreatment by reactive mixing/reactive extrusion with the reactive agent DGT does not reduce the rate and yield of enzymatic depolymerization.

Enzymatic Activity with the LCC Variant T=75° C.
Post-Consumer PET (rPET)

The following examples illustrate that a significant increase in the enzymatic depolymerization rate and reaction yield can be achieved at a higher incubation temperature when the post-consumer polymeric substrate is pretreated by reactive extrusion or reactive mixing/thermal annealing with a suitable reactive agent.

### Example 27

Pretreated rPET by Reactive Extrusion/Mixing with 5 wt. % DGT

A pretreated rPET was prepared following the same conditions described in Example 23. Micronization and fractionation of sample were performed under the same conditions as described in Example 23.

5 mg of PET milled and sieved fraction were added in a 2 mL Eppendorf with 1 mL of potassium phosphate buffer 1 M. The Eppendorf was cooled in ice. The enzymatic activity tests were performed with the genetically modified enzyme the LCC variant.

The concentration of enzyme in the Eppendorf was 2 mg/g of PET. The Eppendorf was incubated in a Thermomixer (Eppendorf) at 75° C. with shaking at 1200 RPM.

Reaction progress was followed by measuring the absorbance by means of a Clariostar LVis plate (BMG Labtech).

Aliquots of 2 μL were taken at regular time intervals. Before measuring the absorbance, the aliquots taken during the first 5 hours of incubation time were diluted by 10 in NaOH 0.5 wt. % solution. The aliquots taken at incubation times longer than 5 hours were diluted by a factor of 20 in NaOH 0.5 wt. % solution. The absorbance of diluted aliquots was measured on a Clariostar microplate. Spectra were recorded between 220 and 800 nm and the enzymatic depolymerization assays were performed in triplicate. The average absorbance at 242 nm (corrected by the dilution factor) was taken to extract the TPA equivalent (g/L) as a function of the depolymerization reaction time using the calibration curve (Absorbance v. TPA concentration): TPA equivalent=(Absorbance mean*5)/(70.47*real mass of PET). The reaction yield as a function of reaction time was obtained from the TPA equivalent as: Reaction yield (%)=(100*TPA equivalent)/4.3. Error bars of reaction yield v. reaction time correspond to the standard deviation of triplicate experiments.

### Example 28

Pretreated rPET by Reactive Extrusion/Mixing with 5 wt. % DGT Followed by Isothermal Annealing

A pretreated rPET was prepared following the same conditions described in Example 24. Micronization and fractionation of sample were performed under the same conditions as described in Example 24. An enzymatic depolymerization activity test was performed under the same conditions as described in Example 27 with the only exception that the aliquots taken during the first 2 hours of incubation time were diluted by 10 in NaOH 0.5 wt. % solution and the aliquots taken at incubation times longer than 2 hours were diluted by a factor of 20 in NaOH 0.5 wt. % solution.

### Example 29

Pretreated rPET by Reactive Extrusion/Mixing with 5 wt. % DGT Followed by Isothermal Annealing for Longer Times

A pretreated rPET was prepared following the same conditions described in Example 14. Micronization and fractionation of sample were performed under the same conditions as described in Example 24. An enzymatic depolymerization activity test was performed under the same conditions as described in Example 28.

### Example 30

Pretreated rPET by Reactive Extrusion/Mixing with 1 wt. % DGT

A pretreated rPET was prepared following the same conditions described in Example 10. Micronization and fractionation of sample were performed under the same conditions as described in Example 24. An enzymatic depolymerization activity test was performed under the same conditions as described in Example 27.

### Example 31

Pretreated rPET by Reactive Extrusion/Mixing with DGT 1 wt. % Followed by Isothermal Annealing

A pretreated rPET was prepared following the same conditions described in Example 11. Micronization and fractionation of sample were performed under the same conditions as described in Example 24. An enzymatic depolymerization activity test was performed under the same conditions as described in Example 28.

### Comparative Example 7

rPET by Extrusion and Fast Cooling

A pretreated rPET was prepared following the same conditions described in Comparative Example 5. Micronization and fractionation of sample were performed under the same conditions as described in the Example 23. An enzymatic depolymerization activity test was performed under the same conditions as described in Example 27.

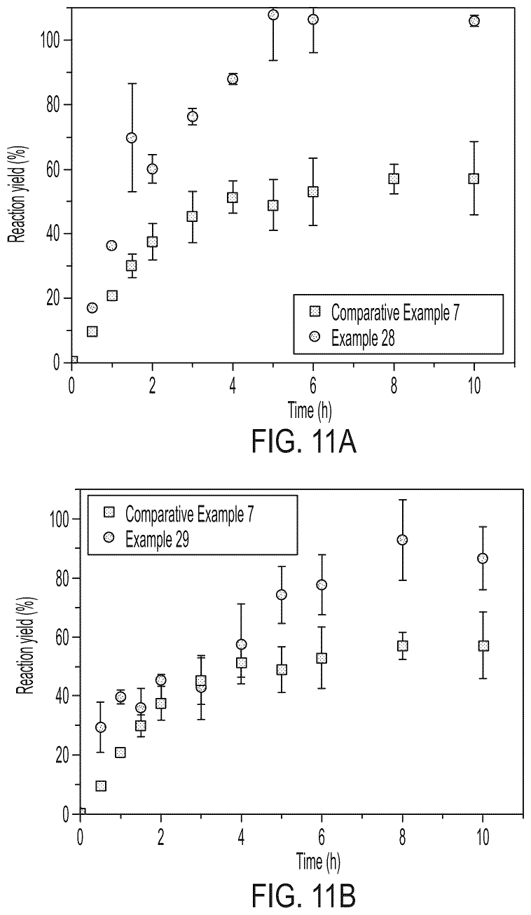

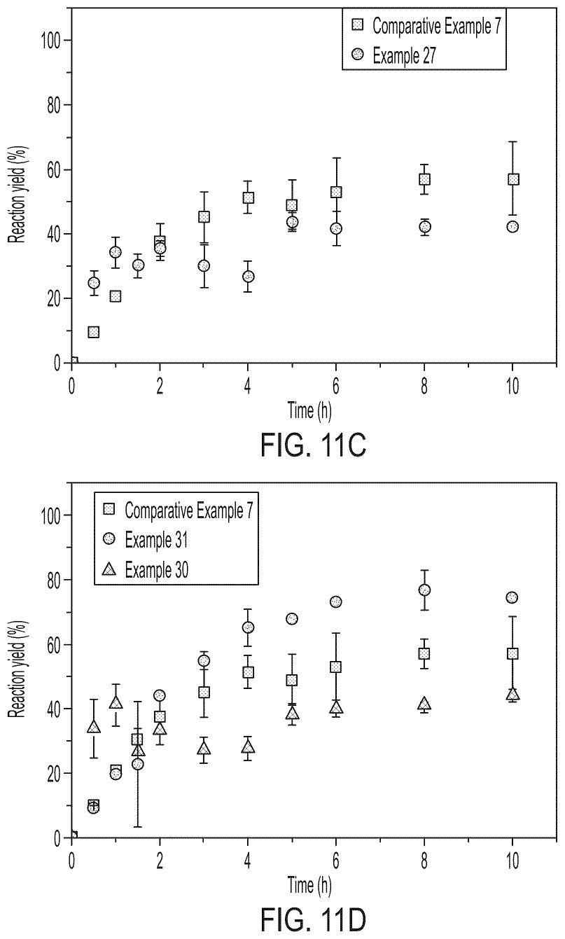

The final reaction yield obtained for the Comparative Example 7 (around 50%) (FIG. 11A) is similar to that previously reported in literature (Tournier et al., 2020) for the enzymatic depolymerization of rPET using an LCC variant and reaction temperature (75° C.). Surprisingly, the higher rate and much higher yield of reaction obtained here (around 100%) by the pretreatments described in the previous examples, in particular in Example 28 (FIG. 11A) and in Example 29 (FIG. 11B) represent a substantial improvement in the efficiency of enzymatic depolymerization of post-consumer PET using the same enzyme described before at 75° C.

FIG. 11C shows the enzymatic depolymerization v. incubation time for the pretreated rPET of Example 27 and Comparative Example 7 using the LCC variant at 75° C.

FIG. 11D shows the reaction yield v. incubation time for the pretreated rPET of Example 30, Example 31, and Comparative Example 7.

Enzymatic Activity with the LCC Variant T=85° C.

### Example 32

Pretreated rPET by Reactive Extrusion/Mixing with 5 wt. % DGT

A pretreated rPET was prepared following the same conditions described in Example 23. Micronization and fractionation of sample were performed under the same conditions as described in Example 23.

25 mg of PET milled and sieved fraction were added in a 2 mL Eppendorf with 1 mL of potassium phosphate buffer 1 M. The Eppendorf was cooled in ice. The enzymatic activity tests were performed with the genetically modified enzyme, the LCC variant, as described in Example 25.

27.8 μL of stock solution (60 μM) of the LCC variant, having a molecular weight of 30 kDa, were added in the Eppendorf. Thus the concentration of enzyme in the Eppendorf was 1.67 μM (2 mg/g of PET). The Eppendorf was incubated in a Thermomixer (Eppendorf) at 85° C. with shaking at 1200 RPM.

Reaction progress was followed by measuring the absorbance by means of a Clariostar LVis plate (BMG Labtech).

Aliquots of 2 μL were taken at regular time intervals. Before measuring the absorbance, the aliquots taken during the first 1.5 hours of incubation time were diluted by 10 in NaOH 0.5 wt. % solution. The aliquots taken at incubation times longer than 1.5 hours were diluted by a factor of 100 in NaOH 0.5 wt. % solution. The absorbance of diluted aliquots was measured on a Clariostar microplate. Spectra were recorded between 220 and 800 nm and the enzymatic depolymerization assays were performed in duplicate. The average absorbance at 242 nm (corrected by the dilution factor) was taken to extract the TPA equivalent (g/L) as a function of the depolymerization reaction time using the calibration curve (Absorbance v. TPA concentration): TPA equivalent=(Absorbance mean*25)/(70.47*real mass of PET). The reaction yield as a function of reaction time was obtained from the TPA equivalent as: Reaction yield (%)=(100*TPA equivalent)/21.7. Error bars of reaction yield v. reaction time correspond to the standard deviation of triplicate experiments.

### Example 33

Pretreated rPET by Reactive Extrusion/Mixing with 5 wt. % DGT Followed by Isothermal Annealing

A pretreated rPET was prepared following the same conditions described in Example 29. Micronization and fractionation of sample were performed under the same conditions as described in Example 29. Enzymatic depolymerization activity tests were performed under the same conditions as described in Example 32.

### Comparative Example 8

rPET by Extrusion and Fast Cooling

A pretreated rPET was prepared following the same conditions described in Comparative Example 7. Micronization and fractionation of sample were performed under the same conditions as described in Example 7. Enzymatic depolymerization activity tests were performed under the same conditions as described in Example 32.

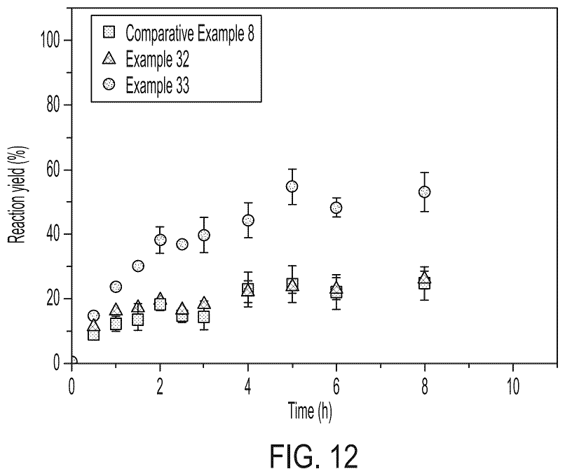

FIG. 12 shows the reaction yield v. incubation time for the pretreated rPET of Example 32, Example 33, and Comparative Example 8. It is observed that the pretreated rPET of Example 33 has a much higher reaction yield compared to the rPET of Comparative Example 8 and of Example 32, which indicates that the pretreatment of post-consumer rPET favors the efficiency of enzymatic depolymerization even at high temperatures.

### Example 34

Reactive Mixing/Extrusion of rPET and 5 wt. % DGT Followed by Isothermal Annealing at 280° C. for 20 Min without Pre-Drying rPET.

This example presents a synthesis of PC/IPM PET bottle waste for enzymatic degradation by reactive mixing/extrusion, fast cooling followed by isothermal annealing at 280° C. for 20 min without pre-drying rPET flakes. DGT diepoxy having a functionality f=4 is used as reactive agent.

PC/IPM PET flakes (rPET) (PolyQuest, https://www.polyquest.com/products/pet-and-rpet/) were used as received (not pre-dried). rPET flakes (11.4 g), reactive agent DGT (Denacol EX-711 Nagase ChemteX Corporation, 5 wt. %, 600 mg, 0.379 mmol epoxy/g rPET) and antioxidant (0.1 wt. %, Irganox 1010, Sigma) were fed into a hot conical twin screw compounder (DSM, Xplore, 15 cm³ capacity) equipped with co-rotating conical screws, recirculation channel allowing mixing during a controlled residence time and a circle die with diameter of 3.0 mm allowing for extrusion of the material from the compounder. The feeding/mixing/extrusion were performed under circulation of nitrogen, with a barrel temperature profile as follows: top position (270° C.), middle position (270° C.), and exit position (280° C.). The speed of rotation of the screws was 60 RPM. The extruder was filled in around 1.5 min. After feeding the extruder, the compound was mixed until the axial force reached 7000N. Then the sample was withdrawn directly through the die, extruded into an ice/water bath kept at 5° C.

The obtained extrudate was wiped with a paper tissue to remove water residue, was placed in a 1.5 mm thickness stainless steel mold and was annealed in an oven at 280° C. for 20 minutes. After thermal annealing, the resulting material was fast cooled in a water bath at room temperature.

The extrudate was cut into pieces of 3-5 mm length. Cut rPET pieces (2 g) were micronized using a centrifugal mill (Retsch ZM300) operating at 14000 RPM, cyclone-suction system for efficient air cooling and a ring sieve having an internal diameter of 10 cm and a mesh size of 0.5 mm. Feed rate was about 1 g/min, and the maximum ring sieve temperature (measured by attaching a type K thermocouple to the external wall of the ring sieve) was below 40° C. The milled sample was fractionated by sieving using an analytical sieve shaker (Retsch, AS200) operating at an amplitude of 0.7 mm for 10 minutes. Fractionation was performed using stainless steel test sieves (Retsch) with a diameter of 100 mm and mesh sizes of: 300 μm and 150 μm. The micronized PET fraction obtained between the 150 μm and 300 μm mesh sizes was used for enzymatic degradation tests.

### Comparative Example 9

rPET by Extrusion and Fast Cooling without a Pre-Drying Step of rPET Flakes.

The following example describes the preparation of rPET following standard extrusion followed by fast cooling in the absence of a reactive agent without a pre-drying step of rPET. PC/IPM PET flakes (rPET) (PolyQuest, https://www.polyquest.com/products/pet-and-rpet/) were used as received (not pre-dried). rPET flakes (12 g) and antioxidant (0.1 wt. %, Irganox 1010, Sigma) were fed into a hot conical twin screw compounder (DSM, Xplore, 15 cm³ capacity) equipped with co-rotating conical screws, recirculation channel allowing mixing during a controlled residence time and a circle die with diameter of 3.0 mm allowing for extrusion of the material from the compounder. The feeding/mixing/extrusion were performed under circulation of nitrogen, with a barrel temperature profile as follows: top position (270° C.), middle position (270° C.), and exit position (280° C.). The speed of rotation of the screws was 60 RPM. The extruder was filled in around 1.5 min. After feeding the extruder, the compound was mixed for 5 min. Then the sample was withdrawn directly through the die, extruded into an ice/water bath kept at 5° C.

The extrudate was cut into pieces of 3-5 mm length. Cut rPET pieces (2 g) were micronized using a centrifugal mill (Retsch ZM300) operating at 14000 RPM, cyclone-suction system for efficient air cooling and a ring sieve having an internal diameter of 10 cm and a mesh size of 0.5 mm. Feed rate was about 1 g/min, and the maximum ring sieve temperature (measured by attaching a type K thermocouple to the external wall of the ring sieve) was below 40° C. The milled sample was fractionated by sieving using an analytical sieve shaker (Retsch, AS200) operating at an amplitude of 0.7 mm for 10 minutes. Fractionation was performed using stainless steel test sieves (Retsch) with a diameter of 100 mm and mesh sizes of: 300 μm and 150 μm. The micronized PET fraction obtained between the 150 μm and 300 μm mesh sizes was used for enzymatic degradation tests.

### Comparative Example 10

Tightly Cross-Linked Network of rPET Using Araldite PT 910 (5 wt. %) as Reactive Agent.

This example presents a synthesis of a tightly cross-linked network of PC/IPM PET bottle waste by reactive mixing/extrusion and fast cooling followed by isothermal annealing at 280° C. for 20 min without pre-drying rPET. Commercial cross-linker Araldite PT 910 is used as reactive agent. Araldite PT 910 cross-linker is less expensive than DGT used in Example 34 and contains 85 mol % of DGT diepoxy (functionality f=4) and 15 mol % of triepoxy Tris(oxiranylmethyl) benzene-1,2,4-tricarboxylate (functionality f=6), as determined by 1H-NMR. The average functionality of Araldite PT 910 is f=4.3 (higher than that for DGT).

### Comparative Example 10.1: Reactive Mixing/Extrusion of rPET and Araldite PT 910 (5 wt. %) as Cross-Linker Followed by Fast Cooling

PC/IPM PET flakes (rPET) (PolyQuest, https://www.polyquest.com/products/pet-and-rpet/) were used as received (not pre-dried). rPET flakes (11.4 g), reactive agent Araldite PT 910 (Huntsman, 5 wt. %, 600 mg, 0.386 mmol epoxy/g rPET) and antioxidant (0.1 wt. %, Irganox 1010, Sigma) were fed into a hot conical twin screw compounder (DSM, Xplore, 15 cm³ capacity) equipped with co-rotating conical screws, recirculation channel allowing mixing during a controlled residence time and a circle die with diameter of 3.0 mm allowing for extrusion of the material from the compounder. The feeding/mixing/extrusion were performed under circulation of nitrogen, with a barrel temperature profile as follows: top position (270° C.), middle position (270° C.), and exit position (280° C.). The speed of rotation of the screws was 60 RPM. The extruder was filled in around 1.5 min. After feeding the extruder, the compound was mixed until the axial force reached 7000N. Then the sample was withdrawn directly through the die, extruded into an ice/water bath kept at 5° C.

A fraction of the extrudate material was cut into pieces of 3-5 mm length. Cut pieces (2 g) were micronized using a centrifugal mill (Retsch ZM300) operating at 14000 RPM, cyclone-suction system for efficient air cooling and a ring sieve having an internal diameter of 10 cm and a mesh size of 0.5 mm. Feed rate was about 1 g/min, and the maximum ring sieve temperature (measured by attaching a type K thermocouple to the external wall of the ring sieve) was below 40° C. The milled sample was fractionated by sieving using an analytical sieve shaker (Retsch, AS200) operating at an amplitude of 0.7 mm for 10 minutes. Fractionation was performed using stainless steel test sieves (Retsch) with a diameter of 100 mm and mesh sizes of: 300 μm and 150 μm. The micronized PET fraction obtained between the 150 μm and 300 μm mesh sizes was used for enzymatic degradation tests.

### Comparative Example 10.2: Tightly Cross-Linked rPET Network by Isothermal Annealing at 280° C. for 20 Min Using Araldite PT 910 as Cross-Linker

A fraction of the extrudate obtained in Comparative Example 10.1 was wiped with a paper tissue to remove water residue, was placed in a 1.5 mm thickness stainless steel mold and was annealed in an oven at 280° C. for 20 minutes. After isothermal annealing, the resulting material was fast cooled in a water bath at room temperature.

The obtained material was cut into pieces of around (3×3) mm. Cut pieces (2 g) were micronized using a centrifugal mill (Retsch ZM300) operating at 14000 RPM, cyclone-suction system for efficient air cooling and a ring sieve having an internal diameter of 10 cm and a mesh size of 0.5 mm. Feed rate was about 1 g/min, and the maximum ring sieve temperature (measured by attaching a type K thermocouple to the external wall of the ring sieve) was below 40° C. The milled sample was fractionated by sieving using an analytical sieve shaker (Retsch, AS200) operating at an amplitude of 0.7 mm for 10 minutes. Fractionation was performed using stainless steel test sieves (Retsch) with a diameter of 100 mm and mesh sizes of: 300 μm and 150 μm. The micronized PET fraction obtained between the 150 μm and 300 μm mesh sizes was used for enzymatic degradation tests.

In this Comparative Example, by using the cross-linker Araldite PT 910 with a higher functionality than DGT at the same wt. % concentration as pure DGT and for the same annealing time, the gel point of the system presented here (rPET+Araldite PT 910) is lower and forms a tighter network, respect to rPET+DGT presented in Example 34.

### Example 35

### Differential Scanning Calorimetry (DSC):

DSC first heating scans of materials were measured using a calorimeter (TA, discovery Q200). 10 mg of the extrudate material were cut and introduced in a capsule (TA, Tzero Pan T 220228 and Tzero Hermetic Lid T 220315). The following DSC procedure was performed for materials obtained in Example 34, Comparative Example 9, Comparative Example 10.1 and Comparative Example 10.2.
1) Equilibrate temperature from room temperature to 0° C.;
2) Isothermal step (0° C.) for 1 min;
3) Heating step from 0° C. to 290° C. at a heating rate of 10° C./min. 4) Equilibrate at 290° C. for 3 min;
5) Cooling step from 290° C. to 0° C. at a cooling rate of 20° C./min.
6) Equilibrate temperature at 0° C. for 1 min;
7) Heating step from 0° C. to 290° C. at a heating rate of 10° C./min.

### First Heating Scan:

Glass transition temperature (T_{g1}) taken as the midpoint of the transition.
Cold crystallization temperature (T_{c1}) taken as the peak temperature of the crystallization exothermic peak at around 100-140° C. Let us notice that some examples do not show cold crystallization.
Cold crystallization enthalpy (ΔH_{c1}) taken as the peak area of the crystallization exothermic peak at around 100-140° C. from visually determined respective starting points to end points using a straight baseline between them.
Melting peak temperature (Tₘ₁) taken as the peak temperature positioned at the highest temperature in the cases of multiple peaks in a range of 200-255° C.
Melting peak enthalpy (ΔHₘ₁) taken as the peak area of the endothermic melting peak at around 200-255° C. from visually determined respective starting points to end points using a straight baseline between them.

### Cooling Scan:

Crystallization from the melt temperature (T_{cfm}), taken as the peak temperature of the exotherm peak in a range of 110-230° C.
Crystallization from the melt enthalpy (ΔH_{cfm}), taken as the peak area of the exothermic crystallization peak at around 110-230° C. from visually determined respective starting points to end points using a straight baseline between them.

### Second Heating Scan:

Glass transition temperature second scan (T_{g2}) taken as the midpoint of the transition.
Cold crystallization temperature second scan (T_{c2}) taken as the peak temperature positioned at the highest temperature in the cases of multiple peaks in a range of 100-140° C. Let us notice that some examples do not show cold crystallization.
Cold crystallization enthalpy second scan (ΔH_{c2}) taken as the peak area of the crystallization exothermic peak at around 100-140° C. from visually determined respective starting points to end points using a straight baseline between them.
Melting peak temperature second scan (Tₘ₂) taken as the peak temperature of the endothermic melting peak at around 200-255° C.
Melting peak enthalpy second scan (ΔHₘ₂) taken as the peak area of the endothermic melting peak at around 200-255° C. from visually determined respective starting points to end points using a straight baseline between them.

Table 7 summarizes the thermal characterization by DSC of materials obtained in Example 34, Comparative Example 9, Comparative Example 10.1 and Comparative Example 10.2.

TABLE 7
DSC characterization of the sample obtained in Example 34, Comparative
Example 9, Comparative Example 10.1 and Comparative Example 10.2.

| | First heating scan | | | | | Cooling scan | | Second heating scan | | | | |
|---|---|---|---|---|---|---|---|---|---|---|---|---|
| | (10° C./min) | | | | | (20° C./min) | | (10° C./min) | | | | |
| Example | Tg1 | Tc1 | ΔHc1 | Tm1 | ΔHm1 | Tcfm | ΔHcfm | Tg2 | Tc2 | ΔHc2 | Tm2 | AHm2 |
| Example 34 | 68 | 136 | 23 | 220 | 28 | 143 | 5 | 77 | 146 | 21 | 225 | 28 |
| Comparative | 73 | 123 | 31 | 251 | 52 | 198 | 48 | 77 | — | — | 250 | 45 |
| Example 9 | | | | | | | | | | | | |
| Comparative | 71 | 124 | 28 | 240 | 38 | 174 | 42 | 73 | — | — | 233 | 39 |
| Example 10.1 | | | | | | | | | | | | |
| Comparative | 75 | 136 | 23 | 229 | 33 | 160 | 38 | 76 | — | — | 229 | 37 |
| Example 10.2 | | | | | | | | | | | | |

It is worth noting that by using 5 wt. % of cross-linker DGT (Example 34) or Araldite PT 910 (Comparative Example 10.2) the displacement of Tc1 respect to that of rPET (Comparative Example 9) is of around +13° C. However, the tightly cross-linked network of rPET+Araldite PT 910 (5 wt. %) (Comparative Example 10.2) has a much higher T_{g1} (75° C.) than that corresponding to rPET+DGT (5 wt. %) (68° C.), presented in Example 34.

### Example 36

### Enzymatic Depolymerization Yield at 10h of Reaction Using the LCC Variant at 75° C.

This example describes the procedure to estimate the reaction yield for the enzymatic depolymerization of polyester PC/IPMs at 10h of reaction by measuring the absorbance of plastic particle suspensions.

Depolymerization reaction yield measured by terephthalic acid (TPA) equivalent production and enzymatic depolymerization reaction time are referred below as “reaction yield” and “reaction time”, respectively.

### Calibration Curve for Determination of Terephthalic Acid (TPA) Equivalent in the Reaction Bath

Upon enzymatic depolymerization of PC/IPM or materials made of PC/IPM containing esters of terephthalic acid and diols, TPA and/or soluble low molecular weight molecules such as mono(2-hydroxyethyl) terephthalate and bis(2-hydroxyethyl) terephthalate for example, are released into the solution as depolymerization products. TPA has a maximal absorption band in UV-visible spectrum at 242 nm. UV-Visible spectra were recorded using Clariostar LVis plate from BMG Labtech. All other soluble molecules containing esters of terephthalic acid contribute to the absorbance signal as well. A calibration curve (Absorbance at 242 nm vs. TPA concentration) was obtained by measuring the absorbance of TPA (provided by Sigma Aldrich, purity 98%, used as received) aqueous solutions of NaOH 0.5 wt. % in milli-Q water of known concentrations. A linear fit of the calibration curve gives the following equation:

### Equation 4:

$$Absorbance at 242 nm ( a . u . ) = 70.47 ( L / g ) * [ TPA ] ( g / L ) \qquad Equation 4$$

In what follows we use this calibration curve to convert the absorbance signal into the TPA concentration as if only TPA was produced. This method is called determination of reaction yield by determination of TPA equivalent.

### Determination of TPA Equivalent Reaction Yield at a Reaction Time of 10 h.

From the calibration curve presented in Equation 4, it is possible to obtain the concentration of TPA as the enzymatic depolymerization proceeds, and calculate the corresponding reaction yield at a certain reaction time. For an enzymatic depolymerization assay, around 5 mg (Sartorius CP224S, precision 0.1 mg) of milled PC/IPM in the range between 150-300 μm was weighted in a 2 mL Eppendorf vial. 1 mL of potassium phosphate buffer 1M (pH 8), prepared from potassium phosphate monobasic (H₂KPO₄) and potassium phosphate dibasic (HK₂PO₄) (Sigma Aldrich), was added in the Eppendorf vial. The Eppendorf vial was then cooled to 0° C. in ice. Depolymerization assays were carried out using the LCC variant.

In each vial, a volume of 9.43*(m/5) μL of the LCC variant was added to reach a final concentration of 2 mg of enzyme per g of polyester in the vial, were m is the weighted mass in mg of plastic waste. Then, the Eppendorf vial was closed, sealed with PTFE film to prevent/minimize evaporation, and incubated in an Eppendorf Thermomixer at a 75° C. and shaken at 1200 rpm during 10 hours.

At 10 h of reaction, the PTFE film was removed and a 2 μL aliquot was taken from the reaction medium and was diluted (if required) by a factor of 5, 10 or 20 in NaOH 0.5 wt. % solution to ensure that the absorbance at 242 nm was in the linear range of TPA calibration curve presented in Eq. 4. The UV-Visible absorbance spectrum was recorded between 220 nm and 800 nm using a Clariostar LVis plate (BMG Labtech). The reaction yield was calculated as the concentration of TPA equivalents produced at 10h in reference to the maximum TPA concentration (g/L) corresponding to 100% reaction yield, as follows:

### Eq. 5:

$$Reaction yield ( \% ) = \frac{( \frac{{A}_{242 nm} {f}_{d}}{70.47} )}{\frac{m}{V} ( \frac{{M}_{A}}{{M}_{R U}} )} * 100 \qquad Equation ( 5 )$$

where A_{242 nm} is the absorbance of 2 μL (diluted) aliquot, f_{d} is the dilution factor of the aliquot, m is the weighted mass of plastic material waste of the assay (in g), V is the reaction volume (in L) M_{A} is the molecular weight of TPA, and M_{RU} is the molecular weight of the repeating unit of PET.

The reaction yield at 10 h was obtained by averaging the reaction yield of triplicate samples, i.e. 3 aliquots taken from 3 different vials in the same conditions, with error bars corresponding to the standard deviation of the three measurements.

Enzymatic depolymerization reaction yield at 10 h of milled and sieved samples described in Example 34, Comparative Example 9, Comparative Example 10.1, Comparative Example 10.2 and the material obtained in Example 8 milled and sieved under conditions detailed in Example 23 are presented in Table 8.

TABLE 8
enzymatic degradation yields after
10 h using the LCC variant at 75° C.

| | Reaction yield | |
|---|---|---|
| | [%] at 10 hs | STD |
| Comparative Example 9 | 83 | 3 |
| Comparative Example 10.1 | 63 | 7 |
| Comparative Example 10.2 | 79 | 6 |
| Example 8 (milled and | 95 | 3 |
| sieved as in Example 23) | | |
| Example 34 | 95 | 2 |

The previous results show that by using the same concentration (5 wt. %) and annealing time for Araldite PT 910 (Comparative Example 10.2) as for DGT (Example 34), pretreatment with Araldite PT 910 does not improve the depolymerization reaction yield as illustrated in the Table 8 above.

The results also show that there is no significant difference in enzymatic depolymerization yield at 10 hs between pretreated materials obtained by pre-drying rPET flakes (Example 8) and not pre-drying rPET flakes (Example 34).

### Example 36

### Particle Size Distribution

This example provides characterization of particle size distribution of the fractions of particles used for enzymatic degradation tests corresponding to pretreated PC/IPM described in Example 34, Comparative Example 9, Comparative Example 3 (milled and sieved under conditions detailed in Example 23) and Example 8 (milled and sieved under conditions detailed in Example 23).

Particle size distribution of the fractions of particles was measured by Laser Diffraction using a Microtrac Sync in the wet mode and Diffraction/Imaging Sync Analysis Type. 70 mg of micronized and sieved fraction were dispersed by mechanical stirring in 10 mL of distilled water. Dispersion of particles was loaded into the Microtrac Sync up to a loading factor of about 0.45. A sonication step of 20 seconds was performed prior to the measurement. Acquisition time was 30 seconds. A summary of results is presented in Table 9.

TABLE 9
Characterization of particle size distribution

| | | | | Percentiles, Volume | | |
|---|---|---|---|---|---|---|
| | M_{V} | M_{N} | M_{A} | distribution | | |
| | (μm) | (μm) | (μm) | 10% Tile | 50% Tile | 90% Tile |
| Example 8 (milled and | 304 | 170 | 282 | 223 | 311 | 376 |
| sieved as in Example 23) | | | | | | |
| Comparative Example 3 | 284 | 87 | 223 | 147 | 260 | 424 |
| (milled and sieved as | | | | | | |
| in Example 23) | | | | | | |
| Example 34 | 251 | 146 | 227 | 173 | 254 | 328 |
| Comparative | 264 | 97 | 215 | 144 | 256 | 382 |
| Example 9 | | | | | | |

where M_{V} is the mean diameter, in microns, of the volume distribution, M_{N} is the mean diameter, in microns, of the number distribution and M_{A} is the mean diameter, in microns, of the area distribution.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of processing a polymeric material comprising a crystallizable polymer or copolymer, comprising:
exposing a polymeric material to a polymer-degrading enzyme at a temperature of at least 20° C. for a duration of less than or equal to 4 days to obtain a reaction yield, wherein the reaction yield is at least 15%.

2. The method according to claim 1, wherein the polymer-degrading enzyme comprises a hydrolase, esterase, protease, cutinase, lipase, oxidase, peroxidase, and/or amidase.

3. (canceled)

4. The method according to claim 1, wherein the polymer-degrading enzyme is selected from Table 1.

5. The method according to claim 1, wherein exposing the polymeric material to the polymer-degrading enzyme occurs at a temperature equal to or higher than a glass transition temperature T_{g} of the crystallizable polymer or copolymer.

6. The method according to claim 1, wherein exposing the polymeric material to the polymer-degrading enzyme occurs at a temperature in a range from 15° C. lower than a glass transition temperature of the crystallizable polymer or copolymer to 120° C.

7. The method according to claim 6, wherein the temperature is in a range from 10° C. lower than the glass transition temperature of the crystallizable polymer or copolymer to 95° C.

8. (canceled)

9. The method according to claim 1, wherein exposing the polymeric material to the polymer-degrading enzyme occurs for a duration in a range from 10 minutes to 4 days.

10. The method according to claim 1, wherein exposing the polymeric material to the polymer-degrading enzyme for the duration results in a reaction yield in a range from 15% to 99%.

11. (canceled)

12. The method according to claim 1, further comprising reacting the polymeric material comprising the crystallizable polymer or copolymer with a reactive agent prior to exposure to the polymer-degrading enzyme.

13. The method according to claim 12, wherein the reactive agent comprises one or more epoxy, glycidyl, anhydride, glyceryl, boronic acid, boronate ester, maleimide, dioxaborolane, thioester, polysulfide, aldehyde, amine, acetoacetate ester, radical, furan, and/or olefin-containing groups.

14. (canceled)

15. (canceled)

16. The method according to claim 12, wherein the reactive agent comprises diglycidyl terephthalate (DGT), bisphenol A diglycidyl ether (DGEBA), novolac resin, cycloaliphatic epoxy, diglycidyl benzenedicarboxylate, triglycidyl benzene tricarboxylate, triglycidyl isocyanurate, epoxidized styrene-acrylic copolymer, diglycidyl phthalate, resorcinol diglycidyl ether, tetrabromobisphenol A diglycidyl ether, bisphenol F diglycidyl ether, 3,4-epoxycyclohexylmethyl-3′-4′-epoxycyclohexane carboxylate, tetraglycidyl methylene dianiline, triglycidyl glycerol, poly(glycolic acid), 1,4-butanediol diglycidyl ether, N,N′-bis[3(carbo-2′,3′-epoxypropoxy)phenyl]pyromellitimide, bis(3,4-epoxycyclohexylmethyl)adipate, 3,4-epoxycyclohexylmethyl-3,4-epoxycyclohexylate, 1,4-cyclohexanedimethanol diglycidyl ether, 4,4′-methylene-bisphenyl isocyanate, hexamethylene diisocyanate, 1,6-diisocyanato hexane, poly(phenyl isocyanate-co-formaldehyde), polymeric methylene diphenyl isocyanate, bisphenol-A dicyanate, pyromellitic dianhydride, trimellitic anhydride, a polyol, a polysulfide, a chain extender, and/or a maleimide-bearing diaxaborolane.

17. (canceled)

18. The method according to claim 1, wherein the crystallizable polymer or copolymer comprises a polyester, a polyamide, a polyolefin, a polystyrene, a fluoropolymer, a crystallizable thermoplastic polyurethane, a polyether ether ketone, a copolymer, and/or a combination thereof.

19. The method according to claim 1, wherein the crystallizable polymer or copolymer comprises polyethylene terephthalate.

20. (canceled)

21. The method according to claim 1, wherein the polymeric material comprising the crystallizable polymer or copolymer comprises a post-consumer and/or post-industrial polymeric material.

22. (canceled)

23. The method according to claim 12, wherein the reactive agent reacts with the polymeric material comprising the crystallizable polymer or copolymer in a transesterification, transcarbamoylation, transalkylation, transamination, siloxane-silanoate exchange, thiol-disulfide exchange, imine amine exchange, vinylogous urethane exchange, olefin metathesis, disulfide metathesis, dioxaborolane metathesis, nitroxide radical coupling, and/or Diels Alder cycloaddition reaction.

24. (canceled)

25. (canceled)

26. The method according to claim 12, wherein reacting the polymeric material comprising the crystallizable polymer or copolymer with the reactive agent comprises mixing a mixture comprising the polymeric material comprising the crystallizable polymer or copolymer and the reactive agent, extruding or mixing the mixture at a temperature above a melting temperature of the crystallizable polymer or copolymer, thermally annealing the mixture, fast cooling the mixture, and/or milling the mixture.

27. The method according to claim 12, wherein a concentration of the reactive agent in the mixture is in a range from 0.5 wt. % to 20 wt. %.

28.-33. (canceled)

34. A method of processing a polymeric material comprising a crystallizable polymer or copolymer, comprising:
exposing a polymeric material to a polymer-degrading enzyme selected from Table 1 to obtain a reaction yield, wherein the reaction yield is at least 15%.

35.-41. (canceled)

42. The method according to claim 34, wherein the polymeric material comprises a plurality of particles having an average particle size in a range from 0.3 mm to 2 mm.

43. A system configured to implement the method of claim 34.
